(19) United States
(12) Patent Application Publication
(10) Pub. No.: US 2024/0366684 A1
(43) Pub. Date: Nov. 7, 2024

(54) COMPOSITIONS COMPRISING A ZIM3 EFFECTOR AND METHODS OF USE THEREOF

(71) Applicant: The George Washington University, Washington, DC (US)

(72) Inventors: Emilia Entcheva, Washington, DE (US); Julie Leann Han, Yardley, PA (US)

(21) Appl. No.: 18/653,895

(22) Filed: May 2, 2024

Related U.S. Application Data
(60) Provisional application No. 63/463,515, filed on May 2, 2023.

(51) Int. Cl. A61K 35/545; A61P 9/06; C12N 5/074; C12N 9/22; C12N 15/11; G01N 33/50

(52) U.S. Cl. CPC A61K 35/545; A61P 9/06; C12N 5/0696; C12N 9/22; C12N 15/111; G01N 33/5014; G01N 33/5073; C12N 2310/20

(57) ABSTRACT

Disclosed herein are nucleic acid molecules, viral vectors, plasmids, and pharmaceutical formulations for use as effectors in methods of maturing iPSC-CMs. Disclosed herein are methods of maturing iPSC-CMs.

### CROSS-REFERENCE TO RELATED APPLICATIONS

This application claims the benefit of priority to U.S. Provisional Patent Application No. 63/463,515 filed 2 May 2023, which is incorporated by reference herein in its entirety.

### REFERENCE TO THE SEQUENCE LISTING

The Sequence Listing submitted 2 May 2024 as an XML file named “GW127 (091019-795298)-Sequence Listing”, created on 2 May 2024 and having a size of 245 kilobytes is hereby incorporated by reference pursuant to 37 C.F.R. § 1.52(e)(5).

### BACKGROUND

Cardiovascular diseases are a leading cause of morbidity and mortality in developed countries. They usually result in cardiomyocyte death. Although there are indications that human adult heart has certain level of endogenous regeneration capacity, with different estimations of the rate of cardiomyocyte turnover between studies, adult human heart cannot effectively regenerate after injury. Therefore, loss of cardiomyocytes causes permanent damage of heart that progressively decreases its functionality and could eventually lead to heart failure and death.

Current treatments of cardiac disorders are mostly based on symptomatic treatment by medications and implantable cardiac devices. While heart transplantation constitutes the ultimate treatment for severe stages of heart failure, there are serious difficulties connected with organ transplantation such as limitations in organ supply and immunological incompatibility. Therefore, providing new tools for treatment of cardiovascular diseases, such as cardiac ischemia, myocardial infarction, and heart failure, is obviously needed. Theoretically, de novo cardiomyocytes for cell replacement therapy could potentially solve the problem of availability of human cardiac tissue.

While induced pluripotent stem cell cardiac myocytes (iPSC-CMS) can couple efficiently to the damaged heart and restore cardiac contractility, almost all found iPSC-CM transplantation is arrhythmogenic, thus hampering the use of iPSC-CMs for cardiac regeneration. Studies show that iPSC-CM cultures are highly heterogeneous containing atrial-, ventricular- and nodal-like CMs. Furthermore, they have an immature phenotype, resembling more fetal than adult CMs.

There is an urgent need to overcome these issues. These issues are solved by the disclosed compositions and methods.

### BRIEF DESCRIPTION OF THE FIGURES

FIG. 1 shows a summary of maturation/anti-arrhythmic effects of dCas9/Zim3 expressing human iPSC-CMs when compared to unmodified control.

FIG. 2 shows the endogenous gene expression of Zim3/ZNF657/ZNF264 in human adult heart (left ventricle, LV) from GTEx and in human iPSC-CMs along a well-expressed cardiac gene (KCNH2). The Zim3 and ZNF657 gene identifiers were either not expressed or expressed in only a small subset of samples at negligible levels. For ZNF264 and for KCNH2, the log 10 of normalized TPM-log 10(TPM/GM)—is shown.

FIG. 3A-FIG. 3G show the effects of dCas9/Zim3 expression in human iPSC-CMs: RNA, protein, and electrophysiological consequences. FIG. 3A shows the experimental workflow with all-optical electrophysiology (Heinson Y W, et al. (2023) J Biomed Opt. 28 (1): 016001) and molecular characterization. FIG. 3B shows the expression of mCherry tagged constructs (mCherry, MeCP2-KRAB-dCas9-mCherry, Zim3-KRAB-dCas9-mCherry) in human iPSC-CMs. Scale bar=10 μm. FIG. 3C shows the functional results from all-optical electrophysiology: changes in APD and CTD under 0.5 Hz pacing (n=9-34 biologically independent samples). FIG. 3D shows the relative changes in spontaneous frequency (delta) with respect to the mean for the dCas9/Zim3 group for each run (n=14-40 biologically independent samples per group). FIG. 3E shows the changes in conduction shown in example activation maps and as normalized conduction velocity (CVₙₒᵣₘ) at 0.75 Hz and 1.0 Hz pacing (n=2-9 biologically independent samples). Data shown as mean±S.E.M. FIG. 3F shows the molecular analysis (qPCR) in pristine samples (top) and after all-optical electrophysiology (bottom)—KCNH2, GJA1, and KCHJ2 (n=4-16 biologically independent samples, n=3 technical replicates). FIG. 3G shows a Western blot (Wes by ProteinSimple) of Cx43 post all-optical electrophysiology (n=6-13 biologically independent samples). Statistical comparisons were done using one-way ANOVA with post-hoc Tukey correction. In all cases **p<0.01. Biorender was used.

FIG. 4 shows that qPCR did not detect the very low endogenous levels in the (non-infected) human iPSC-CMs, but properly captured the overexpression of dCas9-KRAB-Zim3 at MOI 1000. Several independent runs with sufficient “n” (n=11-12 biological samples per group) were used to validate the qPCR.

FIG. 5 shows optically paced action potentials (red—also indicated with arrow heads) and calcium transients (green) at 0.75 Hz pacing show the shortening of APD (and CTD to some degree) for dCas9-Zim3 compared to control and mCherry virally transduced samples. Note that dCas9-MeCP2 also shortened APD but to a lesser degree than Zim3.

FIG. 6A shows the percent change of APD and CTD and 0.5 Hz (n=1-10 biologically independent samples; two-way ANOVA) and 0.75 Hz pacing (n=2-10 biologically independent samples; two-way ANOVA). Data was normalized to Scramble/Zim3 samples treated with DMSO control. FIG. 6B shows the percent change in APD triangulation at 0.5 Hz (n=4-10 biologically independent samples; two-way ANOVA) and 0.75 Hz (n=3-10 biologically independent samples; two-way ANOVA) pacing. Data was normalized to Scramble/Zim3 samples treated with DMSO control. FIG. 6C displays the percentage of paced samples with alternans and blocks. (*) indicates significance of p≤0.05. (**) indicates significance of p≤0.05. Data are presented as mean±SEM.

FIG. 7 shows the plasmid pHR-UCOE-SFFV-dCas9-mCherry-Zim3-KRAB.

FIG. 8 shows the MeCP2-KRAB-dCas9-mCherry adenoviral vector (pAV[Exp]-mCherry-SFFV-dCas9/KRAB/MeCP2), which was used at MOI 1000.

FIG. 9 shows the plasmid 154473-attachment_w5DDpndhwpbDmnEJw54.

### BRIEF SUMMARY

Disclosed herein is a nucleic acid molecule, comprising: a nucleic acid sequence encoding (i) at least one zinc finger protein, (ii) at least one polypeptide having effector activity, and (iii) a deactivated Cas9 (dCas9) endonuclease. Disclosed herein is a nucleic acid molecule, comprising: a sequence encoding a Zim3-KRAB-dCas9 effector.

Disclosed herein is a viral vector or a non-viral vector comprising a nucleic acid sequence encoding (i) at least one zinc finger protein, (ii) at least one polypeptide having effector activity, and (iii) a deactivated Cas9 (dCas9) endonuclease. Disclosed herein is a viral vector or a non-viral vector comprising a nucleic acid sequence encoding a Zim3-KRAB-dCas9 effector.

Disclosed herein is a plasmid comprising a nucleic acid sequence encoding (i) at least one zinc finger protein, (ii) at least one polypeptide having effector activity, and (iii) a deactivated Cas9 (dCas9) endonuclease. Disclosed herein is a plasmid comprising a nucleic acid sequence encoding a Zim3-KRAB-dCas9 effector.

Disclosed herein is an iPSC-CM transduced by a nucleic acid molecule comprising a nucleic acid sequence encoding (i) at least one zinc finger protein, (ii) at least one polypeptide having effector activity, and (iii) a deactivated Cas9 (dCas9) endonuclease.

Disclosed herein is an iPSC-CM transduced by a nucleic acid molecule comprising a sequence encoding a Zim3-KRAB-dCas9 effector.

Disclosed herein is an iPSC-CM transfected by a viral vector comprising a nucleic acid sequence encoding (i) at least one zinc finger protein, (ii) at least one polypeptide having effector activity, and (iii) a deactivated Cas9 (dCas9) endonuclease.

Disclosed herein is an iPSC-CM transfected by a viral vector comprising a nucleic acid sequence encoding a Zim3-KRAB-dCas9 effector.

Disclosed herein is an iPSC-CM transduced by a non-viral vector comprising a nucleic acid sequence encoding (i) at least one zinc finger protein, (ii) at least one polypeptide having effector activity, and (iii) a deactivated Cas9 (dCas9) endonuclease.

Disclosed herein is an iPSC-CM transduced by a non-viral vector comprising a nucleic acid sequence encoding a Zim3-KRAB-dCas9 effector.

Disclosed herein is a pharmaceutical formulation comprising a nucleic acid molecule comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier.

Disclosed herein is a pharmaceutical formulation comprising a nucleic acid molecule comprising a nucleic acid sequence encoding a Zim3-KRAB-dCas9 effector and a pharmaceutically acceptable carrier.

Disclosed herein is a pharmaceutical formulation comprising a disclosed viral vector comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier.

Disclosed herein is a pharmaceutical formulation comprising a disclosed viral vector comprising a nucleic acid sequence encoding a Zim3-KRAB-dCas9 effector and a pharmaceutically acceptable carrier.

Disclosed herein is a pharmaceutical formulation comprising a disclosed non-viral vector comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier.

Disclosed herein is a pharmaceutical formulation comprising a disclosed non-viral vector comprising a nucleic acid sequence encoding a Zim3-KRAB-dCas9 effector and a pharmaceutically acceptable carrier.

Disclosed herein is a pharmaceutical formulation comprising a disclosed recombinant viral vector comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier.

Disclosed herein is a pharmaceutical formulation comprising a disclosed recombinant non-viral vector comprising a disclosed nucleic acid molecule and a pharmaceutically acceptable carrier. Disclosed herein is a pharmaceutical formulation comprising a disclosed recombinant non-viral vector comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier.

Disclosed herein is a pharmaceutical formulation comprising iPSC-CMs transduced by a nucleic acid molecule comprising a nucleic acid sequence encoding (i) at least one zinc finger protein, (ii) at least one polypeptide having effector activity, and (iii) a deactivated Cas9 (dCas9) endonuclease.

Disclosed herein is a pharmaceutical formulation comprising iPSC-CMs transduced by a nucleic acid molecule comprising a sequence encoding a Zim3-KRAB-dCas9 effector.

Disclosed herein is a pharmaceutical formulation comprising iPSC-CMs transfected by a viral vector comprising a nucleic acid sequence encoding (i) at least one zinc finger protein, (ii) at least one polypeptide having effector activity, and (iii) a deactivated Cas9 (dCas9) endonuclease.

Disclosed herein is a pharmaceutical formulation comprising iPSC-CMs transfected by a viral vector comprising a nucleic acid sequence encoding a Zim3-KRAB-dCas9 effector.

Disclosed herein is an implantable cardiac patch comprising one or more iPSC-CMs transduced by a nucleic acid molecule comprising a nucleic acid sequence encoding (i) at least one zinc finger protein, (ii) at least one polypeptide having effector activity, and (iii) a deactivated Cas9 (dCas9) endonuclease.

Disclosed herein is an implantable cardiac patch comprising one or more iPSC-CMs transduced by a nucleic acid molecule comprising a sequence encoding a Zim3-KRAB-dCas9 effector.

Disclosed herein is an implantable cardiac patch comprising one or more iPSC-CMs transfected by a viral vector comprising a nucleic acid sequence encoding (i) at least one zinc finger protein, (ii) at least one polypeptide having effector activity, and (iii) a deactivated Cas9 (dCas9) endonuclease.

Disclosed herein is an implantable cardiac patch comprising one or more iPSC-CMs transfected by a viral vector comprising a nucleic acid sequence encoding a Zim3-KRAB-dCas9 effector.

Disclosed herein is a method of improving or enhancing maturation of iPSC-CMs, the method comprising contacting one or more iPSC-CMs with a therapeutically effective amount of a disclosed nucleic acid molecule, wherein following the contacting step, the one or more iPSC-CMs are characterized by a mature structural, electrophysiological, contractile, and metabolic profile.

Disclosed herein is a method of improving or enhancing maturation of iPSC-CMs, the method comprising contacting one or more iPSC-CMs with a therapeutically effective amount of a disclosed viral or non-viral vector, wherein following the contacting step, the one or more iPSC-CMs are characterized by a mature structural, electrophysiological, contractile, and metabolic profile.

Disclosed herein is a method of slowing and/or preventing progression of a cardiac disease or disorder in a subject, the method comprising administering to a subject a therapeutically effective amount of a disclosed nucleic acid molecule, thereby reducing the pathological phenotype associated with the cardiac disease or disorder.

Disclosed herein is a method of slowing and/or preventing progression of a cardiac disease or disorder in a subject, the method comprising administering to a subject a therapeutically effective amount of a disclosed viral vector or non-viral vector, thereby reducing the pathological phenotype associated with the cardiac disease or disorder.

Disclosed herein is a method of slowing and/or preventing progression of a cardiac disease or disorder in a subject, the method comprising administering to a subject a therapeutically effective amount of a disclosed pharmaceutical formulation, thereby reducing the pathological phenotype associated with the cardiac disease or disorder.

Disclosed herein is a method of slowing and/or preventing progression of a cardiac disease or disorder in a subject, the method comprising implanting in a subject a disclosed cardiac patch, thereby reducing the pathological phenotype associated with the cardiac disease or disorder.

Disclosed herein is a method of slowing and/or preventing progression of a cardiac disease or disorder in a subject, the method comprising administering to a subject a therapeutically effective amount of one or more disclosed iPSC-CMs, thereby reducing the pathological phenotype associated with the cardiac disease or disorder.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Relevant Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

In an aspect, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

In an aspect, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

In an aspect, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a cardiac disease or a cardiac disorder, can be suspected of having a cardiac disease or a cardiac disorder, or can be at risk of developing and/or acquiring a cardiac disease or a cardiac disorder.

In an aspect, the term “diagnosed” means having been subjected to an examination by a person of skill, for example, a physician, and found to have a condition that can be diagnosed or treated by one or more of the disclosed agents, disclosed therapeutic agents, disclosed pharmaceutical formulations, disclosed nucleic acid molecules, disclosed vectors, disclosed cardiac patches, disclosed plasmids, disclosed iPSC-CMs, or any combination thereof, or by one or more of the disclosed methods. For example, “diagnosed with a cardiac disease or a cardiac disorder” means having been subjected to an examination by a person of skill, for example, a physician, and found to have a condition that can be treated by one or more of the disclosed isolated nucleic acid molecules, disclosed viral vectors, disclosed vectors, disclosed pharmaceutical formulations, disclosed iPSC-CMs, disclosed plasmids, or any combination thereof, or by one or more of the disclosed methods. For example, “suspected of having a cardiac disease or a cardiac disorder” can mean having been subjected to an examination by a person of skill, for example, a physician, and found to have a condition that can likely be treated by one or more of the disclosed isolated nucleic acid molecules, disclosed viral vectors, disclosed non-viral vectors, disclosed pharmaceutical formulations, disclosed iPSC-CMs, disclosed plasmids, or any combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” can refer to a subject that has been diagnosed with or is suspected of having a cardiac disease or a cardiac disorder. In an aspect, a patient can refer to a subject that has been diagnosed with or is suspected of having a cardiac disease or a cardiac disorder and is seeking treatment or receiving treatment for a cardiac disease or a cardiac disorder.

In an aspect, the phrase “identified to be in need of treatment for a disorder,” or the like, refers to selection of a subject based upon need for treatment of the disorder. For example, a subject can be identified as having a need for treatment of a disorder (e.g., such as a cardiac disease or a cardiac disorder) based upon an earlier diagnosis by a person of skill and thereafter subjected to treatment for the disorder (e.g., a cardiac disease or a cardiac disorder). In an aspect, the identification can be performed by a person different from the person making the diagnosis. In an aspect, the administration can be performed by one who performed the diagnosis.

In an aspect, “inhibit,” “inhibiting”, and “inhibition” mean to diminish or decrease an activity, level, response, expression, condition, severity, disease, or other biological parameter. This can include, but is not limited to, the complete ablation of the activity, level, response, expression, condition, severity, disease, or other biological parameter. This can also include, for example, a 10% inhibition or reduction in the activity, level, response, condition, severity, disease, or other biological parameter as compared to the native or control level (e.g., a subject not having a cardiac disease or a cardiac disorder). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-cardiac disease or a pre-cardiac disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as a cardiac disease or a cardiac disorder). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a cardiac disease or a cardiac disorder can reduce the severity of an established disease in a subject by 1%-100% as compared to a control (such as, for example, an individual not having a cardiac disease or a cardiac disorder). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a cardiac disease or a cardiac disorder. For example, treating a cardiac disease or a cardiac disorder can reduce one or more symptoms in a subject by 1%-100% as compared to a control (such as, for example, an individual not having a cardiac disease or a cardiac disorder). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a cardiac disease or a cardiac disorder. It is understood that treatment does not necessarily refer to a cure or complete ablation or eradication of a cardiac disease or a cardiac disorder. However, in an aspect, treatment can refer to a cure or complete ablation or eradication of a cardiac disease or a cardiac disorder. In an aspect, a native or control level can be a pre-disease or pre-cardiac disease or a pre-cardiac disorder level.

In an aspect, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

In an aspect, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

In an aspect, a “regulatory element” can refer to promoters, enhancers, internal ribosomal entry sites (IRES), and other expression control elements (e.g., transcription termination signals, such as polyadenylation signals and poly-U sequences). Regulatory elements are discussed infra and can include those that direct constitutive expression of a nucleotide sequence in many types of host cells and those that direct expression of the nucleotide sequence only in certain host cells (e.g., tissue-specific regulatory sequences).

In an aspect, “peptide,” “polypeptide,” and “protein” are used interchangeably, and refer to a compound comprised of amino acid residues covalently linked by peptide bonds. A protein must contain at least two amino acids and there is no limitation on the maximum number of amino acids that can comprise a protein's sequence. The term “peptide” can refer to a short chain of amino acids including, for example, natural peptides, recombinant peptides, synthetic peptides, or any combination thereof. Proteins and peptides can include, for example, biologically active fragments, substantially homologous polypeptides, oligopeptides, homodimers, heterodimers, variants of polypeptides, modified polypeptides, derivatives, analogs, and fusion proteins, among others.

“Nucleic acid” or “oligonucleotide” or “polynucleotide” In an aspect means at least two nucleotides covalently linked together. The depiction of a single strand can also define the sequence of the complementary strand. Thus, a nucleic acid can encompass the complementary strand of a depicted single strand. Many variants of a nucleic acid can be used for the same purpose as a given nucleic acid. Thus, a nucleic acid can encompass substantially identical nucleic acids and complements thereof. A single strand can provide a probe that can hybridize to a target sequence under stringent hybridization conditions. Thus, a nucleic acid can encompass a probe that hybridizes under stringent hybridization conditions. A nucleic acid can be single-stranded, or double-stranded, or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. Also, in an aspect, the terms “nucleic acid,” “nucleic acid molecule,” “nucleic acid construct,” “nucleotide sequence”, and “polynucleotide” can refer to RNA or DNA that is linear or branched, single or double stranded, or a hybrid thereof. The term can encompass RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. A “synthetic” nucleic acid or polynucleotide, in an aspect, refers to a nucleic acid or polynucleotide that is not found in nature but is constructed by the hand of man and therefore is not a product of nature.

A “polynucleotide” is a sequence of nucleotide bases, and may be RNA, DNA, or DNA-RNA hybrid sequences (including both naturally occurring and non-naturally occurring nucleotides).

A “fragment” or “portion” of a nucleotide sequence can be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of, or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the disclosure can be, where appropriate, included in a larger polynucleotide of which it is a constituent. In an aspect, a fragment or portion of a nucleotide sequence or nucleic acid sequence can comprise the sequence encoding an exon having one or more mutations. In an aspect, a fragment or portion of a nucleotide sequence or nucleic acid sequence can comprise a target of interest.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

A “heterologous” or a “recombinant” nucleotide or amino acid sequence as used interchangeably herein can refer to a nucleotide or an amino acid sequence not naturally associated with a host cell into which it is introduced, including non-naturally occurring multiple copies of a naturally occurring nucleotide or amino acid sequence.

Different nucleic acids or proteins having homology can be referred to as “homologues”. The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the disclosed compositions and disclosed methods can comprise homologues to the disclosed nucleotide sequences and/or disclosed polypeptide sequences.

“Orthologous,” in an aspect, can refer to homologous nucleotide sequences and/or amino acid sequences in different species that arose from a common ancestral gene during speciation. A homologue of a disclosed nucleotide sequence or a disclosed polypeptide can have substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to a disclosed nucleotide sequence or a disclosed polypeptide.

In an aspect, “complement” or “complementary” means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

In an aspect, extrusion based bioprinting can be used in modern day bioprinters, and most commercially available bioprinters are extrusion-based systems. The process for bioprinting is simple and in close resemblance to the operations of an inexpensive inkjet printer; however, the major difference is that inkjet printers deposit materials in a droplet fashion, while bioprinters deposit materials as strands. Extrusion based bioprinting is based on isolated cells that are mixed with a bioink and loaded onto a syringe, and then, pneumatic pressure is used to move the cell loaded bioink through the syringe tip. In addition to extrusion based bioprinting, there are additional modalities that include inkjet and laser induced forward transfer, which may be necessary for bioprinting at higher resolutions. The main advantage of inkjet bioprinting and laser induced forward transfer bioprinting is the high precision, higher than that obtained with extrusion based bioprinting. In the case of whole-heart bioprinting, extrusion based bioprinting will likely need to be coupled with higher resolution techniques for the placement of smaller structures, like the microvasculature and the nerves. While the selection of biomaterials used for tissue engineering is large, only a subset of these materials is suitable for applications in bioprinting. Soft hydrogels commonly used in bioprinting are fibrin, collagen, alginate, pluronic acid, agarose, and gelatin. Similar to other tissue engineering applications, the viability, purity, and concentration of the initial cell suspension being used are important. Important printing parameters include viscosity of the cell laden bioink, pneumatic pressure, printing speed, and tip diameter. High viscosity bioinks and smaller tip diameters require a higher printing pressure. The printing speed affects the diameter of the fibers, with higher speeds correlated with thinner fibers. Every bioprinting application is different and requires rigorous optimization of the bioprinting variables. Acute fine-tuning of printing parameters is required for any bioprinting application and varies significantly between tissue and organ printing applications.

In an aspect, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

Ubiquitous/constitutive promoters” are known to the art and include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

In an aspect, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

In an aspect, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (Sec, for example, genomes.urv.es/OPTIMIZER/).

In an aspect, “expression cassette” or “transgene cassette” can refer to a distinct component of vector DNA comprising a transgene and one or more regulatory sequences to be expressed by a transfected cell. Generally, an expression cassette or transgene cassette can comprise a promoter sequence, an open reading frame (i.e., the transgene), and a 3′ untranslated region (e.g., in eukaryotes a polyadenylation site).

In an aspect, the terms “human embryonic stem cell”, “hES cell”, and “hESC” can refer to cells derived, obtainable or originating from human embryos or blastocysts, which are self-renewing and pluri- or toti-potent, having the ability to yield all ell types present in a mature animal. Human embryonic stem cells (hESCs) can be isolated, for example, from human blastocysts obtained from human in vivo preimplantation embryos, in vitro fertilized embryos, or one-cell human embryos expanded to the blastocyst stage.

A “pluripotent stem cell” is one or more tissues or organs, or preferably three germ layers: endoderm (medial gastric mucosa, gastrointestinal tract, lung), mesodermal (muscle, bone, blood, genitourinary tract). Alternatively, it is a stem cell capable of differentiating into all the cells constituting any of the ectoderm (epidermal tissue and nervous system).

The terms “induced pluripotent stem cell and “iPSC” refer to cells derivable, obtainable or originating from human adult somatic cells of any type reprogrammed to a pluripotent state through the expression of exogenous genes, such as transcription factors, including OCT4, SOX1, SOX2, SOX3, SOX15, and SOX18, KLF4, LIN28, Glis 1 and c-MYC, although without limitation thereto.

In an aspect, the terms “differentiate”, “differentiating” and “differentiated”, relate to progression or maturation of a cell from an earlier or initial stage of a developmental pathway to a later or more mature stage of the developmental pathway. In an aspect, “differentiated” docs not mean or imply that the cell is fully differentiated and has lost pluropotentiality or capacity to further progress along the developmental pathway or along other developmental pathways. Differentiation may be accompanied by cell division. “Dedifferentiation” is a cellular process in which partially or finally differentiated cells return to an early developmental stage, eg, pluripotent or pluripotent. “Transdifferentiation” is the process of converting one differentiated cell type into another differentiated cell type. Typically, transdifferentiation by programming occurs without the cell undergoing an intermediate pluripotent phase-ie, the cell is programmed directly from one differentiated cell type to another. Under certain conditions, the proportion of offspring with new cell type characteristics may be at least about 1%, 5%, 25%, or higher, in more preferred order.

In an aspect, “cardiomyocytes” can refer to cardiac muscle cells also known as myocardiocytes or cardiac myocytes, that make up cardiac muscle such as found in the atria and ventricles of the heart. Each myocardial cell contains myofibrils, which are the fundamental contractile units of cardiac muscle cells. Cardiomyocytes typically contain one or two nuclei, although they may have as many as four and a relatively high mitochondrial density, facilitating production of adenosine triphosphate (ATP) for muscle contraction. Myocardial infarction causes the death of cardiomyocytes. In adults, the heart's limited capacity to regenerate these lost cardiomyocytes leads to compromised cardiac function and high morbidity and mortality.

In an aspect, the term “somatic cell” refers to any cell other than germ cells, such as eggs or sperm, and does not directly transfer its DNA to the next generation. Typically, somatic cells have limited or no pluripotency. The somatic cells used herein may be naturally occurring or genetically modified.

“Programming” is the process of changing the types of offspring a cell can produce. For example, if the cell is modified to produce at least one new cell type progeny either in culture or in vivo compared to a cell type that the cell should be able to produce under the same conditions without programming. The cell has been programmed. This is because a measurable proportion of such progeny is observed after sufficient proliferation, even though it was essentially unable to produce progeny with the phenotypic properties of the new cell type prior to programming; or new. It means that the proportion of cell type features is much higher than before programming. This process includes differentiation, dedifferentiation, and transdifferentiation.

In an aspect, “reprogramming” can confer on cells, either in culture or in vivo, the ability to form progeny of at least one new cell type that is measurable higher than the ability they would have under the same conditions without reprogramming. More specifically, reprogramming can be the process of imparting pluripotency to somatic cells. This is because if, after sufficient proliferation and before reprogramming, such progeny could not be essentially formed, then a measurable proportion of progeny with the phenotypic properties of the new cell type, otherwise new cells. It means that the proportion with type characteristics is measurable higher than before reprogramming. Under certain conditions, the proportion of offspring with new cell type characteristics can be at least about 1%, 5%, 25% or higher, in the preferred order.

In an aspect, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing progression of a cardiac disease or a cardiac disorder is intended. The words “prevent” and “preventing” and “prevention” also refer to prophylactic or preventative measures for protecting or precluding a subject (e.g., an individual) not having a cardiac disease or a cardiac disorder or a cardiac disease-related or a cardiac disorder-related complication from progressing to that complication.

In an aspect, the terms “administering” and “administration” refer to any method of providing one or more of the disclosed isolated nucleic acid molecules, disclosed pharmaceutical formulations, disclosed vectors, or any combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed therapeutic agent, a disclosed pharmaceutical composition, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent. In an aspect, administration can comprise grafting onto or implanting onto one or more portions of damaged or diseased portions of a subject's heart.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). In an aspect, the recitation of a biologically active agent inherently encompasses the pharmaceutically acceptable salts thereof.

In an aspect, a “therapeutic agent” can be any agent that effects a desired clinical outcome in a subject having a cardiac disease or a cardiac disorder, suspected of having a cardiac disease or a cardiac disorder, and/or likely to develop or acquire a cardiac disease or a cardiac disorder. In an aspect, a disclosed therapeutic agent can be an oligonucleotide therapeutic agent. A disclosed oligonucleotide therapeutic agent can comprise a single-stranded or double-stranded DNA, RNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

By “determining the amount” is meant both an absolute quantification of a particular analyte (e.g., an mRNA sequence) or a determination of the relative abundance of a particular analyte (e.g., an amount as compared to a mRNA sequence). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

In an aspect, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, by changing the duration of time one or more of the disclosed isolated nucleic acid molecules, disclosed vectors, disclosed pharmaceutical formulations, or a combination are administered to a subject, or by substituting for one or more of the disclosed components and/or reagents with a similar or equivalent component and/or reagent. The same applies to all disclosed therapeutic agents, immune modulators, immunosuppressive agents, proteosome inhibitors, etc.

In an aspect, a therapeutic agent can be a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. Examples include but are not limited to a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipinc, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and mocxipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolaminc hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopaminc, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antincoplastic agents such as etoposide, etoposide phosphate, cyclophosphamide, methotrexate, 5-fluorouracil, vincristine, doxorubicin, cisplatin, hydroxyurca, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). In an aspect, the recitation of a pharmaceutically active agent inherently encompasses the pharmaceutically acceptable salts thereof.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. Sequences with substantial sequence identity do not necessarily have the same length and may differ in length. For example, sequences that have the same nucleotide sequence but of which one has additional nucleotides on the 3′- and/or 5′-side are 100% identical.

In an aspect, the skilled person can determine an efficacious dose, an efficacious schedule, and an efficacious route of administration for one or more of the disclosed isolated nucleic acid molecules, disclosed pharmaceutical formulations, disclosed vectors, or any combination thereof so as to treat or prevent a cardiac disease or a cardiac disorder. In an aspect, the skilled person can also alter, change, or modify an aspect of an administering step to improve efficacy of one or more of the disclosed isolated nucleic acid molecules, disclosed pharmaceutical formulations, disclosed vectors, or any combination thereof. In an aspect, the skilled person can determine an efficacious dose, an efficacious schedule, and an efficacious route of administration for any disclosed isolated nucleic acid molecule, disclosed pharmaceutical formulation, disclosed vector, disclosed therapeutic agent, or any combination thereof.

In an aspect, “modifying the method” can comprise modifying or changing one or more features or aspects of one or more steps of a disclosed method. For example, in an aspect, a method can be altered by changing the amount of one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof administered to a subject, or by changing the frequency of administration of one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof, or by changing the duration of time that the one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof are administered to a subject.

In an aspect, “isolated” refers to a nucleic acid molecule or a nucleic acid sequence that has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated proteins or nucleic acids, or cells containing such, in some examples are at least 50% pure, such as at least 75%, at least 80%, at least 90%, at least 95%, at least 98%, or at least 100% pure.

In an aspect, “concurrently” means (1) simultaneously in time, or (2) at different times during the course of a common treatment schedule.

In an aspect, the term “contacting” refers to bringing one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof together with a target area or intended target area (e.g., diseased, infarcted, or ischemic cardiac tissue) in such a manner that the one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof can exert an effect on the intended target or targeted area (e.g., diseased, infarcted, or ischemic cardiac tissue) either directly or indirectly. A target area or intended target area can be one or more of a subject's organs (e.g., lungs, heart, liver, kidney, brain, etc.). In an aspect, a target area or intended target area can be any cell or any organ affected by a cardiac disease or a cardiac disorder. In an aspect, a target area or intended target area can be diseased, infarcted, or ischemic cardiac tissue.

In an aspect, “determining” can refer to measuring or ascertaining the presence and severity of a cardiac disease or a cardiac disorder. Methods and techniques used to determine the presence and/or severity of a cardiac disease or a cardiac disorder are typically known to the medical arts. For example, the art is familiar with the ways to identify and/or diagnose the presence, severity, or both of a cardiac disease or a cardiac disorder. In an aspect, “determining” can also refer to measuring or ascertaining the level of one or more proteins or peptides in a biosample, or measuring or ascertaining the level or one or more RNAs or miRNAs in a biosample. Methods and techniques for determining the expression and/or activity level of relevant proteins, peptides, mRNA, DNA, or any combination thereof known to the art and are disclosed herein.

In an aspect, “effective amount” and “amount effective” can refer to an amount that is sufficient to achieve the desired result such as, for example, the treatment and/or prevention of a cardiac disease or a cardiac disorder. In an aspect, the terms “effective amount” and “amount effective” can refer to an amount that is sufficient to achieve the desired an effect on an undesired condition (e.g., a cardiac disease or a cardiac disorder). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof that (i) treats the particular disease, condition, or disorder (e.g., a cardiac disease or a cardiac disorder), (ii) attenuates, ameliorates, or eliminates one or more symptoms of the particular disease, condition, or disorder (e.g., a cardiac disease or a cardiac disorder), or (iii) delays the onset of one or more symptoms of the particular disease, condition, or disorder described herein (e.g., a cardiac disease or a cardiac disorder). The specific therapeutically effective dose level for any particular patient will depend upon a variety of factors including the disorder being treated and the severity of the disorder; the the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof employed; the disclosed methods employed; the age, body weight, general health, sex and diet of the patient; the time of administration; the route of administration; the rate of excretion of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof employed; the duration of the treatment; drugs used in combination or coincidental with the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof employed, and other like factors well known in the medical arts. For example, it is well within the skill of the art to start doses of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a cardiac disease or a cardiac disorder.

In an aspect, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqucous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their case of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In an aspect, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, the term “package insert” is used to refer to instructions customarily included in commercial packages of therapeutic products, that contain information about the indications, usage, dosage, administration, contraindications and/or warnings concerning the use of such therapeutic products.

In an aspect, the term “in combination” in the context of the administration of one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof includes the use of more than one therapy (e.g., additional therapeutic agents). Administration “in combination with” one or more additional therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof, or one or more additional therapeutic agents) to a subject having or diagnosed with a cardiac disease or a cardiac disorder.

In an aspect, “CRISPR or clustered regularly interspaced short palindromic repeat” is an ideal tool for correction of genetic abnormalities associated with diseases such as a cardiac disease or a cardiac disorder. The system can be designed to target genomic DNA directly. In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5c (or CasD), Cas6, Cas6c, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Csc2 (or CasB), Csc3 (or CasE), Csc4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphacra watsonii, Cyanothece sp., Microcystis aeruginosa, Syncchococcus sp., Acetohalobium arabaticum, Ammonifex degensii, Caldicelulosiruptor becscii, Candidatus Desulforudis, Clostridium botulinum, Clostridium difficile, Finegoldia magna, Natranacrobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pscudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Campylobacter jejuni, Oscillatoria sp., Petrotoga mobilis, Thermosipho africanus, or Acaryochloris marina. In an aspect, the CRISPR-based nuclease can be derived from a Cas9 protein from Streptococcus pyogenes. In an aspect, the CRISPR-based nuclease can comprise the sequence set forth in any one of SEQ ID NO:13-SEQ ID NO:22.

In an aspect, “CRISPRa” refers to CRISPR Activation, which is using a dCas9 or dCas9-activator with a gRNA to increase transcription of a target gene.

In an aspect, “CRISPRi” refers to CRISPR Interference, which is using a dCas9 or dCas9-repressor with a gRNA to repress/decrease transcription of a target gene. In an aspect, “dCas9” refers to enzymatically inactive form of Cas9, which can bind, but cannot cleave, DNA. The dCas9, also known as endonuclease deficient Cas9, is a mutant form of Cas9, whose endonuclease activity is removed by mutating the endonuclease domains. However, the dCas9 can still bind to its guide RNA and the DNA strand that is being targeted. The dCas9 can be used in CRISPR interference (CRISPRi) as well as CRISPR activation (CRISPRa). In CRISPRi, dCas9 binds to its DNA target but does not cleave it.

In an aspect, “Protospacer Adjacent Motif” or “PAM” refers to a sequence adjacent to the target sequence that is necessary for Cas enzymes to bind target DNA.

Disclosed are the components to be used to prepare the disclosed nucleic acid molecules, disclosed pharmaceutical formulations, disclosed iPSC-CMs, disclosed plasmics, disclosed vectors, or any combination thereof as well the disclosed isolated nucleic acid molecules, disclosed pharmaceutical formulations, disclosed vectors, or any combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### B. Compositions

### 1. Nucleic Acid Molecules

Disclosed herein is a nucleic acid molecule, comprising: a nucleic acid sequence encoding (i) at least one zinc finger protein, (ii) at least one polypeptide having effector activity, and (iii) a deactivated Cas9 (dCas9) endonuclease. Disclosed herein is a nucleic acid molecule, comprising: a sequence encoding a Zim3-KRAB-dCas9 effector. Disclosed herein is a nucleic acid molecule, comprising: a sequence encoding a Zim3-KRAB-MeCP2-dCas9 effector.

In an aspect, a disclosed encoded zinc finger protein can comprise the sequence set forth in SEQ ID NO:01 or a fragment thereof. In an aspect, a disclosed zinc finger protein can comprise the mRNA sequence set forth in SEQ ID NO:02 or a fragment thereof. In an aspect, a disclosed zinc finger protein can comprise the genomic sequence set forth in SEQ ID NO:03 or a fragment thereof.

In an aspect, a disclosed polypeptide having effector activity can comprise the sequence set forth in SEQ ID NO:23 or a fragment thereof. In an aspect, a disclosed encoded polypeptide having effector activity can comprise the sequence set forth in SEQ ID NO:24 or SEQ ID NO:25 or a fragment thereof. In an aspect, a disclosed polypeptide having effector activity can comprise the sequence set forth in SEQ ID NO:26 or a fragment thereof.

In an aspect, a disclosed deactivated Cas (dCas9) endonuclease can comprise the sequence set forth in any one of SEQ ID NO:13-SEQ ID NO:19 or a fragment thereof. In an aspect, a disclosed encoded deactivated Cas (dCas9) endonuclease can comprise the sequence set forth in any one of SEQ ID NO:20-SEQ ID NO:22 or a fragment thereof.

In an aspect, a disclosed effector activity can comprise transcription activation activity or transcription repression activity. In an aspect, a disclosed effector activity can be paradoxical. For example, if expected to repress transcription, then in a paradoxical aspect, a disclosed effector can activate transcription. If expected to activate transcription, then in a paradoxical aspect, a disclosed effector can repress transcription. In an aspect, a disclosed effector can demonstrate an increased expression and/or activity level when compared to wild-type or control expression level. In an aspect, a disclosed effector can demonstrate a decreased expression and/or activity level when compared to wild-type or control expression level.

In an aspect, a disclosed nucleic acid molecule can be isolated and/or purified.

In an aspect, a disclosed zinc finger protein can comprise Zinc Finger Imprinted 3 (Zim3). In an aspect, Zim3 can also be called by or known as ZFP654 and ZFP264. In an aspect, a disclosed zinc finger protein can comprise Zim3/ZNF657/ZNF264. In an aspect, Zim3 can be described by HGNC Ref. No. 16366, NCBI Gene No. 114026, Ensembl Ref. No. ENSG00000141946, UniProtKB/Swiss-Prot Ref. No. Q96PE6, or any combination thereof. In an aspect, Zim3 (Zinc Finger Imprinted 3) is a Protein Coding gene. In an aspect, Zim3 can enable DNA-binding transcription factor activity, RNA polymerase II-specific and RNA polymerase II cis-regulatory region sequence-specific DNA binding activity, can regulate transcription by RNA polymerase II, can be active in nucleus, or any combination thereof.

In an aspect, a disclosed encoded Zim3 can comprise the sequence set forth in SEQ ID NO:01 or a fragment thereof. In an aspect, a disclosed Zim3 can comprise the mRNA sequence set forth in SEQ ID NO:02 or a fragment thereof. In an aspect, a disclosed Zim3 can comprise the genomic sequence set forth in SEQ ID NO:03 or a fragment thereof. In an aspect, a disclosed encoded Zim3 can comprise a sequence having at least 50%, at least 60%, at least 70%, at least 80%, at least 90%, or more than 90% identity to the sequence set forth in SEQ ID NO:01. In an aspect, a disclosed Zim3 can comprise a mRNA sequence having at least 50%, at least 60%, at least 70%, at least 80%, at least 90%, or more than 90% identity to the sequence set forth in SEQ ID NO:02. In an aspect, a disclosed Zim3 can comprise a genomic sequence having at least 50%, at least 60%, at least 70%, at least 80%, at least 90%, or more than 90% identity to the sequence set forth in SEQ ID NO:03.

In an aspect, a disclosed dCas endonuclease can comprise a dCas9 endonuclease. In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER. In an aspect, a disclosed dCas9 can comprise a catalytically dead mutant of the Cas9 endonuclease from the Streptococcus pyogenes Type II CRISPR/Cas system. In an aspect, a disclosed dCas9 can comprise a catalytically dead mutant of the Cas9 endonuclease from the Staphylococcus aureus Type II CRISPR/Cas system. In an aspect, a disclosed dCas9 can comprise a catalytically dead mutant of the Cas9 endonuclease from the Campylobacter jejuni Type II CRISPR/Cas system.

In an aspect, a disclosed dSpCas9 can comprise the sequence set forth in SEQ ID NO:13 or SEQ ID NO:14 or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:13 or SEQ ID NO:14. In an aspect, a disclosed encoded dSpCas9 can comprise the sequence set forth in SEQ ID NO:20 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect, a disclosed dSaCas9 can comprise the sequence set forth in SEQ ID NO:15 or SEQ ID NO:16 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:15 or SEQ ID NO:16 or a fragment thereof. In an aspect, a disclosed encoded dSaCas9 can comprise the sequence set forth in SEQ ID NO:21 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dCjCas9 can comprise the sequence set forth in SEQ ID NO:17 or SEQ ID NO:18 or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:17 or SEQ ID NO:18 or a fragment thereof. In an aspect, a disclosed encoded dCjCas9 can comprise the sequence set forth in SEQ ID NO:22 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:22 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:19.

In an aspect, a disclosed encoded polypeptide having effector activity can comprise transcription activation activity or transcription repression activity. In an aspect, a disclosed effector activity can comprise transcription activation activity or transcription repression activity. In an aspect, a disclosed effector activity can be paradoxical. For example, if expected to repress transcription, then in a paradoxical aspect, a disclosed effector can activate transcription. If expected to activate transcription, then in a paradoxical aspect, a disclosed effector can repress transcription.

In an aspect, a disclosed polypeptide having effector activity can comprise KRAB or KRAB-MeCP2. In an aspect, KRAB refers to Krüppel-Associated Box. In an aspect, KRAB-MeCP2 refers to Krüppel-Associated Box and Methyl-CpG Binding Protein 2 (MeCP2). In an aspect, a disclosed KRAB can comprise the sequence set forth in SEQ ID NO:23 or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:23 or a fragment thereof. In an aspect, a disclosed encoded KRAB can comprise the sequence set forth in SEQ ID NO:24 or SEQ ID NO:25 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:24 or SEQ ID NO:25 or a fragment thereof.

In an aspect, a disclosed KRAB-MeCP2 can comprise the sequence set forth in SEQ ID NO:26 or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:26 or a fragment thereof. In an aspect, a disclosed encoded KRAB-MeCP2 can comprise the sequence set forth in SEQ ID NO:27 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:27 or a fragment thereof.

In an aspect, a disclosed encoded dCas9 endonuclease is fused to the at least one encoded polypeptide having effector activity (e.g., KRAB or KRAB-MeCP2). In an aspect, a disclosed dCas9 endonuclease can comprise dSpCas9 and a disclosed encoded polypeptide can comprise KRAB or KRAB-MeCP2. In an aspect, a disclosed dCas9 endonuclease can comprise dSaCas9 and a disclosed encoded polypeptide can comprise KRAB or KRAB-MeCP2. In an aspect, a disclosed dCas9 endonuclease can comprise dCjCas9 and a disclosed polypeptide can comprise KRAB or KRAB-MeCP2. In an aspect, a disclosed dCas9 endonuclease can comprise dVQR, dEQR, or dVRER and a disclosed encoded polypeptide can comprise KRAB or KRAB-MeCP2. In an aspect, a disclosed dCas9 endonuclease can comprise dSpCas9, dSaCas9, or dCjCas9 and a disclosed encoded polypeptide can comprise KRAB or KRAB-MeCP2. In an aspect, a disclosed dCas9 endonuclease can comprise dSpCas9, dSaCas9, or dCjCas9, and KRAB or KRAB-MeCP2. In an aspect, a disclosed dCas9 and a disclosed KRAB-McCP2 can comprise the sequence set forth in SEQ ID NO:28.

In an aspect, a disclosed encoded Zim3, a disclosed encoded polypeptide having effector activity, and a disclosed dCas9 endonuclease can be joined. In an aspect, a disclosed encoded Zim3, a disclosed encoded KRAB or KRAB-McCP2, and a disclosed dCas9 endonuclease can be joined. In an aspect, a disclosed encoded Zim3, a disclosed encoded KRAB or KRAB-McCP2, and a disclosed dSpCas9 endonuclease can be joined. In an aspect, a disclosed encoded Zim3, a disclosed encoded KRAB or KRAB-McCP2, and a disclosed dSaCas9 endonuclease can be joined. In an aspect, a disclosed encoded Zim3, a disclosed encoded KRAB or KRAB-McCP2, and a disclosed dCjCas9 endonuclease can be joined. In an aspect, a disclosed dCas9 endonuclease can comprise dVQR, dEQR, or dVRER and a disclosed encoded polypeptide can comprise KRAB or KRAB-MeCP2. In an aspect, a disclosed dCas9 endonuclease can comprise dSpCas9, dSaCas9, or dCjCas9 and a disclosed encoded polypeptide can comprise KRAB or KRAB-MeCP2. In an aspect, a disclosed zinc finger protein can comprise Zim3. In an aspect, a disclosed nucleic acid sequence can comprise Zim3-KRAB-dCas9. In an aspect, a disclosed nucleic acid sequence can comprise Zim3-KRAB-MeCP2-dCas9. In an aspect, a disclosed nucleic acid sequence can encode Zim3-KRAB-dCas9. In an aspect, a disclosed nucleic acid sequence can encode Zim3-KRAB-MeCP2-dCas9.

In an aspect, Zim3-KRAB-dCas9 can effect transcription activation activity. In an aspect, Zim3-KRAB-dCas9 can effect transcription repression activity. In an aspect, Zim3-KRAB-dCas9 can effect transcription activation activity, which can be paradoxical.

In an aspect, a disclosed nucleic acid sequence can encode the sequence set forth in any one of SEQ ID NO:24, SEQ ID NO:25, SEQ ID NO:27, or SEQ ID NO:28, and can encode the sequence set forth in any one of SEQ ID NO:20-SEQ ID NO:22. In an aspect, a disclosed nucleic acid sequence can comprise the sequence set forth in SEQ ID NO:23 and the sequence set forth in SEQ ID NO:14.

In an aspect, a disclosed effector can upregulate the expression of one or more ion channels. In an aspect, a disclosed effector can upregulate the expression of one or more cardiac ion channels. In an aspect, a disclosed effector can upregulate the expression of one or more KCNJ2, KCNH2, GJA1, or any combination thereof.

In an aspect, a disclosed ion channel can be potassium inwardly rectifying channel subfamily J member 2 (KCNJ2). In an aspect, KCNJ2 can be described by HGNC Ref. No. 6263, NCBI Gene No. 3759, Ensembl Ref. No. ENSG00000123700, OMIM Ref. No. 600681, UniProtKB/Swiss-Prot Ref. No. P63252, or any combination thereof. In an aspect, KCNJ2 can be associated with Andersen Cardiodysrhythmic Periodic Paralysis and Short Qt Syndrome 3. In an aspect, a disclosed encoded KCNJ2 can comprise the sequence set forth in SEQ ID NO:04 or a fragment thereof. In an aspect, a disclosed KCNJ2 can comprise the mRNA sequence set forth in SEQ ID NO:05 or a fragment thereof. In an aspect, a disclosed KCNJ2 can comprise the genomic sequence set forth in SEQ ID NO:06 or a fragment thereof. In an aspect, a disclosed encoded KCNJ2 can comprise a sequence having at least 50%, at least 60%, at least 70%, at least 80%, at least 90%, or more than 90% identity to the sequence set forth in SEQ ID NO:04. In an aspect, a disclosed KCNJ2 can comprise a mRNA sequence having at least 50%, at least 60%, at least 70%, at least 80%, at least 90%, or more than 90% identity to the sequence set forth in SEQ ID NO:05. In an aspect, a disclosed KCNJ2 can comprise a genomic sequence having at least 50%, at least 60%, at least 70%, at least 80%, at least 90%, or more than 90% identity to the sequence set forth in SEQ ID NO:06.

In an aspect, a disclosed ion channel can be potassium voltage-gated channel subfamily H member 2 (KCNH2). In an aspect, KCNH2 can be described by HGNC Ref. No. 6251, NCBI Gene No. 3757, Ensembl Ref. No. ENSG00000055118, OMIM Ref. No. 152427, UniProtKB/Swiss-Prot Ref. No. Q12809, or any combination thereof. In an aspect, KCNH2 can refer to a voltage-activated potassium channel found in cardiac muscle, nerve cells, and microglia. In an aspect, 4 copies of this protein interact with one copy of the KCNE2 protein to form a functional potassium channel. In an aspect, a disclosed mutation in this gene encoding KCNH2 can cause long QT syndrome type 2 (LQT2) and Short Qt Syndrome 1. In an aspect, a disclosed encoded KCNH2 can comprise the sequence set forth in SEQ ID NO:07 or a fragment thereof. In an aspect, a disclosed KCNH2 can comprise the mRNA sequence set forth in SEQ ID NO:08 or a fragment thereof. In an aspect, a disclosed KCNH2 can comprise the genomic sequence set forth in SEQ ID NO:09 or a fragment thereof. In an aspect, a disclosed encoded KCNH2 can comprise a sequence having at least 50%, at least 60%, at least 70%, at least 80%, at least 90%, or more than 90% identity to the sequence set forth in SEQ ID NO:07. In an aspect, a disclosed KCNH2 can comprise a mRNA sequence having at least 50%, at least 60%, at least 70%, at least 80%, at least 90%, or more than 90% identity to the sequence set forth in SEQ ID NO:08. In an aspect, a disclosed KCNH2 can comprise a genomic sequence having at least 50%, at least 60%, at least 70%, at least 80%, at least 90%, or more than 90% identity to the sequence set forth in SEQ ID NO:09.

In an aspect, a disclosed ion channel can be gap junction protein alpha 1 (GJA1). In an aspect, GJA1 can be described by HGNC Ref. No. 4274, NCBI Gene No. 2697, Ensembl Rcf. No. ENSG00000152661, OMIM Ref. No. 121014, UniProtKB/Swiss-Prot Ref. No. P17302, or any combination thereof. In an aspect, GJA1 can be associated with gap junctions, which are composed of arrays of intercellular channels that provide a route for the diffusion of low molecular weight materials from cell to cell. In an aspect, the encoded protein GJA1 can be the major protein of gap junctions in the heart, having a preeminent role in the synchronized contraction of the heart and in embryonic development. In an aspect, GJA1 can be associated with Oculodentodigital Dysplasia and Syndactyly, Type Iii. In an aspect, GJA1 can be a main gap junction protein in ventricular cardiomyocytes. In an aspect, a disclosed encoded GJA1 can comprise the sequence set forth in SEQ ID NO:10 or a fragment thereof. In an aspect, a disclosed GJA1 can comprise the mRNA sequence set forth in SEQ ID NO:11 or a fragment thereof. In an aspect, a disclosed GJA1 can comprise the genomic sequence set forth in SEQ ID NO:12 or a fragment thereof. In an aspect, a disclosed encoded GJA1 can comprise a sequence having at least 50%, at least 60%, at least 70%, at least 80%, at least 90%, or more than 90% identity to the sequence set forth in SEQ ID NO:10. In an aspect, a disclosed GJA1 can comprise a mRNA sequence having at least 50%, at least 60%, at least 70%, at least 80%, at least 90%, or more than 90% identity to the sequence set forth in SEQ ID NO:11. In an aspect, a disclosed GJA1 can comprise a genomic sequence having at least 50%, at least 60%, at least 70%, at least 80%, at least 90%, or more than 90% identity to the sequence set forth in SEQ ID NO:12.

In an aspect, a disclosed isolated nucleic acid molecule can comprise a gRNA targeting one or more cardiac ion channels (e.g., for example, KCNJ2, KCNH2, GJA1, or any combination thereof). In an aspect, a disclosed isolated nucleic acid molecule does not comprise a gRNA targeting one or more cardiac ion channels. In an aspect, a disclosed isolated nucleic acid molecule can exclude comprise a gRNA targeting one or more cardiac ion channels.

In an aspect, a disclosed isolated nucleic acid molecule can further comprise a nucleic acid sequencing encoding one or more regulatory elements and/or one or more elements that contribute to and/or confer stabilization of a resulting transcript. In an aspect, a disclosed regulatory element can comprise a promoter, an enhancer, a promoter/enhancer, an internal ribosomal entry site, a transcription terminal signal, a polyadenylation signal, a p2A signal, a woodchuck hepatitis virus post-transcriptional regulatory element (WPRE), a Phi signal-packaging signal (see, e.g., SEQ ID NO:42), a rev responsive element, a 5′-LTR (see, e.g., SEQ ID NO:40), a 3′-LTR (see, e.g., SEQ ID NO:41), an inverted terminal repeat, a nuclear localization signal (NLS), or any combination thereof.

In an aspect, a disclosed isolated nucleic acid molecule can further comprise a nucleic acid sequencing encoding one or more reporters (such as, for example, a fluorescent protein like GFP or mCherry). In an aspect, a disclosed NLS can comprise the sequence set forth in any one of SEQ ID NO:31-SEQ ID NO:37 or a fragment thereof. In an aspect, a disclosed PolyA sequence can comprise the sequence set forth in SEQ ID NO:38 or SEQ ID NO:39 or a fragment thereof. In an aspect, a disclosed ITR can comprise the sequence set forth in SEQ ID NO:40 or SEQ ID NO:41 or a fragment thereof.

In an aspect, a disclosed isolated nucleic acid molecule can further a nucleic acid sequence encoding one or more promoters. In an aspect, a disclosed promoter can comprise a SV40 promoter, a U6 promoter, a chicken β-actin promoter, an EF-1α, a CMV promoter, a CMV promoter/enhancer, a fragment thereof, a SFFV promoter, or any combination thereof.

In an aspect, a disclosed SFFV promoter can comprise the sequence set forth in SEQ ID NO:43 or SEQ ID NO:44 or a fragment thereof. In an aspect, a disclosed SFFV promoter can comprise a sequence having at least 60%, at least 65%, at least 70%, at least 75%, at least 80%, at least 85%, at least 90%, at least 95%, or greater than 95% identity to the sequence set forth in SEQ ID NO:43 or SEQ ID NO:44 or a fragment thereof.

In an aspect, a disclosed U6 promoter can comprise the sequence set forth in SEQ ID NO:48 or a fragment thereof. In an aspect, a disclosed U6 promoter can comprise a sequence having at least 60%, at least 65%, at least 70%, at least 75%, at least 80%, at least 85%, at least 90%, at least 95%, or greater than 95% identity to the sequence set forth in SEQ ID NO:48 or a fragment thereof.

In an aspect, a disclosed EF-1α promoter can comprise the sequence set forth in SEQ ID NO:46, SEQ ID NO:47, or a fragment thereof. In an aspect, a disclosed EF-1α promoter can comprise a sequence having at least 60%, at least 65%, at least 70%, at least 75%, at least 80%, at least 85%, at least 90%, at least 95%, or greater than 95% identity to the sequence set forth in SEQ ID NO:46, SEQ ID NO:47, or a fragment thereof. In an aspect, a promoter can be a short EF1α promoter.

In an aspect, a disclosed promoter can be operably linked to the dCas endonuclease. In an aspect, a disclosed promoter operably linked to the dCas endonuclease can comprise a disclosed SFFV promoter. In an aspect, a disclosed promoter operably linked to the dCas endonuclease can be operably linked to the at least one polypeptide having repressor activity. In an aspect, a disclosed promoter can be operably linked to the dCas endonuclease and the at least one polypeptide having effector activity. In an aspect, a disclosed promoter can be operably linked to a disclosed Zim3-KRAB-dCas9 effector. In an aspect, a disclosed promoter operably can be linked to the at least one guide RNA targeting a gene of interest or portion thereof. In an aspect, a disclosed promoter operably linked to the at least one guide RNA can comprise a U6 promoter. In an aspect, a disclosed isolated nucleic acid molecule can further comprise a gRNA scaffold.

In an aspect, a disclosed isolated nucleic acid molecule can further comprise a nucleic acid sequence encoding one or more promoters, wherein a first promoter can be operably linked to the dCas endonuclease, and wherein a second promoter can be operably linked to the at least one guide RNA targeting a gene of interest or portion thereof. In an aspect, a disclosed isolated nucleic acid molecule can further comprise a nucleic acid sequence encoding one or more promoters, wherein a first promoter can be operably linked to the dCas9 endonuclease and the at least one polypeptide having an repressor activity, and wherein a second promoter can be operably linked to the at least one guide RNA targeting a gene of interest thereof.

In an aspect, a disclosed nucleic acid sequence can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art. In an aspect, any disclosed Cas9 endonuclease, a disclosed polypeptide having enzymatic activities, a disclosed fusion product or a disclosed fusion protein, or any combination thereof can be codon-optimized.

One problem of iPSC-CM that are differentiated using commonly used techniques can be that they are stuck at a relatively immature stage of differentiation with a morphology and marker expression that is very similar to a cardiomyocyte in the early embryonic heart. A disclosed nucleic acid molecule can improve or enhance maturation of iPSC-CMs.

In an aspect, a disclosed nucleic acid molecule can demonstrate an increased expression and/or activity level when compared to a wild-type expression level or control expression level, or when compared to the expression level in the absence of the disclosed nucleic acid molecule. In an aspect, disclosed nucleic acid molecule can demonstrate a decreased expression and/or activity level when compared to a wild-type expression level or control expression level, or when compared to the expression level in the absence of the disclosed nucleic acid molecule.

In an aspect, a disclosed nucleic acid molecule can increase the expression and/or activity level of one or more cardiac ion channels when compared to a wild-type expression level or control expression level, or when compared to the expression level in the absence of the disclosed nucleic acid molecule. In an aspect, a disclosed nucleic acid molecule can increase the expression level and/or activity level of one or more of KCNJ2, KCNH2, GJA1, or any combination thereof, or when compared to the expression level in the absence of the disclosed nucleic acid molecule.

In an aspect, a disclosed nucleic acid molecule can enhance or improve the functionality of one or more cardiac ion channels when compared to a wild-type expression level or control expression level, or when compared to the expression level in the absence of the disclosed nucleic acid molecule. In an aspect, a disclosed nucleic acid molecule can enhance or improve the functionality of KCNJ2, KCNH2, GJA1, or any combination thereof, or when compared to the expression level in the absence of the disclosed nucleic acid molecule.

In an aspect, a disclosed nucleic acid molecule can improve or enhance the functionality of one or more cardiac ion channels when compared to a wild-type expression level or control expression level, or when compared to the expression level in the absence of the disclosed nucleic acid molecule. In an aspect, a disclosed nucleic acid molecule can improve or enhance the functionality of KCNJ2, KCNH2, GJA1, or any combination thereof, or when compared to the expression level in the absence of the disclosed nucleic acid molecule.

In an aspect, a disclosed nucleic acid molecule can drive or stimulate one or more electrophysiological changes indicative of a mature phenotype in iPSC-CMs when compared to a wild-type expression level or control expression level, or when compared to the expression level in the absence of the disclosed nucleic acid molecule. In an aspect, a disclosed nucleic acid molecule can drive or stimulate one or more electrophysiological changes (e.g., increase in expression of KCNJ2, KCNH2, GJA1, or any combination thereof) indicative of a mature phenotype in iPSC-CMs, or when compared to the expression level in the absence of the disclosed nucleic acid molecule.

In an aspect, matured iPSC-CMs can be characterized by mitochondrial maturation, increased oxidative capacity, and enhanced fatty acid use for energy production. For example, the structural, electrophysiological, contractile, and metabolic characteristics of iPSC-CMs can be under-developed when compared to adulty cardiomyocytes. In an aspect, a disclosed nucleic acid molecule can be used to generate a mature structural, electrophysiological, contractile, and metabolic profile.

In an aspect, maturation of iPSC-CMs improved or enhanced by a disclosed nucleic acid molecule can be assessed by an increase in the expression level of PDK4, CD36, PPARA, ATP5, LPL, SCD, PPARD, ACADVL, ACAT1, DGAT1, PPARGC1A, ESRRA, N2B, CAV3, SERCA2, CPT1A/1B, or any combination thereof. In an aspect, maturation of iPSC-CMs improved or enhanced by a disclosed nucleic acid molecule can be assessed by a decrease in the expression level of ALDOA, HK1, HK2, PGK1, GAPDH, LDHA, or any combination thereof.

In an aspect, maturation of iPSC-CMs improved or enhanced by a disclosed nucleic acid molecule can be assessed by an increase in mitochondrial numbers and size, an increase in the number of peri-sarcomeric mitochondria, a decrease in the number of perinuclear mitochondria, or any combination thereof.

In an aspect, maturation of iPSC-CMs improved or enhanced by a disclosed nucleic acid molecule can be assessed by an increase in the expression of genes encoding electronic transport chain (ETC) proteins. In an aspect, maturation of iPSC-CMs improved or enhanced by a disclosed nucleic acid molecule can be assessed by a reduction in glucose uptake, glycogen storage, lactate production, hexokinase activity, the proportion of glycolysis-related ATP production, or any combination thereof. In an aspect, maturation of iPSC-CMs improved or enhanced by a disclosed nucleic acid molecule can be assessed by an increase in the relative contribution of fatty acid oxidation (FAO) to ATP production. In an aspect, a disclosed nucleic acid molecule can be used to enhance or increase or promoter the metabolic maturation of iPSC-CMs (e.g., improved mitochondrial structure and function, decreased glycolytic activity, increased FAO, increased ATP production, or any combination thereof).

In an aspect, a disclosed nucleic acid molecule can be used to enhance or increase or promote maturation of iPSC-CMs, which can be used to investigate cardiomyopathy disease mechanisms or can be used to interrogate cardiac disease modeling or can be used to screen drugs for efficacy and/or safety. For example, a drug can be screened against cardiomyocytes and/or cardiac organoids to determine general cardiotoxicity, or to determine whether cardiomyocytes and/or cardiac organoids obtained from progenitor cells of a particular individual display sensitivity to possible cardiotoxic drugs or other molecules or compounds.

In an aspect, a disclosed nucleic acid molecule can be used to enhance or increase or promote maturation of iPSC-CMs as measured and/or assessed by contractile force, morphology, electrophysiology, calcium handling characteristics, metabolic profile, or any combination thereof.

In an aspect, maturation of iPSC-CMs enhanced or increased or promoted by a disclosed nucleic acid molecule can be assessed by beating when stimulated with a force closer to around 40-80 mN/mm² (e.g., indicative of mature CMs) rather than a force closer to around 0.08-4 mN/mm² (e.g., indicative of immature hiPSC-CMs). In an aspect, maturation of iPSC-CMs enhanced or increased or promoted by a disclosed nucleic acid molecule can be assessed by a conduction velocity around 60 cm/s (e.g., indicative of mature CMs) rather than a conduction velocity closer to around 10-20 cm/s (e.g., indicative of immature hiPSC-CMs). In an aspect, maturation of iPSC-CMs enhanced or increased or promoted by a disclosed nucleic acid molecule can be assessed by beating when stimulated with an upstroke velocity about 150-350 V/s (e.g., indicative of mature CMs) rather than an upstroke velocity closer to around 10-50 V/s (e.g., indicative of immature hiPSC-CMs).

In an aspect, a disclosed Zim3-KRAB-dCas9 effector can improve or enhancing maturation of iPSC-CMs. In an aspect, a disclosed Zim3-KRAB-dCas9 effector can demonstrate an increased expression and/or activity level when compared to a wild-type expression level or control expression level, or when compared to the expression level in the absence of the disclosed Zim3-KRAB-dCas9 effector. In an aspect, disclosed Zim3-KRAB-dCas9 effector can demonstrate a decreased expression and/or activity level when compared to a wild-type expression level or control expression level, or when compared to the expression level in the absence of the disclosed Zim3-KRAB-dCas9 effector.

In an aspect, a disclosed Zim3-KRAB-dCas9 effector can increase the expression and/or activity level of one or more cardiac ion channels when compared to a wild-type expression level or control expression level, or when compared to the expression level in the absence of the disclosed Zim3-KRAB-dCas9 effector. In an aspect, a disclosed Zim3-KRAB-dCas9 effector can increase the expression level and/or activity level of one or more of KCNJ2, KCNH2, GJA1, or any combination thereof, or when compared to the expression level in the absence of the disclosed Zim3-KRAB-dCas9 effector.

In an aspect, a disclosed Zim3-KRAB-dCas9 effector can enhance or improve the functionality of one or more cardiac ion channels when compared to a wild-type expression level or control expression level, or when compared to the expression level in the absence of the disclosed Zim3-KRAB-dCas9 effector. In an aspect, a disclosed Zim3-KRAB-dCas9 effector can enhance or improve the functionality of KCNJ2, KCNH2, GJA1, or any combination thereof, or when compared to the expression level in the absence of the disclosed Zim3-KRAB-dCas9 effector.

In an aspect, a disclosed Zim3-KRAB-dCas9 effector can improve or enhance the functionality of one or more cardiac ion channels when compared to a wild-type expression level or control expression level, or when compared to the expression level in the absence of the disclosed Zim3-KRAB-dCas9 effector. In an aspect, a disclosed Zim3-KRAB-dCas9 effector can improve or enhance the functionality of KCNJ2, KCNH2, GJA1, or any combination thereof, or when compared to the expression level in the absence of the disclosed Zim3-KRAB-dCas9 effector.

In an aspect, a disclosed Zim3-KRAB-dCas9 effector can drive or stimulate one or more electrophysiological changes indicative of a mature phenotype in iPSC-CMs when compared to a wild-type expression level or control expression level, or when compared to the expression level in the absence of the disclosed Zim3-KRAB-dCas9 effector. In an aspect, a disclosed Zim3-KRAB-dCas9 effector can drive or stimulate one or more electrophysiological changes (e.g., increase in expression of KCNJ2, KCNH2, GJA1, or any combination thereof) indicative of a mature phenotype in iPSC-CMs, or when compared to the expression level in the absence of the disclosed Zim3-KRAB-dCas9 effector.

In an aspect, matured iPSC-CMs can be characterized by mitochondrial maturation, increased oxidative capacity, and enhanced fatty acid use for energy production. For example, the structural, electrophysiological, contractile, and metabolic characteristics of iPSC-CMs can be under-developed when compared to adulty cardiomyocytes. In an aspect, a disclosed Zim3-KRAB-dCas9 effector can be used to generate a mature structural, electrophysiological, contractile, and metabolic profile.

In an aspect, maturation of iPSC-CMs improved or enhanced by a disclosed Zim3-KRAB-dCas9 effector can be assessed by an increase in the expression level of PDK4, CD36, PPARA, ATP5, LPL, SCD, PPARD, ACADVL, ACAT1, DGAT1, PPARGC1A, ESRRA, CPT1A/1B, or any combination thereof. In an aspect, maturation of iPSC-CMs improved or enhanced by a disclosed Zim3-KRAB-dCas9 effector can be assessed by a decrease in the expression level of ALDOA, HK1, HK2, PGK1, GAPDH, LDHA, or any combination thereof.

In an aspect, maturation of iPSC-CMs improved or enhanced by a disclosed Zim3-KRAB-dCas9 effector can be assessed by an increase in mitochondrial numbers and size, an increase in the number of peri-sarcomeric mitochondria, a decrease in the number of perinuclear mitochondria, or any combination thereof.

In an aspect, maturation of iPSC-CMs improved or enhanced by a disclosed Zim3-KRAB-dCas9 effector can be assessed by an increase in the expression of genes encoding electronic transport chain (ETC) proteins. In an aspect, maturation of iPSC-CMs improved or enhanced by a disclosed Zim3-KRAB-dCas9 effector can be assessed by a reduction in glucose uptake, glycogen storage, lactate production, hexokinase activity, the proportion of glycolysis-related ATP production, or any combination thereof.

In an aspect, maturation of iPSC-CMs improved or enhanced by a disclosed Zim3-KRAB-dCas9 effector can be assessed by an increase in the relative contribution of fatty acid oxidation (FAO) to ATP production. In an aspect, a disclosed Zim3-KRAB-dCas9 effector can be used to enhance or increase or promoter the metabolic maturation of iPSC-CMs (e.g., improved mitochondrial structure and function, decreased glycolytic activity, increased FAO, increased ATP production, or any combination thereof).

In an aspect, a disclosed Zim3-KRAB-dCas9 effector can be used to enhance or increase or promote maturation of iPSC-CMs, which can be used to investigate cardiomyopathy disease mechanisms or can be used to interrogate cardiac disease modeling or can be used to screen drugs for efficacy and/or safety. For example, a drug can be screened against cardiomyocytes and/or cardiac organoids to determine general cardiotoxicity, or to determine whether cardiomyocytes and/or cardiac organoids obtained from progenitor cells of a particular individual display sensitivity to possible cardiotoxic drugs or other molecules or compounds.

In an aspect, a disclosed Zim3-KRAB-dCas9 effector can be used to enhance or increase or promote maturation of iPSC-CMs as measured and/or assessed by contractile force, morphology, electrophysiology, calcium handling characteristics, metabolic profile, or any combination thereof.

In an aspect, maturation of iPSC-CMs enhanced or increased or promoted by a disclosed Zim3-KRAB-dCas9 effector can be assessed by beating when stimulated with a force closer to around 40-80 mN/mm² (e.g., indicative of mature CMs) rather than a force closer to around 0.08-4 mN/mm² (e.g., indicative of immature hiPSC-CMs). In an aspect, maturation of iPSC-CMs enhanced or increased or promoted by a disclosed Zim3-KRAB-dCas9 effector can be assessed by a conduction velocity around 60 cm/s (e.g., indicative of mature CMs) rather than a conduction velocity closer to around 10-20 cm/s (e.g., indicative of immature hiPSC-CMs). In an aspect, maturation of iPSC-CMs enhanced or increased or promoted by a disclosed Zim3-KRAB-dCas9 effector can be assessed by beating when stimulated with an upstroke velocity about 150-350 V/s (e.g., indicative of mature CMs) rather than an upstroke velocity closer to around 10-50 V/s (e.g., indicative of immature hiPSC-CMs).

In an aspect of maturation, iPSC-CMs can be cultured according to one or more methods known to the art. For example, in an aspect, it can be good practice to observe iPSC lines daily under phase contrast microscopy (e.g., 4×, 10×, 20× and 40× magnification) to check for iPSC-like morphology, the presence of differentiated cells and confluence. A typical scoring can be outlined as (i) optimal, compacted iPSC colonies with defined edges; morphology uniform across colonies, (ii) acceptable iPSC colonies with some differentiation around the edges, cells more loosely packed within colonies, (iii) good adherence with small iPSCs colonies emerging, and (iv) poor adherence and no obvious iPSCs. In an aspect, cells can be fed by removing ˜95% of the medium from the wells using an aspirator pipette. In an aspect, the medium cannot be removed completely but rather a thin film of medium can cover the cell layer to avoid drying out the cells. In an aspect, about 2 mL of fresh medium per 1 well of a 6-well plate can be aseptically added by gently adding to the side of the well. Incubate cells at 37° C./5% CO2. In an aspect, a medium exchange can occur daily on six of seven days with increased volume of media (1.5×-2× the normal amount; cell density dependent) if cells need to be left for longer periods between media change. In an aspect, medium exchanges can be effected within 48 hours.

In an aspect, a disclosed nucleic acid molecule can be administered to a subject in need thereof. In an aspect, a disclosed subject can have one or more cardiac diseases or disorders. In an aspect, a disclosed cardiac disease or disorder can comprise myocardial infarction or ischemic cardiomyopathy. In an aspect, a disclosed cardiac disease or disorder can comprise heart failure. In an aspect, a disclosed cardiac disease or disorder can comprise coronary artery disease or an arrhythmia. Cardiac diseases and disorders are known to the skilled person in the art. In an aspect, a disclosed nucleic acid molecule can be added to an implantable cardiac patch. Cardiac patches are known to the art. In an aspect, a disclosed implantable cardiac patch can be provided to a subject in need thereof.

In an aspect, a disclosed nucleic acid molecule can slow or prevent progression of a cardiac disease or disorder in a subject in need thereof. In an aspect, slowing or preventing disease progression or a cardiac disease or disorder can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cardiac disease or disorder; (vii) reversing, inhibiting, preventing, stabilizing, and/or slowing the rate of progression of a cardiac disease or disorder, or (viii) any combination thereof.

In an aspect, a disclosed nucleic acid molecule can decrease or minimize the intensity and/or duration of an arrhythmic event or the number or frequency of arrhythmic events.

In an aspect, a disclosed nucleic acid molecule can be used to transform iPSc-CMs into matured CMs or more matured CMs when compared to the CMs prior to contact with the disclosed nucleic acid molecule. In an aspect, matured CMs can be used in several applications. For example, CMs matured by a disclosed nucleic acid molecule or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, a disclosed nucleic acid molecule can be used to upregulate the expression of ion channels (e.g., KCNJ2, KCNH2, GJA1, etc.) indicative of adult CMs for use in drug evaluation (e.g., efficacy, toxicity, safety, etc.). In an aspect, the upregulation of ion channel expression in matured CMs can be a useful tool in the development of relevant cardiac therapies. In an aspect, matured CMs can be helpful in ensuring that a drug is non-toxic and does not have a proarrhythmic effects prior to administration to a subject in need thereof. In an aspect, matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stereolithography, inkjet, etc.). In an aspect, matured CMs can be used in a disclosed therapeutic application, for example, an application to treat the disease progression of a subject having a diseased or damaged heart. In an aspect, a disclosed nucleic acid molecule can be used stabilize repolarization, upregulate conduction velocity, stabilize the resting membrane potential, reduce spontaneous firing, or any combination thereof. In an aspect, matured CMs can demonstrate stabilized repolarization, upregulated conduction velocity, stabilized the resting membrane potential, reduced spontaneous firing, or any combination thereof.

### 2. Viral Vectors and Non-Viral Vectors

Disclosed herein is a viral vector comprising a sequence for a disclosed nucleic acid molecule. Disclosed herein is a viral vector comprising a nucleic acid sequence encoding (i) at least one zinc finger protein, (ii) at least one polypeptide having effector activity, and (iii) a deactivated Cas9 (dCas9) endonuclease. Disclosed herein is a viral vector comprising a nucleic acid sequence encoding a Zim3-KRAB-dCas9 effector. Disclosed herein is a viral vector comprising a nucleic acid sequence encoding a Zim3-KRAB-MeCP2-dCas9 effector.

Disclosed herein is a recombinant viral vector comprising a disclosed nucleic acid molecule. Disclosed herein is a recombinant viral vector comprising a nucleic acid sequence encoding (i) at least one zinc finger protein, (ii) at least one polypeptide having effector activity, and (iii) a deactivated Cas9 (dCas9) endonuclease. Disclosed herein is a recombinant viral vector comprising a nucleic acid sequence encoding a Zim3-KRAB-dCas9 effector. Disclosed herein is a recombinant viral vector comprising a nucleic acid sequence encoding a Zim3-KRAB-MeCP2-dCas9 effector.

Disclosed herein is a non-viral vector comprising a nucleic acid sequence encoding (i) at least one zinc finger protein, (ii) at least one polypeptide having effector activity, and (iii) a deactivated Cas9 (dCas9) endonuclease. Disclosed herein is a non-viral vector comprising a nucleic acid sequence encoding a Zim3-KRAB-dCas9 effector. Disclosed herein is a non-viral vector comprising a nucleic acid sequence encoding a Zim3-KRAB-MeCP2-dCas9 effector. Disclosed herein is a recombinant non-viral vector comprising a disclosed nucleic acid molecule.

Disclosed herein is a recombinant non-viral vector comprising a nucleic acid sequence encoding (i) at least one zinc finger protein, (ii) at least one polypeptide having effector activity, and (iii) a deactivated Cas9 (dCas9) endonuclease. Disclosed herein is a recombinant non-viral vector comprising a nucleic acid sequence encoding a Zim3-KRAB-dCas9 effector. Disclosed herein is a recombinant non-viral vector comprising a nucleic acid sequence encoding a Zim3-KRAB-MeCP2-dCas9 effector.

In an aspect of a disclosed vector, a disclosed encoded zinc finger protein can comprise the sequence set forth in SEQ ID NO:01 or a fragment thereof. In an aspect of a disclosed vector, a disclosed zinc finger protein can comprise the mRNA sequence set forth in SEQ ID NO:02 or a fragment thereof. In an aspect of a disclosed vector, a disclosed zinc finger protein can comprise the genomic sequence set forth in SEQ ID NO:03 or a fragment thereof. In an aspect of a disclosed vector, a disclosed polypeptide having effector activity can comprise the sequence set forth in SEQ ID NO:23 or a fragment thereof. In an aspect of a disclosed vector, a disclosed encoded polypeptide having effector activity can comprise the sequence set forth in SEQ ID NO:24 or SEQ ID NO:25 or a fragment thereof. In an aspect of a disclosed vector, a disclosed polypeptide having effector activity can comprise the sequence set forth in SEQ ID NO:26 or a fragment thereof. In an aspect of a disclosed vector, a disclosed deactivated Cas9 (dCas9) endonuclease can comprise the sequence set forth in any one of SEQ ID NO:13-SEQ ID NO:19 or a fragment thereof. In an aspect of a disclosed vector, a disclosed encoded deactivated Cas9 (dCas9) endonuclease can comprise the sequence set forth in any one of SEQ ID NO:20-SEQ ID NO:22 or a fragment thereof.

In an aspect of a disclosed vector, a disclosed effector activity can comprise transcription activation activity or transcription repression activity. In an aspect of a disclosed vector, a disclosed effector activity can be paradoxical. For example, if expected to repress transcription, then in a paradoxical aspect, a disclosed effector can activate transcription. If expected to activate transcription, then in a paradoxical aspect, a disclosed effector can repress transcription.

In an aspect, a disclosed vector can be purified and/or isolated.

In an aspect of a disclosed vector, a disclosed effector can demonstrate an increased expression and/or activity level when compared to wild-type or control expression level. In an aspect of a disclosed vector, a disclosed effector can demonstrate a decreased expression and/or activity level when compared to wild-type or control expression level. In an aspect, a wild-type or control level can be a pre-disease or pre-cardiac disease or a pre-cardiac disorder level.

In an aspect, a disclosed zinc finger protein can comprise Zinc Finger Imprinted 3 (Zim3). IN an aspect, Zim3 can also be called by or known as ZFP654 and ZFP264. In an aspect, a disclosed zinc finger protein can comprise Zim3/ZNF657/ZNF264. In an aspect, Zim3 can be described by HGNC Ref. No. 16366, NCBI Gene No. 114026, Ensembl Ref. No. ENSG00000141946, UniProtKB/Swiss-Prot Ref. No. Q96PE6, or any combination thereof.

In an aspect, Zim3 (Zinc Finger Imprinted 3) is a Protein Coding gene. In an aspect, Zim3 can enable DNA-binding transcription factor activity, RNA polymerase II-specific and RNA polymerase II cis-regulatory region sequence-specific DNA binding activity, can regulate transcription by RNA polymerase II, can be active in nucleus, or any combination thereof.

In an aspect, a disclosed encoded Zim3 can comprise the sequence set forth in SEQ ID NO:01 or a fragment thereof. In an aspect, a disclosed Zim3 can comprise the mRNA sequence set forth in SEQ ID NO:02 or a fragment thereof. In an aspect, a disclosed Zim3 can comprise the genomic sequence set forth in SEQ ID NO:03 or a fragment thereof.

In an aspect, a disclosed encoded Zim3 can comprise a sequence having at least 50%, at least 60%, at least 70%, at least 80%, at least 90%, or more than 90% identity to the sequence set forth in SEQ ID NO:01. In an aspect, a disclosed Zim3 can comprise a mRNA sequence having at least 50%, at least 60%, at least 70%, at least 80%, at least 90%, or more than 90% identity to the sequence set forth in SEQ ID NO:02. In an aspect, a disclosed Zim3 can comprise a genomic sequence having at least 50%, at least 60%, at least 70%, at least 80%, at least 90%, or more than 90% identity to the sequence set forth in SEQ ID NO:03.

In an aspect, a disclosed dCas endonuclease can comprise a dCas9 endonuclease. In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER. In an aspect, a disclosed dCas9 can comprise a catalytically dead mutant of the Cas9 endonuclease from the Streptococcus pyogenes Type II CRISPR/Cas system. In an aspect, a disclosed dCas9 can comprise a catalytically dead mutant of the Cas9 endonuclease from the Staphylococcus aureus Type II CRISPR/Cas system. In an aspect, a disclosed dCas9 can comprise a catalytically dead mutant of the Cas9 endonuclease from the Campylobacter jejuni Type II CRISPR/Cas system.

In an aspect, a disclosed dSpCas9 can comprise the sequence set forth in SEQ ID NO:13 or SEQ ID NO:14 or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:13 or SEQ ID NO:14. In an aspect, a disclosed encoded dSpCas9 can comprise the sequence set forth in SEQ ID NO:20 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect, a disclosed dSaCas9 can comprise the sequence set forth in SEQ ID NO:15 or SEQ ID NO:16 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:15 or SEQ ID NO:16 or a fragment thereof. In an aspect, a disclosed encoded dSaCas9 can comprise the sequence set forth in SEQ ID NO:21 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dCjCas9 can comprise the sequence set forth in SEQ ID NO:17 or SEQ ID NO:18 or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:17 or SEQ ID NO:18 or a fragment thereof. In an aspect, a disclosed encoded dCjCas9 can comprise the sequence set forth in SEQ ID NO:22 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:22 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:19.

In an aspect, a disclosed encoded polypeptide having effector activity can comprise transcription activation activity or transcription repression activity. In an aspect, a disclosed effector activity can comprise transcription activation activity or transcription repression activity. In an aspect, a disclosed effector activity can be paradoxical. For example, if expected to repress transcription, then in a paradoxical aspect, a disclosed effector can activate transcription. If expected to activate transcription, then in a paradoxical aspect, a disclosed effector can repress transcription.

In an aspect, a disclosed polypeptide having effector activity can comprise KRAB or KRAB-MeCP2. In an aspect, KRAB refers to Krüppel-Associated Box. In an aspect, KRAB-MeCP2 refers to Krüppel-Associated Box and Methyl-CpG Binding Protein 2 (MeCP2). In an aspect, a disclosed KRAB can comprise the sequence set forth in SEQ ID NO:23 or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:23 or a fragment thereof. In an aspect, a disclosed encoded KRAB can comprise the sequence set forth in SEQ ID NO:24 or SEQ ID NO:25 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:24 or SEQ ID NO:25 or a fragment thereof. In an aspect, a disclosed KRAB-MeCP2 can comprise the sequence set forth in SEQ ID NO:26 or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:26 or a fragment thereof. In an aspect, a disclosed encoded KRAB-MeCP2 can comprise the sequence set forth in SEQ ID NO:27 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:27 or a fragment thereof.

In an aspect, a disclosed encoded dCas9 endonuclease is fused to the at least one encoded polypeptide having effector activity (e.g., KRAB or KRAB-MeCP2). In an aspect, a disclosed dCas9 endonuclease can comprise dSpCas9 and a disclosed encoded polypeptide can comprise KRAB or KRAB-MeCP2. In an aspect, a disclosed dCas9 endonuclease can comprise dSaCas9 and a disclosed encoded polypeptide can comprise KRAB or KRAB-MeCP2. In an aspect, a disclosed dCas9 endonuclease can comprise dCjCas9 and a disclosed polypeptide can comprise KRAB or KRAB-MeCP2.

In an aspect, a disclosed dCas9 endonuclease can comprise dVQR, dEQR, or dVRER and a disclosed encoded polypeptide can comprise KRAB or KRAB-MeCP2. In an aspect, a disclosed dCas9 endonuclease can comprise dSpCas9, dSaCas9, or dCjCas9 and a disclosed encoded polypeptide can comprise KRAB or KRAB-MeCP2. In an aspect, a disclosed dCas9 endonuclease can comprise dSpCas9, dSaCas9, or dCjCas9, and KRAB or KRAB-MeCP2. In an aspect, a disclosed dCas9 and a disclosed KRAB-MeCP2 can comprise the sequence set forth in SEQ ID NO:28.

In an aspect, a disclosed encoded Zim3, a disclosed encoded polypeptide having effector activity, and a disclosed dCas9 endonuclease can be joined. In an aspect, a disclosed encoded Zim3, a disclosed encoded KRAB or KRAB-McCP2, and a disclosed dCas9 endonuclease can be joined. In an aspect, a disclosed encoded Zim3, a disclosed encoded KRAB or KRAB-McCP2, and a disclosed dSpCas9 endonuclease can be joined. In an aspect, a disclosed encoded Zim3, a disclosed encoded KRAB or KRAB-McCP2, and a disclosed dSaCas9 endonuclease can be joined. In an aspect, a disclosed encoded Zim3, a disclosed encoded KRAB or KRAB-McCP2, and a disclosed dCjCas9 endonuclease can be joined.

In an aspect, a disclosed dCas9 endonuclease can comprise dVQR, dEQR, or dVRER and a disclosed encoded polypeptide can comprise KRAB or KRAB-MeCP2. In an aspect, a disclosed dCas9 endonuclease can comprise dSpCas9, dSaCas9, or dCjCas9 and a disclosed encoded polypeptide can comprise KRAB or KRAB-MeCP2. In an aspect, a disclosed zinc finger protein can comprise Zim3. In an aspect, a disclosed nucleic acid sequence can comprise Zim3-KRAB-dCas9. In an aspect, a disclosed nucleic acid sequence can comprise Zim3-KRAB-MeCP2-dCas9. In an aspect, a disclosed nucleic acid sequence can encode Zim3-KRAB-dCas9. In an aspect, a disclosed nucleic acid sequence can encode Zim3-KRAB-MeCP2-dCas9.

In an aspect, Zim3-KRAB-dCas9 can effect transcription activation activity. In an aspect, Zim3-KRAB-dCas9 can effect transcription repression activity. In an aspect, Zim3-KRAB-dCas9 can effect transcription activation activity, which can be paradoxical.

In an aspect, a disclosed nucleic acid sequence can encode the sequence set forth in any one of SEQ ID NO:24, SEQ ID NO:25, SEQ ID NO:27, or SEQ ID NO:28, and can encode the sequence set forth in any one of SEQ ID NO:20-SEQ ID NO:22. In an aspect, a disclosed nucleic acid sequence can comprise the sequence set forth in SEQ ID NO:23 and the sequence set forth in SEQ ID NO:14.

In an aspect, a disclosed viral vector or a disclosed non-viral vector can upregulate the expression of one or more ion channels. In an aspect, a disclosed viral vector or a disclosed non-viral vector can upregulate the expression of one or more cardiac ion channels. In an aspect, a disclosed viral vector or a disclosed non-viral vector can upregulate the expression of one or more KCNJ2, KCNH2, GJA1, or any combination thereof.

In an aspect, a disclosed ion channel can be Potassium Inwardly Rectifying Channel Subfamily J Member 2 (KCNJ2). In an aspect, KCNJ2 can be described by HGNC Ref. No. 6263, NCBI Gene No. 3759, Ensembl Ref. No. ENSG00000123700, OMIM Ref. No. 600681, UniProtKB/Swiss-Prot Ref. No. P63252, or any combination thereof. In an aspect, KCNJ2 can be associated with Andersen Cardiodysrhythmic Periodic Paralysis and Short Qt Syndrome 3. In an aspect, a disclosed encoded KCNJ2 can comprise the sequence set forth in SEQ ID NO:04 or a fragment thereof. In an aspect, a disclosed KCNJ2 can comprise the mRNA sequence set forth in SEQ ID NO:05 or a fragment thereof. In an aspect, a disclosed KCNJ2 can comprise the genomic sequence set forth in SEQ ID NO:06 or a fragment thereof. In an aspect, a disclosed encoded KCNJ2 can comprise a sequence having at least 50%, at least 60%, at least 70%, at least 80%, at least 90%, or more than 90% identity to the sequence set forth in SEQ ID NO:04. In an aspect, a disclosed KCNJ2 can comprise a mRNA sequence having at least 50%, at least 60%, at least 70%, at least 80%, at least 90%, or more than 90% identity to the sequence set forth in SEQ ID NO:05. In an aspect, a disclosed KCNJ2 can comprise a genomic sequence having at least 50%, at least 60%, at least 70%, at least 80%, at least 90%, or more than 90% identity to the sequence set forth in SEQ ID NO:06.

In an aspect, a disclosed ion channel can be potassium voltage-gated channel subfamily H member 2 (KCNH2). In an aspect, KCNH2 can be described by HGNC Ref. No. 6251, NCBI Gene No. 3757, Ensembl Ref. No. ENSG00000055118, OMIM Ref. No. 152427, UniProtKB/Swiss-Prot Ref. No. Q12809, or any combination thereof. In an aspect, KCNH2 can refer to a voltage-activated potassium channel found in cardiac muscle, nerve cells, and microglia. In an aspect, 4 copies of this protein interact with one copy of the KCNE2 protein to form a functional potassium channel. In an aspect, a disclosed mutation in this gene encoding KCNH2 can cause long QT syndrome type 2 (LQT2) and Short Qt Syndrome 1. In an aspect, a disclosed encoded KCNH2 can comprise the sequence set forth in SEQ ID NO:07 or a fragment thereof. In an aspect, a disclosed KCNH2 can comprise the mRNA sequence set forth in SEQ ID NO:08 or a fragment thereof. In an aspect, a disclosed KCNH2 can comprise the genomic sequence set forth in SEQ ID NO:09 or a fragment thereof. In an aspect, a disclosed encoded KCNH2 can comprise a sequence having at least 50%, at least 60%, at least 70%, at least 80%, at least 90%, or more than 90% identity to the sequence set forth in SEQ ID NO:07. In an aspect, a disclosed KCNH2 can comprise a mRNA sequence having at least 50%, at least 60%, at least 70%, at least 80%, at least 90%, or more than 90% identity to the sequence set forth in SEQ ID NO:08.

In an aspect, a disclosed KCNH2 can comprise a genomic sequence having at least 50%, at least 60%, at least 70%, at least 80%, at least 90%, or more than 90% identity to the sequence set forth in SEQ ID NO:09.

In an aspect, a disclosed ion channel can be gap junction protein alpha 1 (GJA1). In an aspect, GJA1 can be described by HGNC Ref. No. 4274, NCBI Gene No. 2697, Ensembl Ref No. ENSG00000152661, OMIM Ref. No. 121014, UniProtKB/Swiss-Prot Ref. No. P17302, or any combination thereof. In an aspect, GJA1 can be associated with gap junctions, which are composed of arrays of intercellular channels that provide a route for the diffusion of low molecular weight materials from cell to cell. In an aspect, the encoded protein GJA1 can be the major protein of gap junctions in the heart, having a preeminent role in the synchronized contraction of the heart and in embryonic development. In an aspect, GJA1 can be associated with Oculodentodigital Dysplasia and Syndactyly, Type Iii. In an aspect, a disclosed encoded GJA1 can comprise the sequence set forth in SEQ ID NO:10 or a fragment thereof. In an aspect, a disclosed GJA1 can comprise the mRNA sequence set forth in SEQ ID NO:11 or a fragment thereof. In an aspect, a disclosed GJA1 can comprise the genomic sequence set forth in SEQ ID NO:12 or a fragment thereof. In an aspect, a disclosed encoded GJA1 can comprise a sequence having at least 50%, at least 60%, at least 70%, at least 80%, at least 90%, or more than 90% identity to the sequence set forth in SEQ ID NO:10. In an aspect, a disclosed GJA1 can comprise a mRNA sequence having at least 50%, at least 60%, at least 70%, at least 80%, at least 90%, or more than 90% identity to the sequence set forth in SEQ ID NO:11. In an aspect, a disclosed GJA1 can comprise a genomic sequence having at least 50%, at least 60%, at least 70%, at least 80%, at least 90%, or more than 90% identity to the sequence set forth in SEQ ID NO:12.

In an aspect, a disclosed viral vector or a disclosed non-viral vector can comprise a gRNA targeting one or more cardiac ion channels. In an aspect, a disclosed cardiac ion channel can comprise KCNJ2, KCNH2, GJA1, or any combination thereof. In an aspect, a disclosed viral vector or a disclosed non-viral vector does not comprise a gRNA targeting one or more cardiac ion channels. In an aspect, a disclosed viral vector or a disclosed non-viral vector can exclude comprise a gRNA targeting one or more cardiac ion channels. In an aspect, a disclosed viral vector or a disclosed non-viral vector acid sequencing encoding one or more regulatory elements and/or one or more elements that contribute to and/or confer stabilization of a resulting transcript. In an aspect, a disclosed regulatory element can comprise a promoter, an enhancer, a promoter/enhancer, an internal ribosomal entry site, a transcription terminal signal, a polyadenylation signal, a p2A signal, a woodchuck hepatitis virus post-transcriptional regulatory element (WPRE), a Phi signal-packaging signal (see, e.g., SEQ ID NO:42), a rev responsive element, a 5′-LTR (see, e.g., SEQ ID NO:40), a 3′-LTR (see, e.g., SEQ ID NO:41), an inverted terminal repeat, a nuclear localization signal (NLS), or any combination thereof. In an aspect, a disclosed NLS can comprise the sequence set forth in any one of SEQ ID NO:31-SEQ ID NO:37 or a fragment thereof. In an aspect, a disclosed PolyA sequence can comprise the sequence set forth in SEQ ID NO:38 or SEQ ID NO:39 or a fragment thereof. In an aspect, a disclosed ITR can comprise the sequence set forth in SEQ ID NO:40 or SEQ ID NO:41 or a fragment thereof.

In an aspect, a disclosed viral vector or a disclosed non-viral vector can further a nucleic acid sequence encoding one or more promoters. In an aspect, a disclosed promoter can comprise a SV40 promoter, a U6 promoter, a chicken β-actin promoter, an EF-1α, a CMV promoter, a CMV promoter/enhancer, a fragment thereof, a SFFV promoter, or any combination thereof. In an aspect, a disclosed SFFV promoter can comprise the sequence set forth in SEQ ID NO:43 or SEQ ID NO:44 or a fragment thereof. In an aspect, a disclosed SFFV promoter can comprise a sequence having at least 60%, at least 65%, at least 70%, at least 75%, at least 80%, at least 85%, at least 90%, at least 95%, or greater than 95% identity to the sequence set forth in SEQ ID NO:43 or SEQ ID NO:44 or a fragment thereof. In an aspect, a disclosed U6 promoter can comprise the sequence set forth in SEQ ID NO:48 or a fragment thereof. In an aspect, a disclosed U6 promoter can comprise a sequence having at least 60%, at least 65%, at least 70%, at least 75%, at least 80%, at least 85%, at least 90%, at least 95%, or greater than 95% identity to the sequence set forth in SEQ ID NO:48 or a fragment thereof. In an aspect, a disclosed EF-1α promoter can comprise the sequence set forth in SEQ ID NO:46, SEQ ID NO:47, or a fragment thereof. In an aspect, a disclosed EF-1α promoter can comprise a sequence having at least 60%, at least 65%, at least 70%, at least 75%, at least 80%, at least 85%, at least 90%, at least 95%, or greater than 95% identity to the sequence set forth in SEQ ID NO:46, SEQ ID NO:47, or a fragment thereof. In an aspect, a promoter can be a short EF1α promoter.

In an aspect, a disclosed promoter can be operably linked to the dCas9 endonuclease. In an aspect, a disclosed promoter operably linked to the dCas endonuclease can comprise a disclosed SFFV promoter. In an aspect, a disclosed promoter operably linked to the dCas9 endonuclease can be operably linked to the at least one polypeptide having repressor activity. In an aspect, a disclosed promoter can be operably linked to the dCas9 endonuclease and the at least one polypeptide having effector activity.

In an aspect, a disclosed promoter can be operably linked to a disclosed Zim3-KRAB-dCas9 effector. In an aspect, a disclosed promoter operably can be linked to the at least one guide RNA targeting a gene of interest or portion thereof.

In an aspect, a disclosed promoter operably linked to the at least one guide RNA can comprise a U6 promoter. In an aspect, a disclosed isolated nucleic acid molecule can further comprise a gRNA scaffold.

In an aspect, a disclosed viral vector or a disclosed non-viral vector can further comprise a nucleic acid sequence encoding one or more promoters, wherein a first promoter can be operably linked to the dCas9 endonuclease, and wherein a second promoter can be operably linked to the at least one guide RNA targeting a gene of interest or portion thereof. In an aspect, a disclosed viral vector or a disclosed non-viral vector can further comprise a nucleic acid sequence encoding one or more promoters, wherein a first promoter can be operably linked to the dCas9 endonuclease and the at least one polypeptide having repressor activity, and wherein a second promoter can be operably linked to the at least one guide RNA targeting a gene of interest thereof.

In an aspect of a disclosed vector, a disclosed nucleic acid sequence can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art. In an aspect, any disclosed Cas9 endonuclease, a disclosed polypeptide having enzymatic activities, a disclosed fusion product or a disclosed fusion protein, or any combination thereof can be codon-optimized.

One problem of iPSC-CM that are differentiated using commonly used techniques can be that they are stuck at a relatively immature stage of differentiation with a morphology and marker expression that is very similar to a cardiomyocyte in the early embryonic heart.

In an aspect, a disclosed viral vector or a disclosed non-viral vector can improve or enhancing maturation of iPSC-CMs. In an aspect, a disclosed viral vector or a disclosed non-viral vector can demonstrate an increased expression and/or activity level when compared to a wild-type expression level or control expression level, or when compared to the expression level in the absence of the disclosed nucleic acid molecule. In an aspect, a disclosed viral vector or a disclosed non-viral vector can demonstrate a decreased expression and/or activity level when compared to a wild-type expression level or control expression level, or when compared to the expression level in the absence of the disclosed nucleic acid molecule. In an aspect, a disclosed viral vector or a disclosed non-viral vector can increase the expression and/or activity level of one or more cardiac ion channels when compared to a wild-type expression level or control expression level, or when compared to the expression level in the absence of the disclosed nucleic acid molecule.

In an aspect, a disclosed viral vector or a disclosed non-viral vector can increase the expression level and/or activity level of one or more of KCNJ2, KCNH2, GJA1, or any combination thereof, or when compared to the expression level in the absence of the disclosed nucleic acid molecule. In an aspect, a disclosed viral vector or a disclosed non-viral vector can enhance or improve the functionality of one or more cardiac ion channels when compared to a wild-type expression level or control expression level, or when compared to the expression level in the absence of the disclosed nucleic acid molecule. In an aspect, a disclosed viral vector or a disclosed non-viral vector can enhance or improve the functionality of KCNJ2, KCNH2, GJA1, or any combination thereof, or when compared to the expression level in the absence of the disclosed nucleic acid molecule. In an aspect, a disclosed viral vector or a disclosed non-viral vector can improve or enhance the functionality of one or more cardiac ion channels when compared to a wild-type expression level or control expression level, or when compared to the expression level in the absence of the disclosed nucleic acid molecule. In an aspect, a disclosed viral vector or a disclosed non-viral vector can improve or enhance the functionality of KCNJ2, KCNH2, GJA1, or any combination thereof, or when compared to the expression level in the absence of the disclosed nucleic acid molecule.

In an aspect, a disclosed viral vector or a disclosed non-viral vector can drive or stimulate one or more electrophysiological changes indicative of a mature phenotype in iPSC-CMs when compared to a wild-type expression level or control expression level, or when compared to the expression level in the absence of the disclosed nucleic acid molecule. In an aspect, a disclosed viral vector or a disclosed non-viral vector can drive or stimulate one or more electrophysiological changes (e.g., increase in expression of KCNJ2, KCNH2, GJA1, or any combination thereof) indicative of a mature phenotype in iPSC-CMs, or when compared to the expression level in the absence of the disclosed nucleic acid molecule.

In an aspect, matured iPSC-CMs can be characterized by mitochondrial maturation, increased oxidative capacity and enhanced fatty acid use for energy production. For example, the structural, electrophysiological, contractile, and metabolic characteristics of iPSC-CMs can be under-developed when compared to adulty cardiomyocytes. In an aspect, a disclosed viral vector or a disclosed non-viral vector can be used to generate a mature structural, electrophysiological, contractile, and metabolic profile.

In an aspect, maturation of iPSC-CMs improved or enhanced by a disclosed viral vector or a disclosed non-viral vector can be assessed by an increase in the expression level of PDK4, CD36, PPARA, ATP5, LPL, SCD, PPARD, ACADVL, ACAT1, DGAT1, PPARGC1A, ESRRA, N2B, CAV3, SERCA2, CPT1A/1B, or any combination thereof. In an aspect, maturation of iPSC-CMs improved or enhanced by a disclosed viral vector or a disclosed non-viral vector can be assessed by a decrease in the expression level of ALDOA, HK1, HK2, PGK1, GAPDH, LDHA, or any combination thereof.

In an aspect, maturation of iPSC-CMs improved or enhanced by a disclosed viral vector or a disclosed non-viral vector can be assessed by an increase in mitochondrial numbers and size, an increase in the number of peri-sarcomeric mitochondria, a decrease in the number of perinuclear mitochondria, or any combination thereof.

In an aspect, maturation of iPSC-CMs improved or enhanced by a disclosed viral vector or non-viral vector can be assessed by an increase in the expression of genes encoding electronic transport chain (ETC) proteins. In an aspect, maturation of iPSC-CMs improved or enhanced by a disclosed viral vector or a disclosed non-viral vector can be assessed by a reduction in glucose uptake, glycogen storage, lactate production, hexokinase activity, the proportion of glycolysis-related ATP production, or any combination thereof.

In an aspect, maturation of iPSC-CMs improved or enhanced by a disclosed viral vector or a disclosed non-viral vector can be assessed by an increase in the relative contribution of fatty acid oxidation (FAO) to ATP production. In an aspect, a disclosed viral vector or a disclosed non-viral vector can be used to enhance or increase or promoter the metabolic maturation of iPSC-CMs (e.g., improved mitochondrial structure and function, decreased glycolytic activity, increased FAO, increased ATP production, or any combination thereof).

In an aspect, a disclosed viral vector or a disclosed non-viral vector can be used to enhance or increase or promote maturation of iPSC-CMs, which can be used to investigate cardiomyopathy disease mechanisms or can be used to interrogate cardiac disease modeling or can be used to screen drugs for efficacy and/or safety.

In an aspect, a disclosed viral vector or a disclosed non-viral vector can be used to enhance or increase or promote maturation of iPSC-CMs as measured and/or assessed by contractile force, morphology, electrophysiology, calcium handling characteristics, metabolic profile, or any combination thereof.

In an aspect, maturation of iPSC-CMs enhanced or increased or promoted by a disclosed viral vector or a disclosed non-viral vector can be assessed by beating when stimulated with a force closer to around 40-80 mN/mm² (e.g., indicative of mature CMs) rather than a force closer to around 0.08-4 mN/mm² (e.g., indicative of immature hiPSC-CMs). In an aspect, maturation of iPSC-CMs enhanced or increased or promoted by a disclosed viral vector or a disclosed non-viral vector can be assessed by a conduction velocity around 60 cm/s (e.g., indicative of mature CMs) rather than a conduction velocity closer to around 10-20 cm/s (e.g., indicative of immature hiPSC-CMs). In an aspect, maturation of iPSC-CMs enhanced or increased or promoted by a disclosed viral vector or a disclosed non-viral vector can be assessed by beating when stimulated with an upstroke velocity about 150-350 V/s (e.g., indicative of mature CMs) rather than an upstroke velocity closer to around 10-50 V/s (e.g., indicative of immature hiPSC-CMs).

In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed viral vector can be an adenoviral vector. In an aspect, a disclosed viral vector can be a lentiviral vector. Viral vectors are known to the art.

In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV218, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.cB, AAV-PHP.S, or AAV-F. In an aspect, a disclosed AAV vector can be AAV-Rh74 or a related variant (e.g., capsid variants like RHM4-1).

In an aspect, a disclosed viral vector or non-viral vector can be administered to a subject in need thereof. In an aspect, a disclosed subject can have one or more cardiac diseases or disorders. In an aspect, a disclosed cardiac disease or disorder can comprise myocardial infarction or ischemic cardiomyopathy. In an aspect, a disclosed cardiac disease or disorder can comprise heart failure. In an aspect, a disclosed cardiac disease or disorder can comprise coronary artery disease or an arrhythmia. Cardiac diseases and disorders are known to the skilled person in the art. In an aspect, a disclosed viral vector or non-viral vector can be added to an implantable cardiac patch. Cardiac patches are known to the art. In an aspect, a disclosed implantable cardiac patch can be provided to a subject in need thereof.

In an aspect, a disclosed viral vector or non-viral vector can slow or prevent progression of a cardiac disease or disorder in a subject in need thereof. In an aspect, slowing or preventing disease progression or a cardiac disease or disorder can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cardiac disease or disorder; (vii) reversing, inhibiting, preventing, stabilizing, and/or slowing the rate of progression of a cardiac disease or disorder, or (viii) any combination thereof.

In an aspect, a disclosed viral vector or disclosed non-viral vector can decrease or minimize the intensity and/or duration of an arrhythmic event or the number or frequency of arrhythmic events.

In an aspect, a disclosed viral vector or disclosed non-viral vector can be used to transform iPSC-CMs into matured CMs or more matured CMs when compared to the CMs prior to contact with the disclosed viral vector or disclosed non-viral vector. In an aspect, matured CMs can be used in several applications. For example, CMs matured by a disclosed viral vector or disclosed non-viral vector or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, a disclosed viral vector or disclosed non-viral vector can be used to upregulate the expression of ion channels (e.g., KCNJ2, KCNH2, GJA1, etc.) indicative of adult CMs for use in drug evaluation (e.g., efficacy, toxicity, safety, etc.). In an aspect, the upregulation of ion channel expression in matured CMs can be a useful tool in the development of relevant cardiac therapies. In an aspect, matured CMs can be helpful in ensuring that a drug is non-toxic and does not have a proarrhythmic effects prior to administration to a subject in need thereof. In an aspect, matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stercolithography, inkjet, etc.). In an aspect, matured CMs can be used in a disclosed therapeutic application, for example, an application to treat the disease progression of a subject having a diseased or damaged heart. In an aspect, a disclosed viral vector or disclosed non-viral vector can be used stabilize repolarization, upregulate conduction velocity, stabilize the resting membrane potential, reduce spontaneous firing, or any combination thereof. In an aspect, matured CMs can demonstrate stabilized repolarization, upregulated conduction velocity, stabilized the resting membrane potential, reduced spontaneous firing, or any combination thereof.

### 3. Plasmids

Disclosed herein is a plasmid comprising a disclosed nucleic acid molecule. Disclosed herein is a plasmid comprising the sequence set forth in SEQ ID NO:63 (pHR-UCOE-SFFV-dCas9-mCherry-Zim3-KRAB), SEQ ID NO:64 (pAV[Exp]-mCherry-SFFV-dCas9/KRAB/McCP2), or SEQ ID NO:65 (p154473-attachment_w5DDpndhwpbDmnEJw54).

Disclosed herein is a plasmid comprising a nucleic acid sequence encoding (i) at least one zinc finger protein, (ii) at least one polypeptide having effector activity, and (iii) a deactivated Cas9 (dCas9) endonuclease. Disclosed herein is a plasmid comprising a nucleic acid sequence encoding a Zim3-KRAB-dCas9 effector. Disclosed herein is a plasmid comprising a nucleic acid sequence encoding a Zim3-KRAB-MeCP2-dCas9 effector.

In an aspect of a disclosed plasmid, a disclosed encoded zinc finger protein can comprise the sequence set forth in SEQ ID NO:01 or a fragment thereof. In an aspect of a disclosed plasmid, a disclosed zinc finger protein can comprise the mRNA sequence set forth in SEQ ID NO:02 or a fragment thereof. In an aspect of a disclosed plasmid, a disclosed zinc finger protein can comprise the genomic sequence set forth in SEQ ID NO:03 or a fragment thereof.

In an aspect of a disclosed plasmid, a disclosed polypeptide having effector activity can comprise the sequence set forth in SEQ ID NO:23 or a fragment thereof. In an aspect of a disclosed plasmid, a disclosed encoded polypeptide having effector activity can comprise the sequence set forth in SEQ ID NO:24 or SEQ ID NO:25 or a fragment thereof. In an aspect of a disclosed plasmid, a disclosed polypeptide having effector activity can comprise the sequence set forth in SEQ ID NO:26 or a fragment thereof.

In an aspect of a disclosed plasmid, a disclosed deactivated Cas (dCas9) endonuclease can comprise the sequence set forth in any one of SEQ ID NO:13-SEQ ID NO:19 or a fragment thereof. In an aspect of a disclosed plasmid, a disclosed encoded deactivated Cas (dCas9) endonuclease can comprise the sequence set forth in any one of SEQ ID NO:20-SEQ ID NO:22 or a fragment thereof.

In an aspect of a disclosed plasmid, a disclosed effector activity can comprise transcription activation activity or transcription repression activity. In an aspect of a disclosed plasmid, a disclosed effector activity can be paradoxical. For example, if expected to repress transcription, then in a paradoxical aspect, a disclosed effector can activate transcription. If expected to activate transcription, then in a paradoxical aspect, a disclosed effector can repress transcription. In an aspect, a disclosed plasmid can be purified and/or isolated.

In an aspect of a disclosed plasmid, a disclosed effector can demonstrate an increased expression and/or activity level when compared to wild-type or control expression level. In an aspect of a disclosed plasmid, a disclosed effector can demonstrate a decreased expression and/or activity level when compared to wild-type or control expression level.

In an aspect, a disclosed zinc finger protein can comprise Zinc Finger Imprinted 3 (Zim3). IN an aspect, Zim3 can also be called by or known as ZFP654 and ZFP264. In an aspect, a disclosed zinc finger protein can comprise Zim3/ZNF657/ZNF264.

In an aspect, Zim3 can be described by HGNC Ref. No. 16366, NCBI Gene No. 114026, Ensembl Ref. No. ENSG00000141946, UniProtKB/Swiss-Prot Ref. No. Q96PE6, or any combination thereof.

In an aspect, Zim3 (Zinc Finger Imprinted 3) is a Protein Coding gene. In an aspect, Zim3 can enable DNA-binding transcription factor activity, RNA polymerase II-specific and RNA polymerase II cis-regulatory region sequence-specific DNA binding activity, can regulate transcription by RNA polymerase II, can be active in nucleus, or any combination thereof.

In an aspect, a disclosed encoded Zim3 can comprise the sequence set forth in SEQ ID NO:01 or a fragment thereof. In an aspect, a disclosed Zim3 can comprise the mRNA sequence set forth in SEQ ID NO:02 or a fragment thereof. In an aspect, a disclosed Zim3 can comprise the genomic sequence set forth in SEQ ID NO:03 or a fragment thereof.

In an aspect, a disclosed encoded Zim3 can comprise a sequence having at least 50%, at least 60%, at least 70%, at least 80%, at least 90%, or more than 90% identity to the sequence set forth in SEQ ID NO:01. In an aspect, a disclosed Zim3 can comprise a mRNA sequence having at least 50%, at least 60%, at least 70%, at least 80%, at least 90%, or more than 90% identity to the sequence set forth in SEQ ID NO:02. In an aspect, a disclosed Zim3 can comprise a genomic sequence having at least 50%, at least 60%, at least 70%, at least 80%, at least 90%, or more than 90% identity to the sequence set forth in SEQ ID NO:03.

In an aspect, a disclosed dCas endonuclease can comprise a dCas9 endonuclease. In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER.

In an aspect, a disclosed dCas9 can comprise a catalytically dead mutant of the Cas9 endonuclease from the Streptococcus pyogenes Type II CRISPR/Cas system. In an aspect, a disclosed dCas9 can comprise a catalytically dead mutant of the Cas9 endonuclease from the Staphylococcus aureus Type II CRISPR/Cas system. In an aspect, a disclosed dCas9 can comprise a catalytically dead mutant of the Cas9 endonuclease from the Campylobacter jejuni Type II CRISPR/Cas system.

In an aspect, a disclosed dSpCas9 can comprise the sequence set forth in SEQ ID NO:13 or SEQ ID NO:14 or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:13 or SEQ ID NO:14. In an aspect, a disclosed encoded dSpCas9 can comprise the sequence set forth in SEQ ID NO:20 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect, a disclosed dSaCas9 can comprise the sequence set forth in SEQ ID NO:15 or SEQ ID NO:16 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:15 or SEQ ID NO:16 or a fragment thereof. In an aspect, a disclosed encoded dSaCas9 can comprise the sequence set forth in SEQ ID NO:21 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dCjCas9 can comprise the sequence set forth in SEQ ID NO:17 or SEQ ID NO:18 or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:17 or SEQ ID NO:18 or a fragment thereof. In an aspect, a disclosed encoded dCjCas9 can comprise the sequence set forth in SEQ ID NO:22 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:22 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:19.

In an aspect, a disclosed encoded polypeptide having effector activity can comprise transcription activation activity or transcription repression activity. In an aspect, a disclosed effector activity can comprise transcription activation activity or transcription repression activity. In an aspect, a disclosed effector activity can be paradoxical. For example, if expected to repress transcription, then in a paradoxical aspect, a disclosed effector can activate transcription. If expected to activate transcription, then in a paradoxical aspect, a disclosed effector can repress transcription.

In an aspect, a disclosed polypeptide having effector activity can comprise KRAB or KRAB-MeCP2. In an aspect, KRAB refers to Krüppel-Associated Box. In an aspect, KRAB-MeCP2 refers to Krüppel-Associated Box and Methyl-CpG Binding Protein 2 (MeCP2). In an aspect, a disclosed KRAB can comprise the sequence set forth in SEQ ID NO:23 or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:23 or a fragment thereof. In an aspect, a disclosed encoded KRAB can comprise the sequence set forth in SEQ ID NO:24 or SEQ ID NO:25 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:24 or SEQ ID NO:25 or a fragment thereof. In an aspect, a disclosed KRAB-MeCP2 can comprise the sequence set forth in SEQ ID NO:26 or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:26 or a fragment thereof. In an aspect, a disclosed encoded KRAB-MeCP2 can comprise the sequence set forth in SEQ ID NO:27 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:27 or a fragment thereof.

In an aspect, a disclosed encoded dCas9 endonuclease is fused to the at least one encoded polypeptide having effector activity (e.g., KRAB or KRAB-MeCP2). In an aspect, a disclosed dCas9 endonuclease can comprise dSpCas9 and a disclosed encoded polypeptide can comprise KRAB or KRAB-MeCP2. In an aspect, a disclosed dCas9 endonuclease can comprise dSaCas9 and a disclosed encoded polypeptide can comprise KRAB or KRAB-MeCP2. In an aspect, a disclosed dCas9 endonuclease can comprise dCjCas9 and a disclosed polypeptide can comprise KRAB or KRAB-MeCP2.

In an aspect, a disclosed dCas9 endonuclease can comprise dVQR, dEQR, or dVRER and a disclosed encoded polypeptide can comprise KRAB or KRAB-MeCP2. In an aspect, a disclosed dCas9 endonuclease can comprise dSpCas9, dSaCas9, or dCjCas9 and a disclosed encoded polypeptide can comprise KRAB or KRAB-MeCP2. In an aspect, a disclosed dCas9 endonuclease can comprise dSpCas9, dSaCas9, or dCjCas9, and KRAB or KRAB-MeCP2. In an aspect, a disclosed dCas9 and a disclosed KRAB-MeCP2 can comprise the sequence set forth in SEQ ID NO:28.

In an aspect, a disclosed encoded Zim3, a disclosed encoded polypeptide having effector activity, and a disclosed dCas9 endonuclease can be joined. In an aspect, a disclosed encoded Zim3, a disclosed encoded KRAB or KRAB-McCP2, and a disclosed dCas9 endonuclease can be joined. In an aspect, a disclosed encoded Zim3, a disclosed encoded KRAB or KRAB-McCP2, and a disclosed dSpCas9 endonuclease can be joined. In an aspect, a disclosed encoded Zim3, a disclosed encoded KRAB or KRAB-McCP2, and a disclosed dSaCas9 endonuclease can be joined. In an aspect, a disclosed encoded Zim3, a disclosed encoded KRAB or KRAB-McCP2, and a disclosed dCjCas9 endonuclease can be joined. In an aspect, a disclosed dCas9 endonuclease can comprise dVQR, dEQR, or dVRER and a disclosed encoded polypeptide can comprise KRAB or KRAB-MeCP2. In an aspect, a disclosed dCas9 endonuclease can comprise dSpCas9, dSaCas9, or dCjCas9 and a disclosed encoded polypeptide can comprise KRAB or KRAB-McCP2. In an aspect, a disclosed zinc finger protein can comprise Zim3. In an aspect, a disclosed nucleic acid sequence can comprise Zim3-KRAB-dCas9. In an aspect, a disclosed nucleic acid sequence can comprise Zim3-KRAB-MeCP2-dCas9. In an aspect, a disclosed nucleic acid sequence can encode Zim3-KRAB-dCas9. In an aspect, a disclosed nucleic acid sequence can encode Zim3-KRAB-MeCP2-dCas9. In an aspect, Zim3-KRAB-dCas9 can effect transcription activation activity. In an aspect, Zim3-KRAB-dCas9 can effect transcription repression activity. In an aspect, Zim3-KRAB-dCas9 can effect transcription activation activity, which can be paradoxical.

In an aspect, a disclosed plasmid can encode the sequence set forth in any one of SEQ ID NO:24, SEQ ID NO:25, SEQ ID NO:27, or SEQ ID NO:28, and can encode the sequence set forth in any one of SEQ ID NO:20-SEQ ID NO:22. In an aspect, a disclosed plasmid can comprise the sequence set forth in SEQ ID NO:23 and the sequence set forth in SEQ ID NO:14.

In an aspect, a disclosed plasmid can upregulate the expression of one or more ion channels. In an aspect, a disclosed plasmid can upregulate the expression of one or more cardiac ion channels. In an aspect, a disclosed cardiac ion channel can comprise KCNJ2, KCNH2, GJA1, or any combination thereof. In an aspect, a disclosed plasmid can upregulate the expression of one or more KCNJ2, KCNH2, GJA1, or any combination thereof. In an aspect, a disclosed ion channel can be Potassium Inwardly Rectifying Channel Subfamily J Member 2 (KCNJ2). In an aspect, KCNJ2 can be described by HGNC Ref. No. 6263, NCBI Gene No. 3759, Ensembl Ref. No. ENSG00000123700, OMIM Ref. No. 600681, UniProtKB/Swiss-Prot Ref. No. P63252, or any combination thereof. In an aspect, KCNJ2 can be associated with Andersen Cardiodysrhythmic Periodic Paralysis and Short Qt Syndrome 3. In an aspect, a disclosed encoded KCNJ2 can comprise the sequence set forth in SEQ ID NO:04 or a fragment thereof. In an aspect, a disclosed KCNJ2 can comprise the mRNA sequence set forth in SEQ ID NO:05 or a fragment thereof. In an aspect, a disclosed KCNJ2 can comprise the genomic sequence set forth in SEQ ID NO:06 or a fragment thereof. In an aspect, a disclosed encoded KCNJ2 can comprise a sequence having at least 50%, at least 60%, at least 70%, at least 80%, at least 90%, or more than 90% identity to the sequence set forth in SEQ ID NO:04. In an aspect, a disclosed KCNJ2 can comprise a mRNA sequence having at least 50%, at least 60%, at least 70%, at least 80%, at least 90%, or more than 90% identity to the sequence set forth in SEQ ID NO:05. In an aspect, a disclosed KCNJ2 can comprise a genomic sequence having at least 50%, at least 60%, at least 70%, at least 80%, at least 90%, or more than 90% identity to the sequence set forth in SEQ ID NO:06.

In an aspect, a disclosed ion channel can be potassium voltage-gated channel subfamily H member 2 (KCNH2). In an aspect, KCNH2 can be described by HGNC Ref. No. 6251, NCBI Gene No. 3757, Ensembl Ref. No. ENSG00000055118, OMIM Ref. No. 152427, UniProtKB/Swiss-Prot Ref. No. Q12809, or any combination thereof. In an aspect, KCNH2 can refer to a voltage-activated potassium channel found in cardiac muscle, nerve cells, and microglia. In an aspect, 4 copies of this protein interact with one copy of the KCNE2 protein to form a functional potassium channel. In an aspect, a disclosed mutation in this gene encoding KCNH2 can cause long QT syndrome type 2 (LQT2) and Short Qt Syndrome 1. In an aspect, a disclosed encoded KCNH2 can comprise the sequence set forth in SEQ ID NO:07 or a fragment thereof.

In an aspect, a disclosed KCNH2 can comprise the mRNA sequence set forth in SEQ ID NO:08 or a fragment thereof. In an aspect, a disclosed KCNH2 can comprise the genomic sequence set forth in SEQ ID NO:09 or a fragment thereof. In an aspect, a disclosed encoded KCNH2 can comprise a sequence having at least 50%, at least 60%, at least 70%, at least 80%, at least 90%, or more than 90% identity to the sequence set forth in SEQ ID NO:07. In an aspect, a disclosed KCNH2 can comprise a mRNA sequence having at least 50%, at least 60%, at least 70%, at least 80%, at least 90%, or more than 90% identity to the sequence set forth in SEQ ID NO:08. In an aspect, a disclosed KCNH2 can comprise a genomic sequence having at least 50%, at least 60%, at least 70%, at least 80%, at least 90%, or more than 90% identity to the sequence set forth in SEQ ID NO:09.

In an aspect, a disclosed ion channel can be gap junction protein alpha 1 (GJA1). In an aspect, GJA1 can be described by HGNC Ref. No. 4274, NCBI Gene No. 2697, Ensembl Ref. No. ENSG00000152661, OMIM Ref. No. 121014, UniProtKB/Swiss-Prot Ref. No. P17302, or any combination thereof. In an aspect, GJA1 can be associated with gap junctions, which are composed of arrays of intercellular channels that provide a route for the diffusion of low molecular weight materials from cell to cell. In an aspect, the encoded protein GJA1 can be the major protein of gap junctions in the heart, having a preimminent role in the synchronized contraction of the heart and in embryonic development. In an aspect, GJA1 can be associated with Oculodentodigital Dysplasia and Syndactyly, Type Iii. In an aspect, a disclosed encoded GJA1 can comprise the sequence set forth in SEQ ID NO:10 or a fragment thereof. In an aspect, a disclosed GJA1 can comprise the mRNA sequence set forth in SEQ ID NO:11 or a fragment thereof. In an aspect, a disclosed GJA1 can comprise the genomic sequence set forth in SEQ ID NO:12 or a fragment thereof. In an aspect, a disclosed encoded GJA1 can comprise a sequence having at least 50%, at least 60%, at least 70%, at least 80%, at least 90%, or more than 90% identity to the sequence set forth in SEQ ID NO:10. In an aspect, a disclosed GJA1 can comprise a mRNA sequence having at least 50%, at least 60%, at least 70%, at least 80%, at least 90%, or more than 90% identity to the sequence set forth in SEQ ID NO:11. In an aspect, a disclosed GJA1 can comprise a genomic sequence having at least 50%, at least 60%, at least 70%, at least 80%, at least 90%, or more than 90% identity to the sequence set forth in SEQ ID NO:12.

In an aspect, a disclosed plasmid can comprise a gRNA targeting one or more cardiac ion channels. In an aspect, a disclosed plasmid does not comprise a gRNA targeting one or more cardiac ion channels. In an aspect, a disclosed plasmid can exclude comprise a gRNA targeting one or more cardiac ion channels.

In an aspect, a disclosed plasmid can encode one or more regulatory elements and/or one or more elements that contribute to and/or confer stabilization of a resulting transcript. In an aspect, a disclosed regulatory element can comprise a promoter, an enhancer, a promoter/enhancer, an internal ribosomal entry site, a transcription terminal signal, a polyadenylation signal, a p2A signal, a woodchuck hepatitis virus post-transcriptional regulatory element (WPRE), a Phi signal-packaging signal (see, e.g., SEQ ID NO:42), a rev responsive element, a 5′-LTR (see, e.g., SEQ ID NO:40), a 3′-LTR (see, e.g., SEQ ID NO:41), an inverted terminal repeat, a nuclear localization signal (NLS), or any combination thereof.

In an aspect, a disclosed NLS can comprise the sequence set forth in any one of SEQ ID NO:31-SEQ ID NO:37 or a fragment thereof. In an aspect, a disclosed PolyA sequence can comprise the sequence set forth in SEQ ID NO:38 or SEQ ID NO:39 or a fragment thereof. In an aspect, a disclosed ITR can comprise the sequence set forth in SEQ ID NO:40 or SEQ ID NO:41 or a fragment thereof.

In an aspect, a disclosed plasmid can further a nucleic acid sequence encoding one or more promoters. In an aspect, a disclosed promoter can comprise a SV40 promoter, a U6 promoter, a chicken β-actin promoter, an EF-1α, a CMV promoter, a CMV promoter/enhancer, a fragment thereof, a SFFV promoter, or any combination thereof. In an aspect, a disclosed SFFV promoter can comprise the sequence set forth in SEQ ID NO:43 or SEQ ID NO:44 or a fragment thereof. In an aspect, a disclosed SFFV promoter can comprise a sequence having at least 60%, at least 65%, at least 70%, at least 75%, at least 80%, at least 85%, at least 90%, at least 95%, or greater than 95% identity to the sequence set forth in SEQ ID NO:43 or SEQ ID NO:44 or a fragment thereof.

In an aspect, a disclosed U6 promoter can comprise the sequence set forth in SEQ ID NO:48 or a fragment thereof. In an aspect, a disclosed U6 promoter can comprise a sequence having at least 60%, at least 65%, at least 70%, at least 75%, at least 80%, at least 85%, at least 90%, at least 95%, or greater than 95% identity to the sequence set forth in SEQ ID NO:48 or a fragment thereof. In an aspect, a disclosed EF-1α promoter can comprise the sequence set forth in SEQ ID NO:46, SEQ ID NO:47, or a fragment thereof. In an aspect, a disclosed EF-1α promoter can comprise a sequence having at least 60%, at least 65%, at least 70%, at least 75%, at least 80%, at least 85%, at least 90%, at least 95%, or greater than 95% identity to the sequence set forth in SEQ ID NO:46, SEQ ID NO:47, or a fragment thereof. In an aspect, a promoter can be a short EFla promoter.

In an aspect, a disclosed promoter can be operably linked to the dCas9 endonuclease. In an aspect, a disclosed promoter operably linked to the dCas9 endonuclease can comprise a disclosed SFFV promoter. In an aspect, a disclosed promoter operably linked to the dCas9 endonuclease can be operably linked to the at least one polypeptide having repressor activity. In an aspect, a disclosed promoter can be operably linked to the dCas9 endonuclease and the at least one polypeptide having effector activity. In an aspect, a disclosed promoter can be operably linked to a disclosed Zim3-KRAB-dCas9 effector.

In an aspect, a disclosed promoter operably can be linked to the at least one guide RNA targeting a gene of interest or portion thereof. In an aspect, a disclosed promoter operably linked to the at least one guide RNA can comprise a U6 promoter.

In an aspect, a disclosed isolated nucleic acid molecule can further comprise a gRNA scaffold.

In an aspect, a disclosed plasmid can further comprise a nucleic acid sequence encoding one or more promoters, wherein a first promoter can be operably linked to the dCas9 endonuclease, and wherein a second promoter can be operably linked to the at least one guide RNA targeting a gene of interest or portion thereof. In an aspect, a disclosed plasmid can further comprise a nucleic acid sequence encoding one or more promoters, wherein a first promoter can be operably linked to the dCas9 endonuclease and the at least one polypeptide having an repressor activity, and wherein a second promoter can be operably linked to the at least one guide RNA targeting a gene of interest thereof.

In an aspect, a disclosed plasmid can comprise a nucleic acid sequence that can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art. In an aspect, any disclosed Cas9 endonuclease, a disclosed polypeptide having enzymatic activities, a disclosed fusion product or a disclosed fusion protein, or any combination thereof can be codon-optimized.

In an aspect, a disclosed plasmid can be administered to a subject in need thereof. In an aspect, a disclosed subject can have one or more cardiac diseases or disorders.

In an aspect, a disclosed cardiac disease or disorder can comprise myocardial infarction or ischemic cardiomyopathy. In an aspect, a disclosed cardiac disease or disorder can comprise heart failure. In an aspect, a disclosed cardiac disease or disorder can comprise coronary artery disease or an arrhythmia. Cardiac diseases and disorders are known to the skilled person in the art. In an aspect, a disclosed plasmid can be added to an implantable cardiac patch. Cardiac patches are known to the art. In an aspect, a disclosed implantable cardiac patch can be provided to a subject in need thereof.

In an aspect, a disclosed plasmid can slow or prevent progression of a cardiac disease or disorder in a subject in need thereof. In an aspect, slowing or preventing disease progression or a cardiac disease or disorder can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cardiac disease or disorder; (vii) reversing, inhibiting, preventing, stabilizing, and/or slowing the rate of progression of a cardiac disease or disorder, or (viii) any combination thereof.

In an aspect, a disclosed plasmid can decrease or minimize the intensity and/or duration of an arrhythmic event or the number or frequency of arrhythmic events.

In an aspect, a disclosed plasmid can be used to transform iPSc-CMs into matured CMs or more matured CMs when compared to the CMs prior to contact with the disclosed viral vector or disclosed non-viral vector. In an aspect, matured CMs can be used in several applications. For example, CMs matured by a disclosed plasmid or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, a disclosed plasmid can be used to upregulate the expression of ion channels (e.g., KCNJ2, KCNH2, GJA1, etc.) indicative of adult CMs for use in drug evaluation (e.g., efficacy, toxicity, safety, etc.). In an aspect, the upregulation of ion channel expression in matured CMs can be a useful tool in the development of relevant cardiac therapies. In an aspect, matured CMs can be helpful in ensuring that a drug is non-toxic and does not have a proarrhythmic effects prior to administration to a subject in need thereof. In an aspect, matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stereolithography, inkjet, etc.). In an aspect, matured CMs can be used in a disclosed therapeutic application, for example, an application to treat the disease progression of a subject having a diseased or damaged heart. In an aspect, a disclosed plasmid can be used stabilize repolarization, upregulate conduction velocity, stabilize the resting membrane potential, reduce spontaneous firing, or any combination thereof. In an aspect, matured CMs can demonstrate stabilized repolarization, upregulated conduction velocity, stabilized the resting membrane potential, reduced spontaneous firing, or any combination thereof.

4. iPSC-CMs

Disclosed herein is one or more iPSC-CMs contacted with a disclosed nucleic acid molecule, a disclosed viral vector, a disclosed non-viral vector, a disclosed plasmid, a disclosed pharmaceutical formulation, or any combination. Disclosed herein is one or more iPSC-CMs transfected with a disclosed nucleic acid molecule, a disclosed non-viral vector, a disclosed plasmid, a pharmaceutical formulation, or any combination. Disclosed herein is one or more iPSC-CMs transduced with a disclosed viral vector or a pharmaceutical formulation. Disclosed herein is an iPSC-CM transduced by a disclosed nucleic acid molecule. Disclosed herein is an iPSC-CM transduced by a nucleic acid molecule comprising a nucleic acid sequence encoding (i) at least one zinc finger protein, (ii) at least one polypeptide having effector activity, and (iii) a deactivated Cas9 (dCas9) endonuclease. Disclosed herein is an iPSC-CM transduced by a nucleic acid molecule comprising a sequence encoding a Zim3-KRAB-dCas9 effector. Disclosed herein is an iPSC-CM transduced by a nucleic acid molecule comprising a sequence encoding a Zim3-KRAB-MeCP2-dCas9 effector.

Disclosed herein is an iPSC-CM transfected by a disclosed viral vector. Disclosed herein is an iPSC-CM transfected by a viral vector comprising a nucleic acid sequence encoding (i) at least one zinc finger protein, (ii) at least one polypeptide having effector activity, and (iii) a deactivated Cas9 (dCas9) endonuclease. Disclosed herein is an iPSC-CM transfected by a viral vector comprising a nucleic acid sequence encoding a Zim3-KRAB-dCas9 effector. Disclosed herein is an iPSC-CM transfected by a viral vector comprising a nucleic acid sequence encoding a Zim3-KRAB-MeCP2-dCas9 effector. Disclosed herein is an iPSC-CM transfected by a recombinant viral vector comprising a disclosed nucleic acid molecule. Disclosed herein is an iPSC-CM transfected by a recombinant viral vector comprising a nucleic acid sequence encoding (i) at least one zinc finger protein, (ii) at least one polypeptide having effector activity, and (iii) a deactivated Cas9 (dCas9) endonuclease. Disclosed herein is an iPSC-CM transfected by a recombinant viral vector comprising a nucleic acid sequence encoding a Zim3-KRAB-dCas9 effector. Disclosed herein is an iPSC-CM transfected by a recombinant viral vector comprising a nucleic acid sequence encoding a Zim3-KRAB-MeCP2-dCas9 effector.

Disclosed herein is an iPSC-CM transduced by a disclosed non-viral vector. Disclosed herein is an iPSC-CM transduced by a non-viral vector comprising a nucleic acid sequence encoding (i) at least one zinc finger protein, (ii) at least one polypeptide having effector activity, and (iii) a deactivated Cas9 (dCas9) endonuclease. Disclosed herein is an iPSC-CM transduced by a non-viral vector comprising a nucleic acid sequence encoding a Zim3-KRAB-dCas9 effector. Disclosed herein is an iPSC-CM transduced by a non-viral vector comprising a nucleic acid sequence encoding a Zim3-KRAB-MeCP2-dCas9 effector. Disclosed herein is an iPSC-CM transduced by a recombinant non-viral vector comprising a disclosed nucleic acid molecule. Disclosed herein is an iPSC-CM transduced by a recombinant non-viral vector comprising a nucleic acid sequence encoding (i) at least one zinc finger protein, (ii) at least one polypeptide having effector activity, and (iii) a deactivated Cas9 (dCas9) endonuclease. Disclosed herein is an iPSC-CM transduced by a recombinant non-viral vector comprising a nucleic acid sequence encoding a Zim3-KRAB-dCas9 effector. Disclosed herein is an iPSC-CM transduced by a recombinant non-viral vector comprising a nucleic acid sequence encoding a Zim3-KRAB-MeCP2-dCas9 effector.

Disclosed herein is an iPSC-CM transfected by a disclosed plasmid. Disclosed herein is an iPSC-CM transfected by a disclosed plasmid comprising a disclosed nucleic acid molecule. Disclosed herein is an iPSC-CM transfected by a disclosed plasmid comprising the sequence set forth in SEQ ID NO:63 (pHR-UCOE-SFFV-dCas9-mCherry-Zim3-KRAB) or SEQ ID NO:64 (pAV[Exp]-mCherry-SFFV-dCas9/KRAB/MeCP2).

Disclosed herein is an iPSC-CM transfected by a plasmid comprising a nucleic acid sequence encoding (i) at least one zinc finger protein, (ii) at least one polypeptide having effector activity, and (iii) a deactivated Cas9 (dCas9) endonuclease. Disclosed herein is an iPSC-CM transfected by a plasmid comprising a nucleic acid sequence encoding a Zim3-KRAB-dCas9 effector. Disclosed herein is an iPSC-CM transfected by a plasmid comprising a nucleic acid sequence encoding a Zim3-KRAB-MeCP2-dCas9 effector.

In an aspect, following the contacting of the iPSC-CMs with a disclosed nucleic acid molecule, a disclosed viral vector, a disclosed non-viral vector, a disclosed plasmid, a disclosed pharmaceutical formulation, or any combination thereof, the iPSC-CMs can demonstrate improved or enhanced maturation. In an aspect, following either (i) the transfection of the iPSC-CMs with a disclosed nucleic acid molecule, a disclosed non-viral vector, a disclosed plasmid, a pharmaceutical formulation, or any combination, or (ii) the transduction of the iPSC-CMs with a disclosed viral vector or a pharmaceutical formulation, the iPSC-CMs can demonstrate improved or enhanced maturation.

In an aspect, following a disclosed contacting or a disclosed transfection or a disclosed transduction of the iPSC-CMs, the functionality of one or more cardiac ion channels can be improved or enhanced when compared to a wild-type expression level or control expression level, or when compared to the expression level in the absence of the contacting or the transfection or transduction.

In an aspect, following a disclosed contacting or a disclosed transfection or a disclosed transduction of the iPSC-CMs, the functionality of KCNJ2, KCNH2, GJA1, or any combination thereof can be improved or enhanced when compared to the expression level in the absence of the contacting or the transfection or transduction.

In an aspect, a disclosed contacting or a disclosed transfection or a disclosed transduction of the iPSC-CMs can drive or stimulate one or more electrophysiological changes indicative of a mature phenotype in iPSC-CMs when compared to a wild-type expression level or control expression level, or when compared to the expression level in the absence of the contacting or the transfection or transduction.

In an aspect, a disclosed contacting or a disclosed transfection or a disclosed transduction of the iPSC-CMs can drive or stimulate one or more electrophysiological changes (e.g., increase in expression of KCNJ2, KCNH2, GJA1, or any combination thereof) indicative of a mature phenotype in the iPSC-CMs, or when compared to the expression level in the absence of the contacting or the transfection or transduction.

In an aspect, matured iPSC-CMs can be characterized by mitochondrial maturation, increased oxidative capacity, and enhanced fatty acid use for energy production. For example, the structural, electrophysiological, contractile, and metabolic characteristics of the iPSC-CMs can be under-developed when compared to adult cardiomyocytes. In an aspect, a disclosed contacting or a disclosed transfection or a disclosed transduction of the iPSC-CMs can be used to generate a mature structural, electrophysiological, contractile, and metabolic profile.

In an aspect, maturation of iPSC-CMs improved or enhanced by a disclosed contacting or a disclosed transfection or a disclosed transduction can be assessed by an increase in the expression level of PDK4, CD36, PPARA, ATP5, LPL, SCD, PPARD, ACADVL, ACAT1, DGAT1, PPARGC1A, ESRRA, CPT1A/1B, or any combination thereof. In an aspect, maturation of iPSC-CMs improved or enhanced by a disclosed contacting or a disclosed transfection or a disclosed transduction can be assessed by a decrease in the expression level of ALDOA, HK1, HK2, PGK1, GAPDH, LDHA, or any combination thereof.

In an aspect, maturation of iPSC-CMs improved or enhanced by a disclosed contacting or a disclosed transfection or a disclosed transduction can be assessed by an increase in mitochondrial numbers and size, an increase in the number of peri-sarcomeric mitochondria, a decrease in the number of perinuclear mitochondria, or any combination thereof.

In an aspect, maturation of iPSC-CMs improved or enhanced by a disclosed contacting or a disclosed transfection or a disclosed transduction can be assessed by an increase in the expression of genes encoding electronic transport chain (ETC) proteins.

In an aspect, maturation of iPSC-CMs improved or enhanced by a disclosed contacting or a disclosed transfection or a disclosed transduction can be assessed by a reduction in glucose uptake, glycogen storage, lactate production, hexokinase activity, the proportion of glycolysis-related ATP production, or any combination thereof.

In an aspect, maturation of iPSC-CMs improved or enhanced by a disclosed contacting or a disclosed transfection or a disclosed transduction can be assessed by an increase in the relative contribution of fatty acid oxidation (FAO) to ATP production.

In an aspect, a disclosed contacting or a disclosed transfection or a disclosed transduction can be used to enhance or increase or promoter the metabolic maturation of iPSC-CMs (e.g., improved mitochondrial structure and function, decreased glycolytic activity, increased FAO, increased ATP production, or any combination thereof).

In an aspect, a disclosed contacting or a disclosed transfection or a disclosed transduction can be used to enhance or increase or promote maturation of iPSC-CMs, which can be used to investigate cardiomyopathy disease mechanisms or can be used to interrogate cardiac disease modeling or can be used to screen drugs for efficacy and/or safety.

For example, a drug can be screened against cardiomyocytes and/or cardiac organoids to determine general cardiotoxicity, or to determine whether cardiomyocytes and/or cardiac organoids obtained from progenitor cells of a particular individual display sensitivity to possible cardiotoxic drugs or other molecules or compounds.

In an aspect, a disclosed contacting or a disclosed transfection or a disclosed transduction can be used to enhance or increase or promote maturation of iPSC-CMs as measured and/or assessed by contractile force, morphology, electrophysiology, calcium handling characteristics, metabolic profile, or any combination thereof.

In an aspect, maturation of iPSC-CMs enhanced or increased or promoted by a disclosed contacting or a disclosed transfection or a disclosed transduction can be assessed by beating when stimulated with a force closer to around 40-80 mN/mm² (e.g., indicative of mature CMs) rather than a force closer to around 0.08-4 mN/mm² (e.g., indicative of immature hiPSC-CMs). In an aspect, maturation of iPSC-CMs enhanced or increased or promoted by a disclosed contacting or a disclosed transfection or a disclosed transduction can be assessed by a conduction velocity around 60 cm/s (e.g., indicative of mature CMs) rather than a conduction velocity closer to around 10-20 cm/s (e.g., indicative of immature hiPSC-CMs). In an aspect, maturation of iPSC-CMs enhanced or increased or promoted by a disclosed contacting or a disclosed transfection or a disclosed transduction can be assessed by beating when stimulated with an upstroke velocity about 150-350 V/s (e.g., indicative of mature CMs) rather than an upstroke velocity closer to around 10-50 V/s (e.g., indicative of immature hiPSC-CMs).

In an aspect, a disclosed iPSC-CM or disclosed iPSC-CMs can be administered to a subject in need thereof. In an aspect, a disclosed subject can have one or more cardiac diseases or disorders.

In an aspect, a disclosed cardiac disease or disorder can comprise myocardial infarction or ischemic cardiomyopathy. In an aspect, a disclosed cardiac disease or disorder can comprise heart failure. In an aspect, a disclosed cardiac disease or disorder can comprise coronary artery disease or an arrhythmia. Cardiac diseases and disorders are known to the skilled person in the art. In an aspect, a disclosed iPSC-CM or disclosed iPSC-CMs can be added to an implantable cardiac patch. Cardiac patches are known to the art. In an aspect, a disclosed implantable cardiac patch can be provided to a subject in need thereof.

In an aspect, a disclosed iPSC-CM or disclosed iPSC-CMs can slow or prevent progression of a cardiac disease or disorder in a subject in need thereof. In an aspect, slowing or preventing disease progression or a cardiac disease or disorder can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cardiac disease or disorder; (vii) reversing, inhibiting, preventing, stabilizing, and/or slowing the rate of progression of a cardiac disease or disorder, or (viii) any combination thereof.

In an aspect, a disclosed iPSC-CM can demonstrate a reduced intensity and/or duration of an arrhythmic event or the number or frequency of arrhythmic events.

In an aspect, iPSc-CMs can be matured into CMs or more matured CMs when compared to the CMs prior to contact with the disclosed viral vector or disclosed non-viral vector. In an aspect, matured CMs can be used in several applications. For example, CMs matured by a disclosed viral vector or disclosed non-viral vector or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, matured CMs can demonstrate upregulated expression of ion channels (e.g., KCNJ2, KCNH2, GJA1, etc.) indicative of adult CMs and can be used for drug evaluation (e.g., efficacy, toxicity, safety, etc.). In an aspect, the upregulation of ion channel expression in matured CMs can be a useful tool in the development of relevant cardiac therapies. In an aspect, matured CMs can be helpful in ensuring that a drug is non-toxic and does not have a proarrhythmic effects prior to administration to a subject in need thereof. In an aspect, matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stereolithography, inkjet, etc.). In an aspect, matured CMs can be used in a disclosed therapeutic application, for example, an application to treat the disease progression of a subject having a diseased or damaged heart. In an aspect, matured CMs demonstrate stabilized repolarization, upregulated conduction velocity, stabilized resting membrane potential, reduced spontaneous firing, or any combination thereof.

### 5. Pharmaceutical Formulations

Disclosed herein is a pharmaceutical formulation comprising a disclosed nucleic acid molecule and a pharmaceutically acceptable carrier.

Disclosed herein is a pharmaceutical formulation comprising a nucleic acid molecule comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier. Disclosed herein is a pharmaceutical formulation comprising a nucleic acid molecule comprising a nucleic acid sequence encoding a Zim3-KRAB-dCas9 effector and a pharmaceutically acceptable carrier. Disclosed herein is a pharmaceutical formulation comprising a nucleic acid molecule comprising a nucleic acid sequence encoding a sequence encoding a Zim3-KRAB-MeCP2-dCas9 effector and a pharmaceutically acceptable carrier.

Disclosed herein is a pharmaceutical formulation comprising a disclosed viral vector and a pharmaceutically acceptable carrier. Disclosed herein is a pharmaceutical formulation comprising a disclosed non-viral vector and a pharmaceutically acceptable carrier. Disclosed herein is a pharmaceutical formulation comprising a disclosed viral vector comprising a disclosed nucleic acid molecule and a pharmaceutically acceptable carrier. Disclosed herein is a pharmaceutical formulation comprising a disclosed non-viral vector comprising a disclosed nucleic acid molecule and a pharmaceutically acceptable carrier.

Disclosed herein is a pharmaceutical formulation comprising a disclosed viral vector comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier. Disclosed herein is a pharmaceutical formulation comprising a disclosed viral vector comprising a nucleic acid sequence encoding a Zim3-KRAB-dCas9 effector and a pharmaceutically acceptable carrier. Disclosed herein is a pharmaceutical formulation comprising a disclosed viral vector comprising a nucleic acid sequence encoding a Zim3-KRAB-MeCP2-dCas9 effector and a pharmaceutically acceptable carrier.

Disclosed herein is a pharmaceutical formulation comprising a disclosed non-viral vector comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier. Disclosed herein is a pharmaceutical formulation comprising a disclosed non-viral vector comprising a nucleic acid sequence encoding a Zim3-KRAB-dCas9 effector and a pharmaceutically acceptable carrier. Disclosed herein is a pharmaceutical formulation comprising a disclosed non-viral vector comprising a nucleic acid sequence encoding a Zim3-KRAB-MeCP2-dCas9 effector and a pharmaceutically acceptable carrier.

Disclosed herein is a pharmaceutical formulation comprising a disclosed recombinant viral vector comprising a disclosed nucleic acid molecule and a pharmaceutically acceptable carrier. Disclosed herein is a pharmaceutical formulation comprising a disclosed recombinant viral vector comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier. Disclosed herein is pharmaceutical formulation comprising a disclosed recombinant viral vector comprising a nucleic acid sequence encoding a Zim3-KRAB-MeCP2-dCas9 effector and a pharmaceutically acceptable carrier.

Disclosed herein is a pharmaceutical formulation comprising a disclosed recombinant non-viral vector comprising a disclosed nucleic acid molecule and a pharmaceutically acceptable carrier. Disclosed herein is a pharmaceutical formulation comprising a disclosed recombinant non-viral vector comprising a nucleic acid sequence encoding (i) at least one zinc finger protein, (ii) at least one polypeptide having effector activity, and (iii) a deactivated Cas9 (dCas9) endonuclease and a pharmaceutically acceptable carrier.

Disclosed herein is pharmaceutical formulation comprising a disclosed recombinant non-viral vector comprising a nucleic acid sequence encoding a Zim3-KRAB-MeCP2-dCas9 effector and a pharmaceutically acceptable carrier. Disclosed herein is a pharmaceutical formulation comprising one or more iPSC-CMs contacted with a disclosed nucleic acid molecule, a disclosed viral vector, a disclosed non-viral vector, a disclosed plasmid, a disclosed pharmaceutical formulation, or any combination. Disclosed herein is a pharmaceutical formulation comprising one or more iPSC-CMs transfected with a disclosed nucleic acid molecule, a disclosed non-viral vector, a disclosed plasmid, a pharmaceutical formulation, or any combination. Disclosed herein is a pharmaceutical formulation comprising one or more iPSC-CMs transduced with a disclosed viral vector or a pharmaceutical formulation.

Disclosed herein is a pharmaceutical formulation comprising iPSC-CMs transduced by a disclosed nucleic acid molecule. Disclosed herein is a pharmaceutical formulation comprising iPSC-CMs transduced by a nucleic acid molecule comprising a nucleic acid sequence encoding (i) at least one zinc finger protein, (ii) at least one polypeptide having effector activity, and (iii) a deactivated Cas9 (dCas9) endonuclease. Disclosed herein is a pharmaceutical formulation comprising iPSC-CMs transduced by a nucleic acid molecule comprising a sequence encoding a Zim3-KRAB-dCas9 effector. Disclosed herein is an iPSC-CMs transduced by a nucleic acid molecule comprising a sequence encoding a Zim3-KRAB-MeCP2-dCas9 effector. Disclosed herein is a pharmaceutical formulation comprising iPSC-CMs transfected by a disclosed viral vector.

Disclosed herein is a pharmaceutical formulation comprising iPSC-CMs transfected by a viral vector comprising a nucleic acid sequence encoding (i) at least one zinc finger protein, (ii) at least one polypeptide having effector activity, and (iii) a deactivated Cas9 (dCas9) endonuclease. Disclosed herein is a pharmaceutical formulation comprising iPSC-CMs transfected by a viral vector comprising a nucleic acid sequence encoding a Zim3-KRAB-dCas9 effector. Disclosed herein is a pharmaceutical formulation comprising iPSC-CMs transfected by a viral vector comprising a nucleic acid sequence encoding a Zim3-KRAB-MeCP2-dCas9 effector. Disclosed herein is a pharmaceutical formulation comprising iPSC-CMs transfected by a recombinant viral vector comprising a disclosed nucleic acid molecule. Disclosed herein is a pharmaceutical formulation comprising iPSC-CMs transfected by a recombinant viral vector comprising a nucleic acid sequence encoding (i) at least one zinc finger protein, (ii) at least one polypeptide having effector activity, and (iii) a deactivated Cas9 (dCas9) endonuclease. Disclosed herein is a pharmaceutical formulation comprising iPSC-CMs transfected by a recombinant viral vector comprising a nucleic acid sequence encoding a Zim3-KRAB-dCas9 effector. Disclosed herein is a pharmaceutical formulation comprising iPSC-CMs transfected by a recombinant viral vector comprising a nucleic acid sequence encoding a Zim3-KRAB-MeCP2-dCas9 effector.

Disclosed herein is a pharmaceutical formulation comprising iPSC-CMs transduced by a disclosed non-viral vector. Disclosed herein is a pharmaceutical formulation comprising iPSC-CMs transduced by a non-viral vector comprising a nucleic acid sequence encoding (i) at least one zinc finger protein, (ii) at least one polypeptide having effector activity, and (iii) a deactivated Cas9 (dCas9) endonuclease. Disclosed herein is a pharmaceutical formulation comprising iPSC-CMs transduced by a non-viral vector comprising a nucleic acid sequence encoding a Zim3-KRAB-dCas9 effector. Disclosed herein is an iPSC-CMs transduced by a non-viral vector comprising a nucleic acid sequence encoding a Zim3-KRAB-MeCP2-dCas9 effector. Disclosed herein is a pharmaceutical formulation comprising iPSC-CMs transduced by a recombinant non-viral vector comprising a disclosed nucleic acid molecule. Disclosed herein is a pharmaceutical formulation comprising iPSC-CMs transduced by a recombinant non-viral vector comprising a nucleic acid sequence encoding (i) at least one zinc finger protein, (ii) at least one polypeptide having effector activity, and (iii) a deactivated Cas9 (dCas9) endonuclease. Disclosed herein is a pharmaceutical formulation comprising iPSC-CMs transduced by a recombinant non-viral vector comprising a nucleic acid sequence encoding a Zim3-KRAB-dCas9 effector. Disclosed herein is a pharmaceutical formulation comprising iPSC-CMs transduced by a recombinant non-viral vector comprising a nucleic acid sequence encoding a Zim3-KRAB-MeCP2-dCas9 effector.

In an aspect, a disclosed pharmaceutical formulation can improve or enhancing maturation of iPSC-CMs. In an aspect, a disclosed pharmaceutical formulation can demonstrate an increased expression and/or activity level when compared to a wild-type expression level or control expression level, or when compared to the expression level in the absence of the disclosed pharmaceutical formulation. In an aspect, a disclosed pharmaceutical formulation can demonstrate a decreased expression and/or activity level when compared to a wild-type expression level or control expression level, or when compared to the expression level in the absence of the disclosed pharmaceutical formulation. In an aspect, a disclosed pharmaceutical formulation can increase the expression and/or activity level of one or more cardiac ion channels when compared to a wild-type expression level or control expression level, or when compared to the expression level in the absence of the disclosed pharmaceutical formulation.

In an aspect, a disclosed pharmaceutical formulation can increase the expression level and/or activity level of one or more of KCNJ2, KCNH2, GJA1, or any combination thereof, or when compared to the expression level in the absence of the disclosed pharmaceutical formulation. In an aspect, a disclosed pharmaceutical formulation can enhance or improve the functionality of one or more cardiac ion channels when compared to a wild-type expression level or control expression level, or when compared to the expression level in the absence of the disclosed pharmaceutical formulation. In an aspect, a disclosed pharmaceutical formulation can enhance or improve the functionality of KCNJ2, KCNH2, GJA1, or any combination thereof, or when compared to the expression level in the absence of the disclosed pharmaceutical formulation. In an aspect, a disclosed pharmaceutical formulation can improve or enhance the functionality of one or more cardiac ion channels when compared to a wild-type expression level or control expression level, or when compared to the expression level in the absence of the disclosed pharmaceutical formulation. In an aspect, a disclosed pharmaceutical formulation can improve or enhance the functionality of KCNJ2, KCNH2, GJA1, or any combination thereof, or when compared to the expression level in the absence of the disclosed pharmaceutical formulation.

In an aspect, a disclosed pharmaceutical formulation can drive or stimulate one or more electrophysiological changes indicative of a mature phenotype in iPSC-CMs when compared to a wild-type expression level or control expression level, or when compared to the expression level in the absence of the disclosed pharmaceutical formulation. In an aspect, a disclosed pharmaceutical formulation can drive or stimulate one or more electrophysiological changes (e.g., increase in expression of KCNJ2, KCNH2, GJA1, or any combination thereof) indicative of a mature phenotype in iPSC-CMs, or when compared to the expression level in the absence of the disclosed pharmaceutical formulation.

In an aspect, matured iPSC-CMs can be characterized by mitochondrial maturation, increased oxidative capacity and enhanced fatty acid use for energy production. For example, the structural, electrophysiological, contractile, and metabolic characteristics of iPSC-CMs can be under-developed when compared to adulty cardiomyocytes. In an aspect, a disclosed pharmaceutical formulation can be used to generate a mature structural, electrophysiological, contractile, and metabolic profile.

In an aspect, maturation of iPSC-CMs improved or enhanced by a disclosed pharmaceutical formulation can be assessed by an increase in the expression level of PDK4, CD36, PPARA, ATP5, LPL, SCD, PPARD, ACADVL, ACAT1, DGAT1, PPARGC1A, ESRRA, N2B, CAV3, SERCA2, CPT1A/1B, or any combination thereof. In an aspect, maturation of iPSC-CMs improved or enhanced by a disclosed pharmaceutical formulation can be assessed by a decrease in the expression level of ALDOA, HK1, HK2, PGK1, GAPDH, LDHA, or any combination thereof.

In an aspect, maturation of iPSC-CMs improved or enhanced by a disclosed pharmaceutical formulation can be assessed by an increase in mitochondrial numbers and size, an increase in the number of peri-sarcomeric mitochondria, a decrease in the number of perinuclear mitochondria, or any combination thereof.

In an aspect, maturation of iPSC-CMs improved or enhanced by a disclosed pharmaceutical formulation can be assessed by an increase in the expression of genes encoding electronic transport chain (ETC) proteins.

In an aspect, maturation of iPSC-CMs improved or enhanced by a disclosed pharmaceutical formulation can be assessed by a reduction in glucose uptake, glycogen storage, lactate production, hexokinase activity, the proportion of glycolysis-related ATP production, or any combination thereof.

In an aspect, maturation of iPSC-CMs improved or enhanced by a disclosed pharmaceutical formulation can be assessed by an increase in the relative contribution of fatty acid oxidation (FAO) to ATP production.

In an aspect, a disclosed pharmaceutical formulation can be used to enhance or increase or promoter the metabolic maturation of iPSC-CMs (e.g., improved mitochondrial structure and function, decreased glycolytic activity, increased FAO, increased ATP production, or any combination thereof).

In an aspect, a disclosed pharmaceutical formulation can be used to enhance or increase or promote maturation of iPSC-CMs, which can be used to investigate cardiomyopathy disease mechanisms or can be used to interrogate cardiac disease modeling or can be used to screen drugs for efficacy and/or safety.

In an aspect, a disclosed pharmaceutical formulation can be used to enhance or increase or promote maturation of iPSC-CMs as measured and/or assessed by contractile force, morphology, electrophysiology, calcium handling characteristics, metabolic profile, or any combination thereof.

In an aspect, maturation of iPSC-CMs enhanced or increased or promoted by a disclosed pharmaceutical formulation can be assessed by beating when stimulated with a force closer to around 40-80 mN/mm² (e.g., indicative of mature CMs) rather than a force closer to around 0.08-4 mN/mm² (e.g., indicative of immature hiPSC-CMs). In an aspect, maturation of iPSC-CMs enhanced or increased or promoted by a disclosed pharmaceutical formulation can be assessed by a conduction velocity around 60 cm/s (e.g., indicative of mature CMs) rather than a conduction velocity closer to around 10-20 cm/s (e.g., indicative of immature hiPSC-CMs). In an aspect, maturation of iPSC-CMs enhanced or increased or promoted by a disclosed pharmaceutical formulation can be assessed by beating when stimulated with an upstroke velocity about 150-350 V/s (e.g., indicative of mature CMs) rather than an upstroke velocity closer to around 10-50 V/s (e.g., indicative of immature hiPSC-CMs).

In an aspect, a disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise an RNA therapeutic. An RNA therapeutic can comprise RNA-mediated interference (RNAi) and/or antisense oligonucleotides (ASO). In an aspect, a disclosed formulation can comprise a disclosed small molecule.

In an aspect, a disclosed pharmaceutical formulation can mature one or more iPSC-CMs.

In an aspect, a disclosed pharmaceutical formulation can be administered to a subject in need thereof. In an aspect, a disclosed subject can have one or more cardiac diseases or disorders. In an aspect, a disclosed cardiac disease or disorder can comprise myocardial infarction or ischemic cardiomyopathy. In an aspect, a disclosed cardiac disease or disorder can comprise heart failure. In an aspect, a disclosed cardiac disease or disorder can comprise coronary artery disease or an arrhythmia. Cardiac diseases and disorders are known to the skilled person in the art. In an aspect, a disclosed pharmaceutical formulation can be added to an implantable cardiac patch. Cardiac patches are known to the art. In an aspect, a disclosed implantable cardiac patch can be provided to a subject in need thereof.

In an aspect, a disclosed pharmaceutical formulation can slow or prevent progression of a cardiac disease or disorder in a subject in need thereof. In an aspect, slowing or preventing disease progression or a cardiac disease or disorder can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cardiac disease or disorder; (vii) reversing, inhibiting, preventing, stabilizing, and/or slowing the rate of progression of a cardiac disease or disorder, or (viii) any combination thereof.

In an aspect, a disclosed pharmaceutical formulation can decrease or minimize the intensity and/or duration of an arrhythmic event or the number or frequency of arrhythmic events.

In an aspect, a disclosed pharmaceutical formulation can be used to transform iPSc-CMs into matured CMs or more matured CMs when compared to the CMs prior to contact with the disclosed pharmaceutical formulation. In an aspect, matured CMs can be used in several applications. For example, CMs matured by a disclosed pharmaceutical formulation or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, a disclosed pharmaceutical formulation can be used to upregulate the expression of ion channels (e.g., KCNJ2, KCNH2, GJA1, etc.) indicative of adult CMs for use in drug evaluation (e.g., efficacy, toxicity, safety, etc.). In an aspect, the upregulation of ion channel expression in matured CMs can be a useful tool in the development of relevant cardiac therapies. In an aspect, matured CMs can be helpful in ensuring that a drug is non-toxic and does not have a proarrhythmic effects prior to administration to a subject in need thereof. In an aspect, matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stereolithography, inkjet, etc.). In an aspect, matured CMs can be used in a disclosed therapeutic application, for example, an application to treat the disease progression of a subject having a diseased or damaged heart. In an aspect, a disclosed pharmaceutical formulation can be used stabilize repolarization, upregulate conduction velocity, stabilize the resting membrane potential, reduce spontaneous firing, or any combination thereof. In an aspect, matured CMs can demonstrate stabilized repolarization, upregulated conduction velocity, stabilized the resting membrane potential, reduced spontaneous firing, or any combination thereof.

### 6. Cardiac Patches

Disclosed herein is an implantable cardiac patch comprising one or more disclosed iPSC-CMs. Disclosed herein is an implantable cardiac patch comprising a disclosed nucleic acid molecule, a disclosed viral vector, a disclosed non-viral vector, a disclosed plasmid, a disclosed pharmaceutical formulation, or any combination. Disclosed herein is an implantable cardiac patch comprising one or more iPSC-CMs transfected with a disclosed nucleic acid molecule, a disclosed non-viral vector, a disclosed plasmid, a pharmaceutical formulation, or any combination. Disclosed herein is an implantable cardiac patch comprising one or more iPSC-CMs transduced with a disclosed viral vector or a pharmaceutical formulation.

Disclosed herein is an implantable cardiac patch comprising one or more iPSC-CMs transduced by a disclosed nucleic acid molecule. Disclosed herein is an implantable cardiac patch comprising one or more iPSC-CMs transduced by a nucleic acid molecule comprising a nucleic acid sequence encoding (i) at least one zinc finger protein, (ii) at least one polypeptide having effector activity, and (iii) a deactivated Cas9 (dCas9) endonuclease. Disclosed herein is an implantable cardiac patch comprising one or more iPSC-CMs transduced by a nucleic acid molecule comprising a sequence encoding a Zim3-KRAB-dCas9 effector. Disclosed herein is an implantable cardiac patch comprising one or more iPSC-CMs transduced by a nucleic acid molecule comprising a sequence encoding a Zim3-KRAB-MeCP2-dCas9 effector.

Disclosed herein is an implantable cardiac patch comprising one or more iPSC-CMs transfected by a disclosed viral vector. Disclosed herein is an implantable cardiac patch comprising one or more iPSC-CMs transfected by a viral vector comprising a nucleic acid sequence encoding (i) at least one zinc finger protein, (ii) at least one polypeptide having effector activity, and (iii) a deactivated Cas9 (dCas9) endonuclease. Disclosed herein is an implantable cardiac patch comprising one or more iPSC-CMs transfected by a viral vector comprising a nucleic acid sequence encoding a Zim3-KRAB-dCas9 effector. Disclosed herein is an implantable cardiac patch comprising one or more iPSC-CMs transfected by a viral vector comprising a nucleic acid sequence encoding a Zim3-KRAB-MeCP2-dCas9 effector. Disclosed herein is an implantable cardiac patch comprising one or more iPSC-CMs transfected by a recombinant viral vector comprising a disclosed nucleic acid molecule. Disclosed herein is an implantable cardiac patch comprising one or more iPSC-CMs transfected by a recombinant viral vector comprising a nucleic acid sequence encoding (i) at least one zinc finger protein, (ii) at least one polypeptide having effector activity, and (iii) a deactivated Cas9 (dCas9) endonuclease. Disclosed herein is an implantable cardiac patch comprising one or more iPSC-CMs transfected by a recombinant viral vector comprising a nucleic acid sequence encoding a Zim3-KRAB-dCas9 effector. Disclosed herein is an implantable cardiac patch comprising one or more iPSC-CMs transfected by a recombinant viral vector comprising a nucleic acid sequence encoding a Zim3-KRAB-MeCP2-dCas9 effector.

Disclosed herein is an implantable cardiac patch comprising one or more iPSC-CMs transduced by a disclosed non-viral vector. Disclosed herein is an implantable cardiac patch comprising one or more iPSC-CMs transduced by a non-viral vector comprising a nucleic acid sequence encoding (i) at least one zinc finger protein, (ii) at least one polypeptide having effector activity, and (iii) a deactivated Cas9 (dCas9) endonuclease. Disclosed herein is an implantable cardiac patch comprising one or more iPSC-CMs transduced by a non-viral vector comprising a nucleic acid sequence encoding a Zim3-KRAB-dCas9 effector. Disclosed herein is an implantable cardiac patch comprising one or more iPSC-CMs transduced by a non-viral vector comprising a nucleic acid sequence encoding a Zim3-KRAB-MeCP2-dCas9 effector. Disclosed herein is an implantable cardiac patch comprising one or more iPSC-CMs transduced by a recombinant non-viral vector comprising a disclosed nucleic acid molecule. Disclosed herein is an implantable cardiac patch comprising one or more iPSC-CMs transduced by a recombinant non-viral vector comprising a nucleic acid sequence encoding (i) at least one zinc finger protein, (ii) at least one polypeptide having effector activity, and (iii) a deactivated Cas9 (dCas9) endonuclease. Disclosed herein is an implantable cardiac patch comprising one or more iPSC-CMs transduced by a recombinant non-viral vector comprising a nucleic acid sequence encoding a Zim3-KRAB-dCas9 effector. Disclosed herein is an implantable cardiac patch comprising one or more iPSC-CMs transduced by a recombinant non-viral vector comprising a nucleic acid sequence encoding a Zim3-KRAB-MeCP2-dCas9 effector. Disclosed herein is an implantable cardiac patch comprising one or more iPSC-CMs transfected by a disclosed plasmid. Disclosed herein is an implantable cardiac patch comprising one or more iPSC-CMs transfected by a disclosed plasmid comprising a disclosed nucleic acid molecule.

Disclosed herein is an implantable cardiac patch comprising one or more iPSC-CMs transfected by a disclosed plasmid comprising the sequence set forth in SEQ ID NO:63 (pHR-UCOE-SFFV-dCas9-mCherry-Zim3-KRAB) or SEQ ID NO:64 (pAV[Exp]-mCherry-SFFV-dCas9/KRAB/MeCP2).

Disclosed herein is an implantable cardiac patch comprising one or more iPSC-CMs transfected by a plasmid comprising a nucleic acid sequence encoding (i) at least one zinc finger protein, (ii) at least one polypeptide having effector activity, and (iii) a deactivated Cas9 (dCas9) endonuclease. Disclosed herein is an implantable cardiac patch comprising one or more iPSC-CMs transfected by a plasmid comprising a nucleic acid sequence encoding a Zim3-KRAB-dCas9 effector. Disclosed herein is an implantable cardiac patch comprising one or more iPSC-CMs transfected by a plasmid comprising a nucleic acid sequence encoding a Zim3-KRAB-MeCP2-dCas9 effector.

In an aspect, a disclosed implantable cardiac patch can comprise iPSC-CMs that demonstrate improved or enhanced maturation. In an aspect, following either (i) the transfection of the iPSC-CMs with a disclosed nucleic acid molecule, a disclosed non-viral vector, a disclosed plasmid, a pharmaceutical formulation, or any combination, or (ii) the transduction of the iPSC-CMs with a disclosed viral vector or a pharmaceutical formulation, the iPSC-CMs can demonstrate improved or enhanced maturation.

In an aspect of a disclosed cardiac patch, following a disclosed contacting or a disclosed transfection or a disclosed transduction of the iPSC-CMs, the functionality of one or more cardiac ion channels can be improved or enhanced when compared to a wild-type expression level or control expression level, or when compared to the expression level in the absence of the contacting or the transfection or transduction. In an aspect, iPSC-CMs demonstrating improved or enhanced maturation are discussed supra (for example, in Section VI(B)(4)). In an aspect, matured iPSC-CMs can be characterized by mitochondrial maturation, increased oxidative capacity, and enhanced fatty acid use for energy production. For example, the structural, electrophysiological, contractile, and metabolic characteristics of the iPSC-CMs can be under-developed when compared to adult cardiomyocytes. In an aspect, a disclosed contacting or a disclosed transfection or a disclosed transduction of the iPSC-CMs can be used to generate a mature structural, electrophysiological, contractile, and metabolic profile.

In an aspect, maturation of iPSC-CMs can be assessed by an increase in the expression level of PDK4, CD36, PPARA, ATP5, LPL, SCD, PPARD, ACADVL, ACAT1, DGAT1, PPARGC1A, ESRRA, CPT1A/1B, or any combination thereof. In an aspect, maturation of iPSC-CMs can be assessed by a decrease in the expression level of ALDOA, HK1, HK2, PGK1, GAPDH, LDHA, or any combination thereof. In an aspect, maturation of iPSC-CMs can be assessed by an increase in mitochondrial numbers and size, an increase in the number of peri-sarcomeric mitochondria, a decrease in the number of perinuclear mitochondria, or any combination thereof. In an aspect, maturation of iPSC-CMs can be assessed by an increase in the expression of genes encoding electronic transport chain (ETC) proteins. n an aspect, maturation of iPSC-CMs can be assessed by a reduction in glucose uptake, glycogen storage, lactate production, hexokinase activity, the proportion of glycolysis-related ATP production, or any combination thereof. In an aspect, maturation of iPSC-CMs can be assessed by an increase in the relative contribution of fatty acid oxidation (FAO) to ATP production. In an aspect, maturation of iPSC-CMs can be assessed by improved mitochondrial structure and function, decreased glycolytic activity, increased FAO, increased ATP production, or any combination thereof.

In an aspect, maturation of iPSC-CMs can be assessed by beating when stimulated with a force closer to around 40-80 mN/mm² (e.g., indicative of mature CMs) rather than a force closer to around 0.08-4 mN/mm² (e.g., indicative of immature hiPSC-CMs). In an aspect, maturation of iPSC-CMs can be assessed by a conduction velocity around 60 cm/s (e.g., indicative of mature CMs) rather than a conduction velocity closer to around 10-20 cm/s (e.g., indicative of immature hiPSC-CMs). In an aspect, maturation of iPSC-CMs can be assessed by beating when stimulated with an upstroke velocity about 150-350 V/s (e.g., indicative of mature CMs) rather than an upstroke velocity closer to around 10-50 V/s (e.g., indicative of immature hiPSC-CMs).

In an aspect, a disclosed implantable cardiac patch can slow or prevent progression of a cardiac disease or disorder in a subject in need thereof. In an aspect, slowing or preventing disease progression or a cardiac disease or disorder can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cardiac disease or disorder; (vii) reversing, inhibiting, preventing, stabilizing, and/or slowing the rate of progression of a cardiac disease or disorder, or (viii) any combination thereof.

In an aspect, a disclosed implantable cardiac patch can decrease or minimize the intensity and/or duration of an arrhythmic event or the number or frequency of arrhythmic events. In an aspect, a disclosed implantable cardiac patch can be used in a method of slowing and/or preventing progression of a cardiac disease or cardiac disorder.

In an aspect, a disclosed implantable cardiac patch can comprise matured CMs or more matured CMs when compared to the CMs prior to contact with the disclosed composition. In an aspect, matured CMs can be used in several applications. For example, CMs matured by a disclosed composition or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, a disclosed implantable cardiac patch can demonstrate upregulated expression of ion channels (e.g., KCNJ2, KCNH2, GJA1, etc.) indicative of adult CMs for use in drug evaluation (e.g., efficacy, toxicity, safety, etc.). In an aspect, the upregulation of ion channel expression in matured CMs can be a useful tool in the development of relevant cardiac therapies. In an aspect, a disclosed implantable cardiac patch comprising matured CMs can be helpful in ensuring that a drug is non-toxic and does not have a proarrhythmic effects prior to administration to a subject in need thereof. In an aspect, a disclosed implantable cardiac patch comprising matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stereolithography, inkjet, etc.). In an aspect, a disclosed implantable cardiac patch comprising matured CMs can be used in a disclosed therapeutic application, for example, an application to treat the disease progression of a subject having a diseased or damaged heart. In an aspect, a disclosed implantable cardiac patch comprising matured CMs can demonstrate stabilized repolarization, upregulated conduction velocity, stabilized the resting membrane potential, reduced spontaneous firing, or any combination thereof.

### C. Methods of Improving or Enhancing Maturation of iPSC-CMs

Disclosed herein is a method of improving or enhancing maturation of iPSC-CMs, the method comprising contacting one or more iPSC-CMs with a therapeutically effective amount of a disclosed nucleic acid molecule, wherein following the contacting step, the one or more iPSC-CMs are characterized by a mature structural, electrophysiological, contractile, and metabolic profile. Disclosed herein is a method of improving or enhancing maturation of iPSC-CMs, the method comprising contacting one or more iPSC-CMs with a therapeutically effective amount of a disclosed viral or non-viral vector, wherein following the contacting step, the one or more iPSC-CMs are characterized by a mature structural, electrophysiological, contractile, and metabolic profile.

Disclosed herein is a method of improving or enhancing maturation of iPSC-CMs, the method comprising contacting one or more iPSC-CMs with a therapeutically effective amount of a disclosed plasmid, wherein following the contacting step, the one or more iPSC-CMs are characterized by a mature structural, electrophysiological, contractile, and metabolic profile.

In an aspect, iPSC-CMs can be human iPSC-CMs or non-human iPSC-CMs.

In an aspect of a disclosed method, matured iPSC-CMs can be characterized by mitochondrial maturation, increased oxidative capacity, and enhanced fatty acid use for energy production. For example, the structural, electrophysiological, contractile, and metabolic characteristics of iPSC-CMs can be under-developed when compared to adulty cardiomyocytes. In an aspect, a disclosed method can be used to generate a mature structural, electrophysiological, contractile, and metabolic profile.

In an aspect, maturation of iPSC-CMs improved or enhanced by a disclosed method can be assessed by an increase in the expression level of PDK4, CD36, PPARA, ATP5, LPL, SCD, PPARD, ACADVL, ACAT1, DGAT1, PPARGC1A, ESRRA, N2B, CAV3, SERCA2, CPT1A/1B, or any combination thereof. In an aspect, maturation of iPSC-CMs improved or enhanced by a disclosed method can be assessed by a decrease in the expression level of ALDOA, HK1, HK2, PGK1, GAPDH, LDHA, or any combination thereof.

In an aspect, maturation of iPSC-CMs improved or enhanced by a disclosed method can be assessed by an increase in mitochondrial numbers and size, an increase in the number of peri-sarcomeric mitochondria, a decrease in the number of perinuclear mitochondria, or any combination thereof. In an aspect, maturation of iPSC-CMs improved or enhanced by a disclosed method can be assessed by an increase in the expression of genes encoding electronic transport chain (ETC) proteins.

In an aspect, maturation of iPSC-CMs improved or enhanced by a disclosed method can be assessed by a reduction in glucose uptake, glycogen storage, lactate production, hexokinase activity, the proportion of glycolysis-related ATP production, or any combination thereof. In an aspect, maturation of iPSC-CMs improved or enhanced by a disclosed method can be assessed by an increase in the relative contribution of fatty acid oxidation (FAO) to ATP production. In an aspect, a disclosed method can be used to enhance or increase or promote the metabolic maturation of iPSC-CMs (e.g., improved mitochondrial structure and function, decreased glycolytic activity, increased FAO, increased ATP production, or any combination thereof).

In an aspect, a disclosed method can be used to enhance or increase or promote maturation of iPSC-CMs, which can be used to investigate cardiomyopathy disease mechanisms or can be used to interrogate cardiac disease modeling or can be used to screen drugs for efficacy and/or safety.

For example, a drug can be screened against cardiomyocytes and/or cardiac organoids to determine general cardiotoxicity, or to determine whether cardiomyocytes and/or cardiac organoids obtained from progenitor cells of a particular individual display sensitivity to possible cardiotoxic drugs or other molecules or compounds.

In an aspect, a disclosed method can be used to enhance or increase or promote maturation of iPSC-CMs as measured and/or assessed by contractile force, morphology, electrophysiology, calcium handling characteristics, metabolic profile, or any combination thereof.

In an aspect, maturation of iPSC-CMs enhanced or increased or promoted a disclosed method can be assessed by beating when stimulated with a force closer to around 40 mN/mm²-80 mN/mm² (e.g., indicative of mature CMs) rather than a force closer to around 0.08 mN/mm²-4 mN/mm² (e.g., indicative of immature hiPSC-CMs). In an aspect, maturation of iPSC-CMs enhanced or increased or promoted by a disclosed method can be assessed by a conduction velocity around 60 cm/s (e.g., indicative of mature CMs) rather than a conduction velocity closer to around 10-20 cm/s (e.g., indicative of immature hiPSC-CMs). In an aspect, maturation of iPSC-CMs enhanced or increased or promoted by a disclosed method can be assessed by beating when stimulated with an upstroke velocity about 150 V/s-350 V/s (e.g., indicative of mature CMs) rather than an upstroke velocity closer to around 10 V/s-50 V/s (e.g., indicative of immature hiPSC-CMs).

In an aspect, a disclosed method improving or enhancing maturation of iPSC-CMs can further comprise generating a disclosed nucleic acid molecule, a disclosed viral vector or non-viral vector, a disclosed plasmid, a disclosed pharmaceutical formulation, or any combination thereof.

In an aspect, a disclosed method can further comprise culturing one or more disclosed iPSC-CMs. In an aspect, iPSC-CMs can be cultured according to one or more methods known to the art. For example, in an aspect, it can be good practice to observe iPSC lines daily under phase contrast microscopy (e.g., 4×, 10×, 20× and 40× magnification) to check for iPSC-like morphology, the presence of differentiated cells and confluence. A typical scoring can be outline as (i) optimal, compacted iPSC colonies with defined edges; morphology uniform across colonies, (ii) acceptable iPSC colonies with some differentiation around the edges, cells more loosely packed within colonies, (iii) good adherence with small iPSCs colonies emerging, and (iv) poor adherence and no obvious iPSCs. In an aspect, cells can be fed by removing ˜95% of the medium from the wells using an aspirator pipette. In an aspect, the medium cannot be removed completely but rather a thin film of medium can cover the cell layer to avoid drying out the cells. In an aspect, about 2 mL of fresh medium per 1 well of a 6-well plate can be aseptically added by gently adding to the side of the well. Incubate cells at 37° C./5% CO₂. In an aspect, a medium exchange can occur daily on six of seven days with increased volume of media (1.5×-2× the normal amount; cell density dependent) if cells need to be left for longer periods between media change. In an aspect, medium exchanges can be effected within 48 hours.

In an aspect, a disclosed method can further comprise using matured iPSC-CMs to treat a disease or disorder (such as, for example, a cardiac disease or disorder). In an aspect, a subject can be a subject in need of treatment of a disclosed disease or disorder (e.g., a cardiac disease or disorder). In an aspect, a disclosed method improving or enhancing maturation of iPSC-CMs can further comprise generating and/or validating one or more of the disclosed isolated nucleic acid molecules, one or more of the disclosed vectors, one or more of the disclosed pharmaceutical formulations, or any combination thereof. In an aspect, a disclosed method improving or enhancing maturation of iPSC-CMs can further comprise contacting the cells with a second disclosed isolated nucleic acid molecule, a second disclosed viral vector or non-viral vector, a second disclosed pharmaceutical formulations, or any combination thereof. In an aspect, a disclosed method improving or enhancing maturation of iPSC-CMs can further comprise contacting the cells with additional disclosed isolated nucleic acid molecules, additional disclosed vectors, additional disclosed pharmaceutical formulations, or any combination thereof.

In an aspect, a disclosed method can further comprise adding matured iPSC-CMs to an implantable cardiac patch. In an aspect, a disclosed implantable patch can be administered to a subject in need thereof. Cardiac patches are known to the art. In an aspect, a disclosed implantable cardiac patch can be provided to a subject in need thereof. In an aspect, administering can comprise grafting or implanting of a disclosed cardiac patch onto one or more portions of a subject's heart. In an aspect, a disclosed subject can have one or more cardiac diseases or disorders. In an aspect, a disclosed cardiac disease or disorder can comprise myocardial infarction or ischemic cardiomyopathy. In an aspect, a disclosed cardiac disease or disorder can comprise heart failure. In an aspect, a disclosed cardiac disease or disorder can comprise coronary artery disease or an arrhythmia. Cardiac diseases and disorders are known to the art.

In an aspect, a disclosed method can further comprise culturing the iPSC-CMs in a fatty acid-based medium. In an aspect, a disclosed method can further comprise culturing in the iPSC-CMs in a three-dimension environment. In an aspect, a disclosed method can further comprise culturing the iPSC-CMs with non-cardiac cells.

In an aspect, a disclosed method can slow or prevent progression of a cardiac disease or disorder in a subject in need thereof. In an aspect, slowing or preventing disease progression or a cardiac disease or disorder can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cardiac disease or disorder; (vii) reversing, inhibiting, preventing, stabilizing, and/or slowing the rate of progression of a cardiac disease or disorder, or (viii) any combination thereof.

In an aspect, a disclosed method can decrease or minimize the intensity and/or duration of an arrhythmic event or the number or frequency of arrhythmic events. In an aspect, a disclosed method can be used in a method of slowing and/or preventing progression of a cardiac disease or cardiac disorder.

In an aspect, a disclosed method can be used to transform iPSc-CMs into matured CMs or more matured CMs when compared to the CMs prior to contact with the disclosed viral vector or disclosed non-viral vector. In an aspect, matured CMs can be used in several applications. For example, CMs matured by a disclosed composition or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, a disclosed method can be used to upregulate the expression of ion channels (e.g., KCNJ2, KCNH2, GJA1, etc.) indicative of adult CMs for use in drug evaluation (e.g., efficacy, toxicity, safety, etc.). In an aspect, the upregulation of ion channel expression in matured CMs can be a useful tool in the development of relevant cardiac therapies. In an aspect, matured CMs can be helpful in ensuring that a drug is non-toxic and does not have a proarrhythmic effects prior to administration to a subject in need thereof. In an aspect, matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stereolithography, inkjet, etc.). In an aspect, matured CMs can be used in a disclosed therapeutic application, for example, an application to treat the disease progression of a subject having a diseased or damaged heart. In an aspect, a disclosed viral vector or disclosed non-viral vector can be used stabilize repolarization, upregulate conduction velocity, stabilize the resting membrane potential, reduce spontaneous firing, or any combination thereof. In an aspect, matured CMs can demonstrate stabilized repolarization, upregulated conduction velocity, stabilized the resting membrane potential, reduced spontaneous firing, or any combination thereof.

### D. Methods of Slowing and/or Preventing Progression of a Cardiac Disease or Disorder

Disclosed herein is a method of slowing and/or preventing progression of a cardiac disease or disorder in a subject, the method comprising administering to a subject a therapeutically effective amount of a disclosed nucleic acid molecule, thereby reducing the pathological phenotype associated with the cardiac disease or disorder. In an aspect, a disclosed nucleic acid molecule is discussed supra, for example, in Section V(B)(1).

Disclosed herein is a method of slowing and/or preventing progression of a cardiac disease or disorder in a subject, the method comprising administering to a subject a therapeutically effective amount of a disclosed viral vector, thereby reducing the pathological phenotype associated with the cardiac disease or disorder. In an aspect, a disclosed viral vector is discussed supra, for example, in Section V(B)(2).

Disclosed herein is a method of slowing and/or preventing progression of a cardiac disease or disorder in a subject, the method comprising administering to a subject a therapeutically effective amount of a disclosed non-viral vector, thereby reducing the pathological phenotype associated with the cardiac disease or disorder. In an aspect, a disclosed non-viral vector is discussed supra, for example, in Section V(B)(1).

Disclosed herein is a method of slowing and/or preventing progression of a cardiac disease or disorder in a subject, the method comprising administering to a subject a therapeutically effective amount of a disclosed pharmaceutical formulation, thereby reducing the pathological phenotype associated with the cardiac disease or disorder. In an aspect, a disclosed pharmaceutical formulation is discussed supra, for example, in Section V(B)(5).

Disclosed herein is a method of slowing and/or preventing progression of a cardiac disease or disorder in a subject, the method comprising administering to a subject a therapeutically effective amount of a disclosed cardiac patch, thereby reducing the pathological phenotype associated with the cardiac disease or disorder. In an aspect, a disclosed cardiac patch is discussed supra, for example, in Section V(B)(6).

Disclosed herein is a method of slowing and/or preventing progression of a cardiac disease or disorder in a subject, the method comprising administering to a subject a therapeutically effective amount of one or more disclosed iPSC-CMs, thereby reducing the pathological phenotype associated with the cardiac disease or disorder. In an aspect, a disclosed nucleic acid molecule is discussed supra, for example, in Section V(B)(4).

In an aspect, a disclosed method can comprise repeating the administering step one or more times.

In an aspect, administering a disclosed cardiac patch can comprise implanting the patch in an affected portion of the subject's heart. In an aspect, administering a disclosed cardiac patch can comprise grafting the patch in or onto an affected portion of the subject's heart. In an aspect, administering a disclosed cardiac patch can comprising overlying the patch onto an affected portion of the subject's heart. In an aspect, an affected portion of the subject's heart can comprise one or more dysfunctional portions of the heart and/or one or more ischemic portions of the heart and/or one or more portions of the heart that has sustained some type of cellular damage.

In an aspect of a disclosed method, a subject can be a human. In an aspect, a subject can be suspected of having or can be diagnosed with having a disclosed cardiac disease or a cardiac disorder.

In an aspect, a disclosed subject can have one or more cardiac diseases or disorders. In an aspect, a disclosed cardiac disease or disorder can comprise myocardial infarction or ischemic cardiomyopathy. In an aspect, a disclosed cardiac disease or disorder can comprise heart failure. In an aspect, a disclosed cardiac disease or disorder can comprise coronary artery disease or an arrhythmia. Cardiac diseases and disorders are known to the skilled person in the art.

In an aspect, a disclosed method can comprise reducing the pathological phenotype associated with a disclosed cardiac disease or a cardiac disorder. In an aspect, a disclosed method can comprise diagnosing the subject with a disclosed cardiac disease or a cardiac disorder. In an aspect, a subject can be a subject in need of treatment of a disclosed cardiac disease or a cardiac disorder. In an aspect, a disclosed subject can be symptomatic or asymptomatic.

In an aspect, a disclosed method of treating and/or preventing a disclosed cardiac disease or a cardiac disorder can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a disclosed cardiac disease or a cardiac disorder; (vii) reversing, inhibiting, preventing, stabilizing, and/or slowing the rate of progression of a disclosed cardiac disease or a cardiac disorder, or (viii) any combination thereof.

In an aspect, restoring one or more aspects of cellular homeostasis can comprise improving, enhancing, restoring, and/or preserving one or more aspects of cellular structural and/or functional integrity. In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is like that of a wild-type or control level (e.g., pre-cardiac disease or pre-cardiac disorder level).

In an aspect of a disclosed method of slowing and/or preventing progression of a cardiac disease or disorder, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method of slowing and/or preventing progression of a cardiac disease or disorder, administering can comprise intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, intrahepatic administration, hepatic intra-arterial administration, hepatic portal vein (HPV) administration, or any combination thereof. In an aspect, a disclosed vector can be administered via LNP administration. In an aspect, administering can comprise grafting or implanting of a disclosed cardiac patch onto one or more portions of a subject's heart.

In an aspect, a disclosed method of slowing and/or preventing progression of a cardiac disease or disorder can employ multiple routes of administration to the subject. In an aspect, a disclosed method can employ a first route of administration that can be the same or different as a second and/or subsequent routes of administration. In an aspect, a disclosed nucleic acid molecule, a disclosed vector, and/or a disclosed pharmaceutical formulation can be concurrently and/or serially administered to a subject via multiple routes of administration. For example, in an aspect, administering a disclosed nucleic acid molecule, a disclosed vector, and/or a disclosed pharmaceutical formulation can comprise intravenous administration and intra-cardiac administration. In an aspect, administering a disclosed nucleic acid molecule, a disclosed vector, and/or a disclosed pharmaceutical formulation can comprise IV administration and intrathecal (ITH) administration. In an aspect, administering can comprise grafting or implanting of a disclosed cardiac patch onto one or more portions of a subject's heart.

In an aspect of a disclosed method of slowing and/or preventing progression of a cardiac disease or disorder, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method of slowing and/or preventing progression of a cardiac disease or disorder can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method of slowing and/or preventing progression of a cardiac disease or disorder can further comprise administering to the subject a therapeutically effective amount of a therapeutic agent. A therapeutic agent can be any disclosed agent that effects a desired clinical outcome.

In an aspect, a disclosed method slowing and/or preventing progression of a cardiac disease or disorder can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method of slowing and/or preventing progression of a cardiac disease or disorder can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus.

In an aspect, a disclosed method of slowing and/or preventing progression of a cardiac disease or disorder can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, a disclosed method slowing and/or preventing progression of a cardiac disease or disorder can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method slowing and/or preventing progression of a cardiac disease or disorder can further comprise administering a compound that exerts a therapeutic effect against B cells and/or a compound that targets or alters antigen presentation or humoral or cell mediated immune response.

In an aspect, a disclosed method can comprise repeating one or more steps of the method and/or modifying one or more steps of the method (such as, for example, an administering step).

In an aspect, a disclosed method of slowing and/or preventing progression of a cardiac disease or disorder can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof administered to a subject, or by changing the frequency of administration of one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof to a subject, or by changing the duration of time one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof are administered to a subject.

In an aspect, a disclosed method of slowing and/or preventing progression of a cardiac disease or disorder can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In an aspect, a disclosed method of slowing and/or preventing progression of a cardiac disease or disorder can further comprise generating and/or validating one or more of the disclosed isolated nucleic acid molecules, one or more of the disclosed vectors, one or more of the disclosed pharmaceutical formulations, or any combination thereof.

In an aspect, a disclosed method of slowing and/or preventing progression of a cardiac disease or disorder can further comprise administering to the subject a second disclosed isolated nucleic acid molecule, a second disclosed vector, a second disclosed pharmaceutical formulations, or any combination thereof. In an aspect, a disclosed method of slowing and/or preventing progression of a cardiac disease or disorder can further comprise administering to the subject additional disclosed isolated nucleic acid molecules, additional disclosed vectors, additional disclosed pharmaceutical formulations, or any combination thereof.

In an aspect, a disclosed method can comprise measuring the subject's progression following the administering step.

In an aspect, a disclosed method can minimize the intensity and/or duration of an arrhythmic event or the number or frequency of arrhythmic events. In an aspect, a disclosed method can be used in a method of slowing and/or preventing progression of a cardiac disease or cardiac disorder.

In an aspect, a disclosed method can be used to transform iPSc-CMs into matured CMs or more matured CMs when compared to the CMs prior to contact with the disclosed viral vector or disclosed non-viral vector. In an aspect, matured CMs can be used in several applications. For example, CMs matured by a disclosed composition or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, a disclosed method can be used to upregulate the expression of ion channels (e.g., KCNJ2, KCNH2, GJA1, etc.) indicative of adult CMs for use in drug evaluation (e.g., efficacy, toxicity, safety, etc.). In an aspect, the upregulation of ion channel expression in matured CMs can be a useful tool in the development of relevant cardiac therapies. In an aspect, matured CMs can be helpful in ensuring that a drug is non-toxic and does not have a proarrhythmic effects prior to administration to a subject in need thereof. In an aspect, matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stereolithography, inkjet, etc.). In an aspect, matured CMs can be used in a disclosed therapeutic application, for example, an application to treat the disease progression of a subject having a diseased or damaged heart. In an aspect, a disclosed viral vector or disclosed non-viral vector can be used stabilize repolarization, upregulate conduction velocity, stabilize the resting membrane potential, reduce spontaneous firing, or any combination thereof. In an aspect, matured CMs can demonstrate stabilized repolarization, upregulated conduction velocity, stabilized the resting membrane potential, reduced spontaneous firing, or any combination thereof.

### E. Kits

Disclosed herein is a kit comprising one or more of the disclosed isolated nucleic acid molecules, disclosed viral vectors, disclosed non-viral vectors, disclosed pharmaceutical formulations, disclosed iPSC-CMs, disclosed cardiac patches, disclosed plasmids, or any combination thereof with or without additional therapeutic agents. In an aspect, a disclosed kit can be used in a disclosed method of improving and/or enhancing maturation of iPSC-CMs or a in disclosed method of slowing and/or preventing progression of a cardiac disease or cardiac disorder.

In an aspect, a disclosed kit can comprise at least two components constituting the kit. Together, the components constitute a functional unit for a given purpose (such as, for example, slowing and/or preventing progression of a cardiac disease or cardiac disorder in a subject). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed pharmaceutical formulation, a disclosed therapeutic agent, a disclosed reagent, or a combination thereof, and a label or package insert with instructions for use. In an aspect, suitable containers include, for example, bottles, vials, syringes, blister pack, etc. The containers can be formed from a variety of materials such as glass or plastic. The container can hold, for example, a disclosed pharmaceutical formulation and/or a disclosed therapeutic agent and can have a sterile access port (for example the container may be an intravenous solution bag or a vial having a stopper pierceable by a hypodermic injection needle). The label or package insert can indicate that a disclosed pharmaceutical formulation and/or a disclosed therapeutic agent can be used for improving and/or enhancing maturation of iPSC-CMs or for slowing and/or preventing progression of a cardiac disease or cardiac disorder. In an aspect, a disclosed kit can comprise additional components necessary for administration such as, for example, other buffers, diluents, filters, needles, and syringes. In an aspect, a disclosed kit can comprise those components (e.g., primers) necessary to measure one or more times the level of expression and/or the level of activity of the disclosed gene of interest.

In an aspect, a disclosed kit can decrease or minimize the intensity and/or duration of an arrhythmic event or the number or frequency of arrhythmic events. In an aspect, a disclosed kit can be used in a method of slowing and/or preventing progression of a cardiac disease or cardiac disorder.

In an aspect, a disclosed kit can be used to transform iPSc-CMs into matured CMs or more matured CMs when compared to the CMs prior to contact with the disclosed viral vector or disclosed non-viral vector. In an aspect, matured CMs can be used in several applications. For example, CMs matured by a disclosed composition or using a disclosed method can be used in drug screening and/or disease modeling. In an aspect, drug screening and/or disease modeling can concern drugs and disease that focus on and/or concern the heart, its anatomy, and/or its function. In an aspect, matured CMs can be used for therapeutic tissue regeneration (such as, for example, cardiac regeneration). For example, in an aspect, matured CMs can be implanted in, grafted onto, and/or injected into the heart of a subject in need thereof. In an aspect, a delivery of matured CMs can be via direct injection into the one or more diseased or damaged parts of a subject's heart. In an aspect, matured CMs can be infused into, added to, grown in, or used in an implantable cardiac patch, which can then be given to a subject in need thereof. Implantable cardiac patches are discussed herein. In an aspect, a disclosed heart can be damaged and/or diseased and can be functioning at a level that compromises and/or negatively affects the quality of a subject's life or the subject's life expectancy. In an aspect, a disclosed kit can be used to upregulate the expression of ion channels (e.g., KCNJ2, KCNH2, GJA1, etc.) indicative of adult CMs for use in drug evaluation (e.g., efficacy, toxicity, safety, etc.). In an aspect, the upregulation of ion channel expression in matured CMs can be a useful tool in the development of relevant cardiac therapies. In an aspect, matured CMs can be helpful in ensuring that a drug is non-toxic and does not have a proarrhythmic effects prior to administration to a subject in need thereof. In an aspect, matured CMs can be used for early detection of drug-related cardiac toxicity and arrhythmogenicity during drug discovery and development. In an aspect, matured CMs can confer consistency and producibility during drug discovery and development, especially when used for early detection of drug-related cardiac toxicity and arrhythmogenicity. In an aspect, matured CMs can be used for bioprinting (e.g., extrusion-based bioprinting, laser-assisted bioprinting, scaffold-free bioprinting, stereolithography, inkjet, etc.). In an aspect, matured CMs can be used in a disclosed therapeutic application, for example, an application to treat the disease progression of a subject having a diseased or damaged heart. In an aspect, a disclosed kit can be used stabilize repolarization, upregulate conduction velocity, stabilize the resting membrane potential, reduce spontaneous firing, or any combination thereof. In an aspect, matured CMs can demonstrate stabilized repolarization, upregulated conduction velocity, stabilized the resting membrane potential, reduced spontaneous firing, or any combination thereof.

TABLE 1
Sequence Information

| SEQ ID # | Name of Sequence | Type | Source |
|---|---|---|---|
| 01 | ZIM3 | AA | human |
| 02 | ZIM3 (mRNA) | NT | human |
| 03 | ZIM3 (genomic) | NT | human |
| 04 | KCNJ2 | AA | human |
| 05 | KCNJ2 (mRNA) | NT | human |
| 06 | KCNJ2 (genomic) | NT | human |
| 07 | KCNH2 | AA | human |
| 08 | KCNH2 (mRNA) | NT | human |
| 09 | KCNH2 (genomic) | NT | human |
| 10 | GJA1 | AA | human |
| 11 | GJA1 (mRNA) | NT | human |
| 12 | GJA1 (genomic) | NT | human |
| 13 | dCas9 | NT | artificial |
| 14 | dSpCas9 | NT | artificial |
| 15 | dSaCas9 | NT | artificial |
| 16 | dSaCas9 | NT | artificial |
| 17 | dCjCas9 | NT | artificial |
| 18 | dCjCas9 | NT | artificial |
| 19 | VRER Cas9 | NT | artificial |
| 20 | dSpCas9 | AA | artificial |
| 21 | dSaCas9 | AA | artificial |
| 22 | dCjCas9 | AA | artificial |
| 23 | ZIM-KRAB | NT | artificial |
| 24 | ZIM-KRAB | AA | artificial |
| 25 | KRAB | AA | artificial |
| 26 | KRAB/MeCP2 | NT | artificial |
| 27 | KRAB/MeCP2 | AA | artificial |
| 28 | KRAB/MeCP2 | AA | artificial |
| 29 | dCas9/KRAB/MeCP2 | NT | artificial |
| 30 | mCherry | NT | artificial |
| 31 | Nuclear Localization Signal | NT | artificial |
| 32 | Nuclear Localization Signal | NT | artificial |
| 33 | Nuclear Localization Signal | NT | artificial |
| 34 | Nuclear Localization Signal | NT | artificial |
| 35 | Nuclear Localization Signal | NT | artificial |
| 36 | Nuclear Localization Signal | NT | artificial |
| 37 | Nuclear Localization Signal | NT | artificial |
| 38 | PolyA | NT | artificial |
| 39 | PolyA | NT | artificial |
| 40 | 5′ UTR | NT | artificial |
| 41 | 3′ UTR | NT | artificial |
| 42 | Psi Element | NT | artificial |
| 43 | Spleen focus-forming virus promoter (SFFV) | NT | artificial |
| 44 | Spleen focus-forming virus promoter (SFFV) | NT | artificial |
| 45 | hPGK promoter | NT | artificial |
| 46 | EF-1a Core Promoter | NT | artificial |
| 47 | EF-1a Core Promoter | NT | artificial |
| 48 | U6 Promoter | NT | artificial |
| 49 | Primer Targeting KRAB (forward) | NT | artificial |
| 50 | Primer Targeting KRAB (reverse) | NT | artificial |
| 51 | Primer Targeting KCNH2 (forward) | NT | artificial |
| 52 | Primer Targeting KCNH2 (reverse) | NT | artificial |
| 53 | Primer Targeting KCNJ2 (forward) | NT | artificial |
| 54 | Primer Targeting KCNJ2 (reverse) | NT | artificial |
| 55 | Primer Targeting GJA1 (forward) | NT | artificial |
| 56 | Primer Targeting GJA1 (reverse) | NT | artificial |
| 57 | Primer Targeting GAPDH (forward) | NT | artificial |
| 58 | Primer Targeting GAPDH (reverse) | NT | artificial |
| 59 | Primer Targeting KRAB (forward) | NT | artificial |
| 60 | Primer Targeting KRAB (reverse) | NT | artificial |
| 61 | Primer Targeting ZIM3 (forward) | NT | artificial |
| 62 | Primer Targeting ZIM3 (reverse) | NT | artificial |
| 63 | pHR-UCOE-SFFV-dCas9-mCherry-ZIM3-KRAB | NT | artificial |
| 64 | pAV[Exp]-mCherry-SFFV-dCas9/KRAB/MeCP2 | NT | artificial |
| 65 | p154473-attachment_w5DDpndhwpbDmnEJw54 | NT | artificial |

### I. EXAMPLES

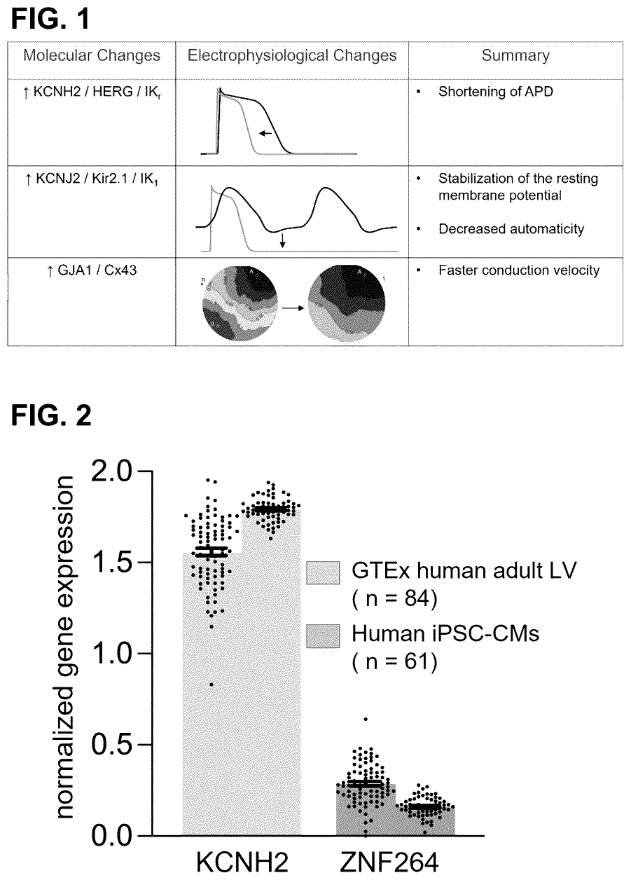

The development of new integrative approaches to address human cardiac disease, which faithfully can predict in vivo phenotypes from gene expression profiling and from therapeutic gene modulation is highly desirable. Three major ion channels implicated in cardiac electromechanical function were targeted: KCNH2, KCNJ2, and GJA1 and quantitatively linked multiparametric functional responses to protein levels and mRNA levels using an experimental pipeline (FIG. 1 and FIG. 2).

Induced pluripotent stem cell-derived cardiomyocytes (iPSC-CMs) have emerged as a powerful tool for disease modeling, though their immature nature currently limits translation into clinical practice. Maturation strategies increasingly pay attention to cardiac metabolism because of its pivotal role in cardiomyocyte development and function. Moreover, aberrances in cardiac metabolism are central to the pathogenesis of cardiac disease. Thus, proper modeling of human cardiac disease warrants careful characterization of the metabolic properties of iPSC-CMs.

Cardiomyocytes can be generated in vitro with high throughput and quality at a clinically relevant scale using pluripotent stem cells. However, these cardiomyocytes are phenotypically similar to ones at early fetal stage and often lack the attributes of adult or mature cardiomyocytes which are desirable for drug screening, modelling of adult-onset diseases, or replacing cells lost to disease. The major difference between immature and mature cardiomyocytes lies in the electrophysiology of the cells, which is determined by the presence or absence of specific ion channels on the cell membrane.

The experiments described herein show that Zim3, when used as Zim3-KRAB-dCas9 effector in interference CRISPR, without any guide RNAs, paradoxically upregulated key cardiac ion channel genes in human induced pluripotent stem-cell-derived cardiomyocytes (iPSC-CMs), which are responsible for healthy resting membrane potential, repolarization of the action potential and electrical transmission of signals. These yielded unexpected functional enhancements indicative of a more mature iPSC-CM phenotype.

### Example 1

Paradoxical Effects of Zim3 on Human iPSC-CM Electrophysiology

Zinc Finger imprinted 3 (Zim3 or ZNF657/264) is a protein-coding gene in humans, found at low levels in testis, brain, skeletal muscle and skin (Kim J, et al. (2001) Genomics 77 (1-2): 91-98). As other ZNF-KRAB (Krueppel associated box) domains, which bind to DNA and through complexing with KAP1 (KRAB-associated protein 1) affect chromatin organization (Stoll G A, et al. (2022) EMBO J. 41 (24): e111179), Zim3-KRAB may be involved in the regulation of transcription at multiple genomic loci.

In the human heart, the expression of Zim3/ZNF657/ZNF264 has not been examined in detail. RNAseq data in human adult heart (left ventricle, LV) was analyzed from the Gene Tissue Expression (GTEx) database (GTEx Consortium. (2015) Science. 348 (6235): 648-660 and in human iPSC-CMs (FIG. 2)

The Zim3 gene identifier was only expressed in a very small fraction of the samples, at negligible levels, both in the LV and in iPSC-CMs. ZNF657 was not expressed in either group of samples. The ZNF264 gene was most consistently expressed—in all adult heart samples and in all iPSC-CM samples, but also at very low levels. For comparison, the gene expression for KCNH2 (encoding for the main repolarizing K+ channel in cardiomyocytes) was also shown.

To quantitatively compare the two datasets, the transcripts per million (TPM) were normalize by the respective geometric mean (GM) across the transcriptome in each sample and displayed log 10 of the ratios (Pressler M P, et al. (2022) Front Cardiovasc Med. 9:941890). The adult human heart (LV) has a higher ZNF264 expression than the human iPSC-CMs, however both have very low expression overall.

Recently, Zim3-KRAB has been identified as a potent effector (repressor) in combination with dCas9 for interference CRISPR (CRISPRi) gene regulation studies (Alerasool N, et al. (2020) Nat Methods. 17 (11): 1093-1096. Indeed, using Zim3-KRAB-dCas9 adenoviral delivery in combination with suitable gRNAs in post-differentiated induced pluripotent stem cell-derived cardiomyocytes (iPSC-CMs), robust suppression of the expression of KCNH2 was shown with concomitant functional consequence (Han J L, et al. (2023) Commun Biol. 6 (1): 1236.).

Here, unexpected effects of simply expressing Zim3-KRAB-dCas9 alone on cardiac electrophysiology were generated without the addition of any gRNAs or with the use of scrambled/control gRNA.

Moreover, Zim3-KRAB-dCas9 expression significantly upregulated the mRNA expression of 3 key cardiac ion channels. First, KCNJ2, encoding for the Kir2.1 protein-inward rectifier K+ channel, contributing to the maintenance of healthy negative resting membrane potential. Second, KCNH2, encoding for the rapid delayed rectifier that controls action potential duration, APD. Third, GJA1, encoding for Cx43—the main gap-junctional protein in ventricular cardiomyocytes; for Cx43 this was also confirmed at the protein level.

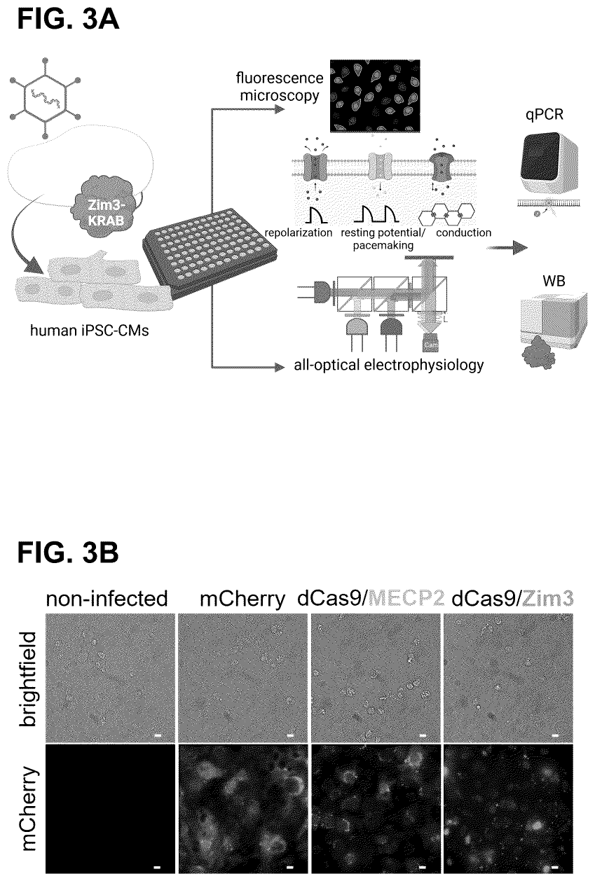

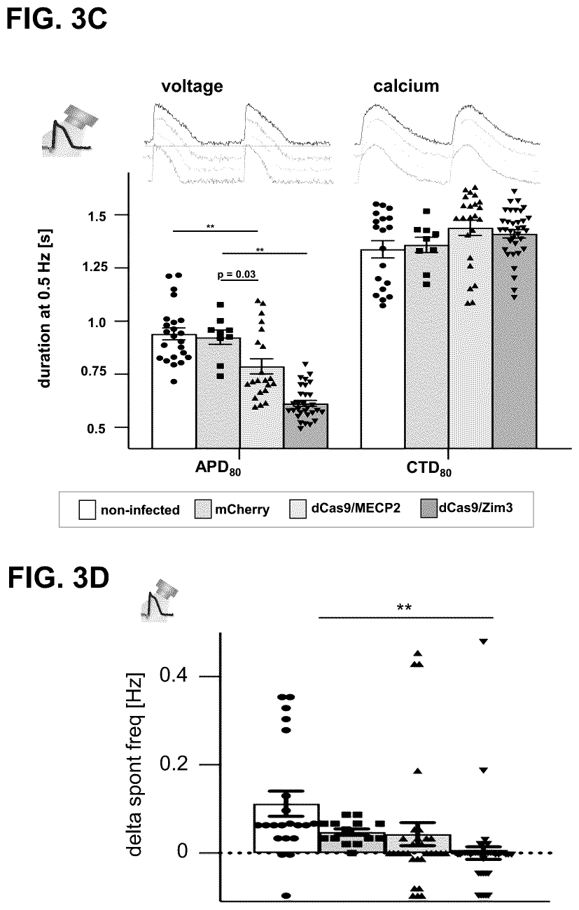

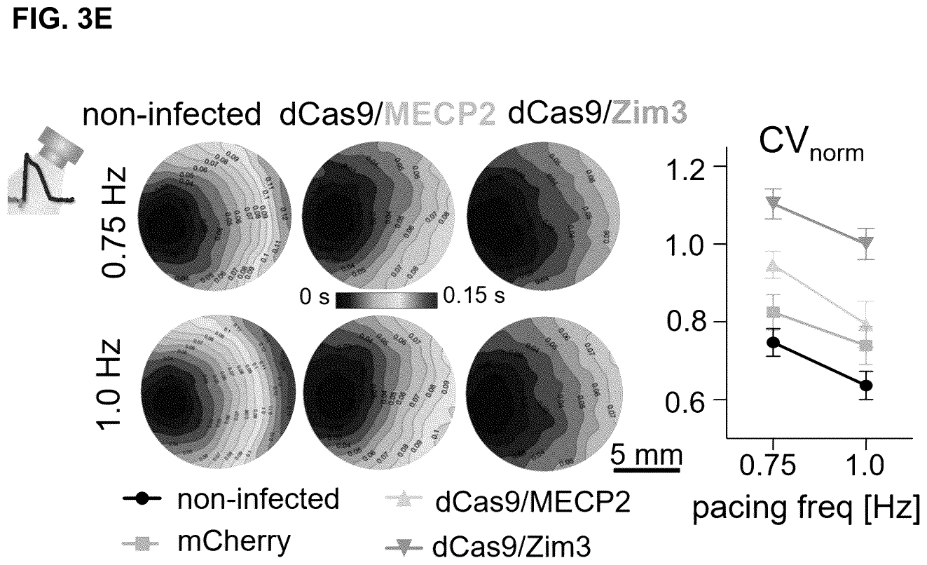

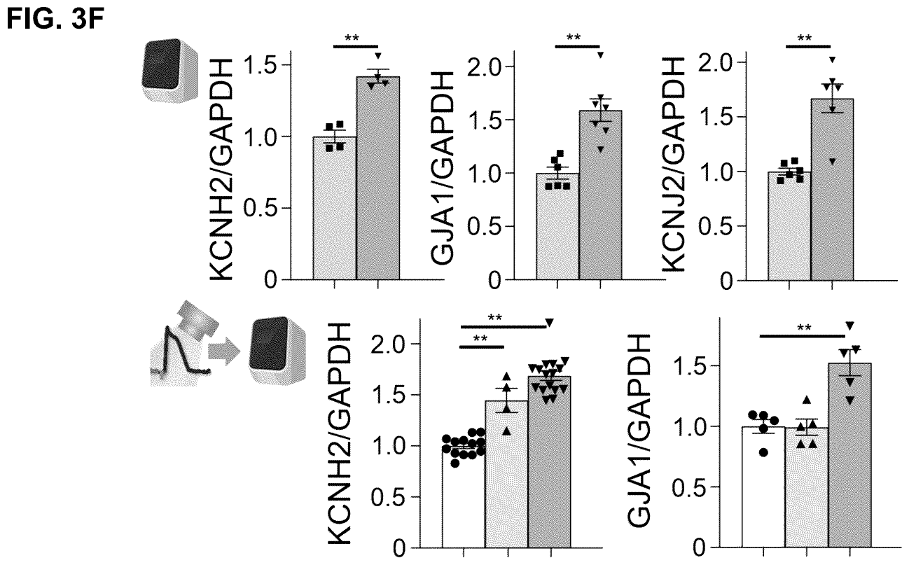

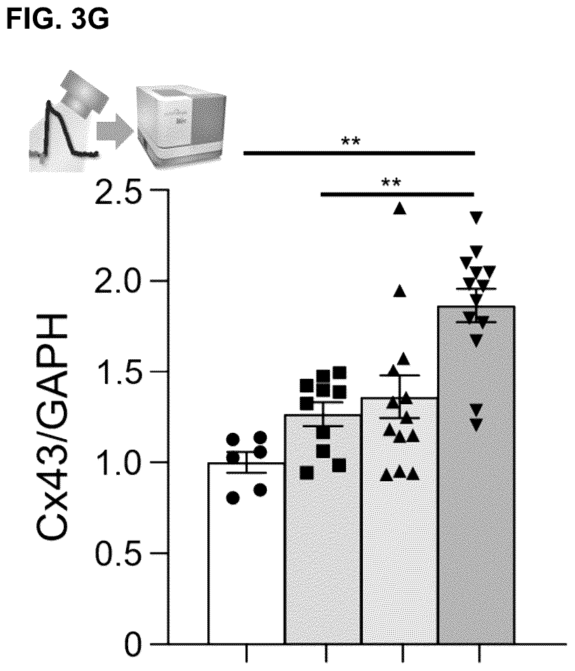

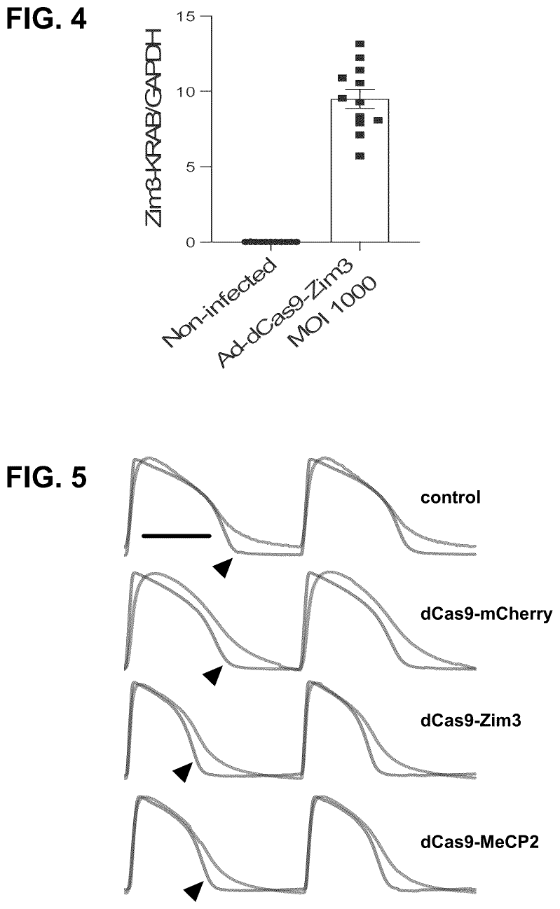

Using all-optical electrophysiology (as set forth in FIG. 3A), physiologically relevant functional effects, expected for (i) upregulated KCNH2 (APD shortening in paced conditions) (FIG. 3C, FIG. 3F); (ii) upregulated KCNJ2 (significantly reduced spontaneous firing), (FIG. 3D, FIG. 3F); and (iii) upregulated GJA1 (conduction velocity increase), (FIG. 3E-FIG. 3G) were corroborated. In combination, these electrophysiological changes are perceived as desirable and a signature of more mature iPSC-CMs.

These gene expression changes and functional effects cannot be attributed to the expression of dCas9 as regular KRAB-dCas9 (using the KRAB domain of KOX1/ZNF10) without gRNAs does not alter these genes (Han J L, et al. (2023) Commun Biol. 6 (1): 1236). The fluorescent reporter, mCherry, also does not have such effects.

Included here earlier-generation effector construct for CRISPRi-MeCP2-KRAB-dCas9 (a non-KRAB effector, MeCP2, combined with KOX1/ZNF10) (Yeo N C, et al. (2018) Nat Methods 15 (8): 611-616) caused similar, but much milder changes when delivered in the same type of viral vector-adenovirus with an SFFV (spleen focus-forming virus) promoter and an mCherry reporter, at the same multiplicity of infection, MOI 1000, as Zim3-KRAB-dCas9.

Interestingly, MeCP2 has been shown previously to act not only as a transcriptional repressor but also as an activator for many genes (Chahrour M, et al. (2008) Science. 320 (5880): 1224-1229).

Primers were designed to specifically target the KRAB domain of Zim3 to detect the overexpression of dCas9-KRAB-Zim3 in human iPSC-CMs using qPCR.

FIG. 5 shows optically paced action potentials (red—also indicated with arrow heads) and calcium transients (green) at 0.75 Hz pacing show the shortening of APD (and CTD to some degree) for dCas9-Zim3 compared to control and mCherry virally transduced samples. Note that dCas9-MeCP2 also shortened APD but to a lesser degree than Zim3.

The standard CRISPRi system (dCas9-KRAB-KOX1) was used in CRISPRi experiments post-differentiated human iPSC-cardiomyocytes (Gilbert L A, et al. (2023) Cell. 154 (2): 442-451; Han J L, et al. (2023) Commun Biol. 6 (1): 1236).

These experiments with that CRISPRi tool did not uncover effects on electrophysiology. Specifically, dCas9-Zim3 mediated CRISPRi yielded better results than the original system.

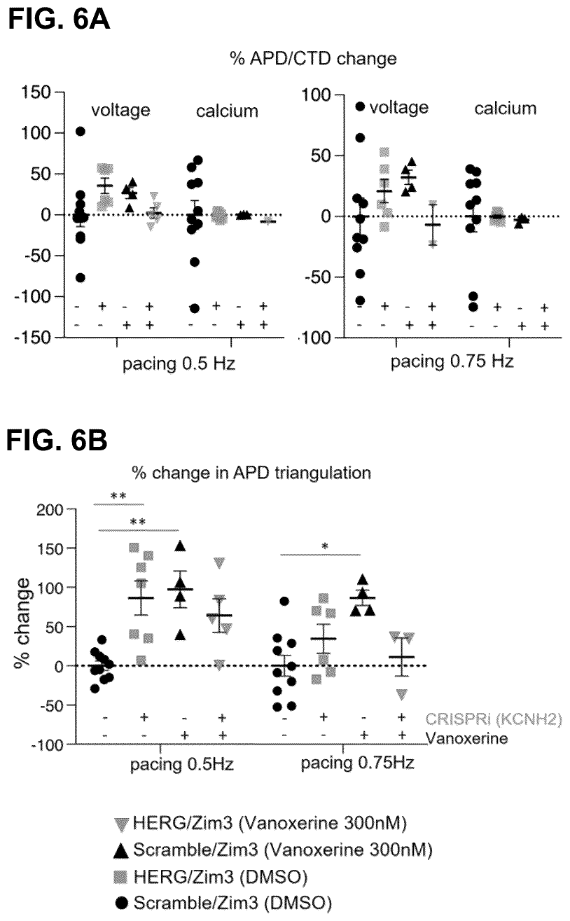

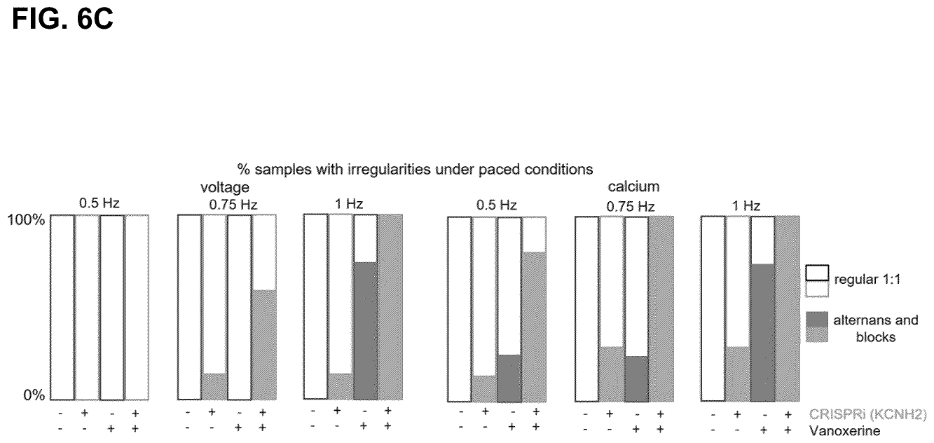

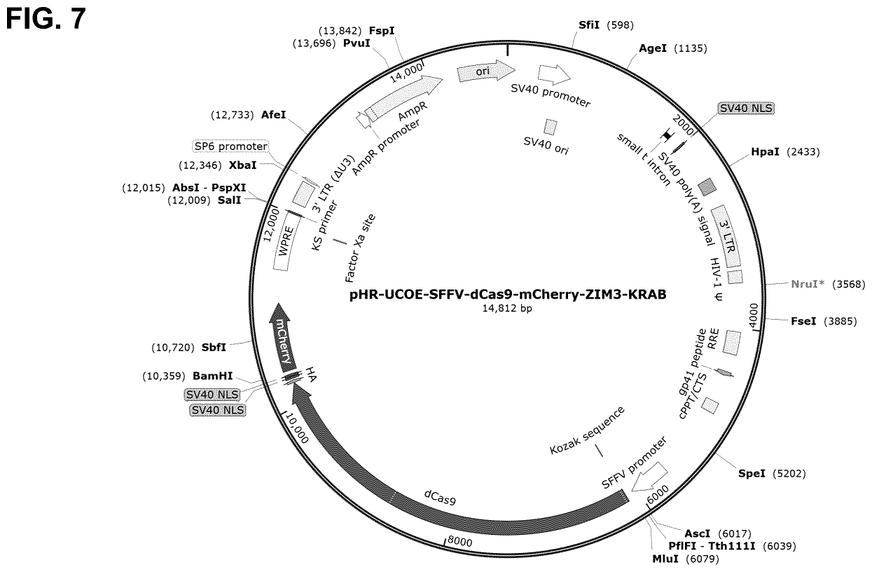

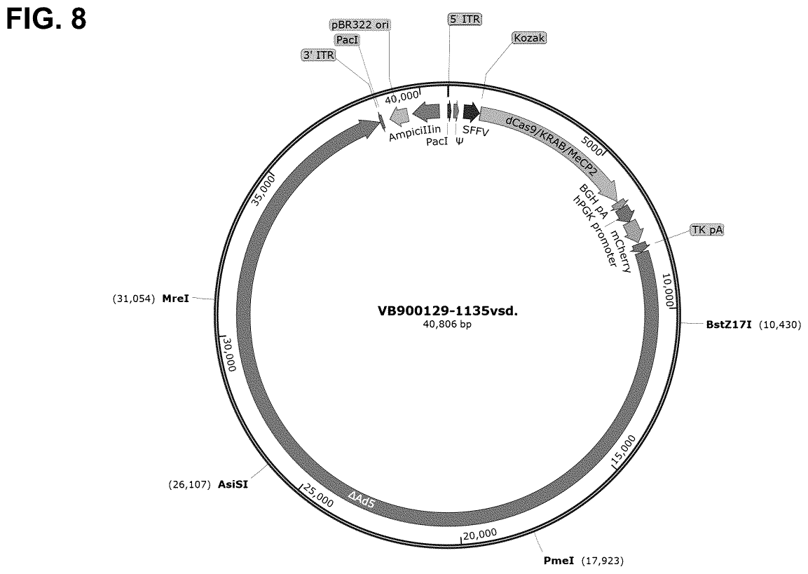

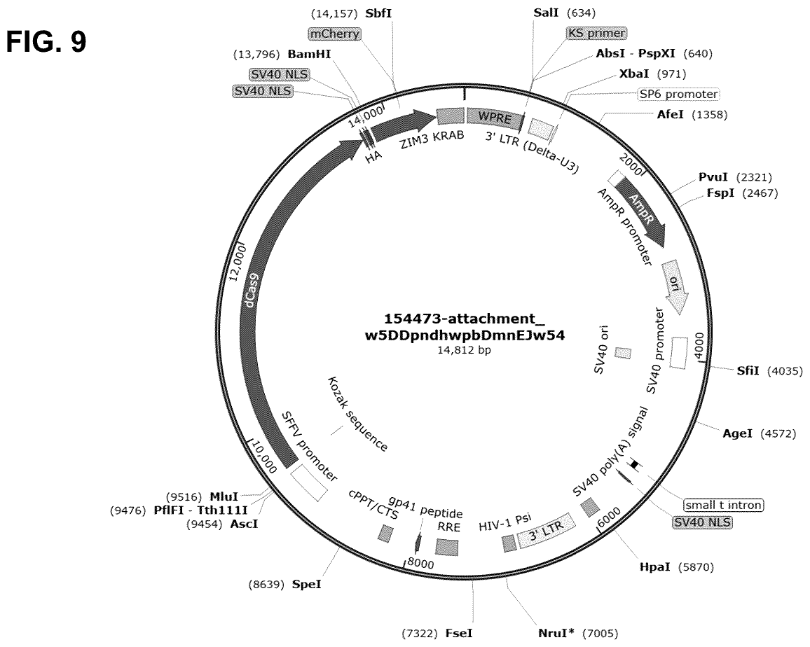

Another study was implemented to test CRISPRi without and with application of a pro-arrhythmic drug-vanoxerine. The first group below represents the application of dCas9-Zim3 with scrambled gRNA. In FIG. 6C, this group showed no irregular rhythms (alternans or blocks) in response to pacing at 0.5 Hz, 0.75 Hz, and 1 Hz compared to the other three groups which had vanoxerine applied and/or CRISPRi with gRNA for HERG.

### Materials and Methods

hiPSC-CMs Culture and Gene Modulation. iCell Cardiomyocytes 2 from a Caucasian female donor (Cat. C1016, Donor 01434, Fujifilm/Cellular Dynamics) were plated onto fibronectin (Cat. 356009, Corning) coated (50 μg/mL) glass-bottom 96-well plates (Cat. P96-1-N, Cellvis) at 1.56×10⁵ hiPSC-CMs cells/cm² and maintained following the manufacturer's protocol. For macroscopic optical mapping to quantify conduction velocity, hiPSC-CMs were plated onto 35 mm dishes with a 14 mm glass-bottom well at the same seeding density. Cells were cultured at 37° C. and 5% CO₂. hiPSC-CMs were infected with Ad-mCherry (Cat. 1767, Vector Biolabs) at MOI 50, Ad-dCas9-mCherry-Zim3-KRAB (Vectorbuilder), based on plasmid pHR-UCOE-SFFV-dCas9-mCherry-Zim3-KRAB, a gift from Mikko Taipale (Alerasool N, et al. (2020) Nat Methods. 17 (11): 1093-1096) (Cat. 154473, Addgene) at MOI 1000, and Ad-dCas9-mCherry-KRAB-MECP2 (Vectorbuilder) at MOI 1000 on Day 4 post-plating.

To confirm expression of the used genetic constructs, the fluorescence reporter mCherry was visualized using an inverted Nikon TiE microscope. More detailed methods have been described in Han J L, et al. (2023) Commun Biol. 6 (1): 1236.

All-Optical Electrophysiology Functional Experiments. For all-optical electrophysiology experiments, hiPSC-CMs were then infected on Day 6 with Ad-CMV-hChR2(H134R)-eYFP (Vector Biolabs) and functional experiments were conducted on Day 8. Both macroscopic and microscopic functional assays were conducted in Tyrode's solution (in mM): 135 mM NaCl; 1 mM MgCl₂; 5.4 mM KCl; 1.8 mM CaCl₂); 0.33 mM NaH₂ PO₄; 5.1 mM glucose; and HEPES; adjusted to pH 7.4 with NaOH at 30° C.

hiPSC-CMs were labeled with spectrally compatible fluorescent indicators for membrane voltage BeRST1 at 1 μM (from Evan W. Miller, University of California, Berkeley) and Rhod-4AM at 10 μM (AAT Bioquest, Sunnyvale, C A). For all-optical electrophysiology with an in house-built high throughput (HT) plate imager (Heinson Y W, et al. (2023) J Mol Cell Cardiol Plus. 6:100054), optogenetically (470 nm) paced and captured epi-illumination based imaging of fluorescence signals of voltage (Vm) using near-infrared dye BeRST1 with fluorescence excitation/emission at 660 nm and 700 nm long pass, respectively and [Ca²⁺]i using Rhod-4 with fluorescence excitation/emission at 530 nm and 605 nm, respectively using the Basler acA720-520 μm (Basler A G, Germany) at 100 frames per second (fps).

TTL-programmable LEDs were utilized for optical actuation (10 ms pulses) with a blue LED (SOLIS-470C, Thorlabs) at 470 nm. For macroscopic studies to quantify conduction velocity, the imaging system was used as previously described (Liu W, et al. (2023) ACS Photonics. 10 (4): 1070-1083; Heinson Y W, et al. (2023) J Biomed Opt. 28 (1): 016001).

The system is equipped for multiparametric all-optical imaging of voltage, calcium, and dye-free imaging of mechanical waves by oblique transillumination. Signals were filtered and analyzed using an automated custom software in Matlab to quantify relevant parameters, including spontaneous pacing rates, action potential duration at 80% repolarization (APD80), calcium transient duration at 80% (CTD80), conduction velocity (CV) (Liu W, et al. (2023) ACS Photonics. 10 (4): 1070-1083; Heinson Y W, et al. (2023) J Biomed Opt. 28 (1): 016001).

For some functional parameters, data were normalized with respect to the mean of the Ad-dCas9-mCherry-Zim3-KRAB group for each run/experiment. This applied to spontaneous rate comparisons (the group mean for Ad-dCas9-mCherry-Zim3-KRAB for each run was subtracted from the actual spontaneous frequencies observed in each sample. For conduction velocity, for all samples, the CV was divided by the group mean CV for Ad-dCas9-mCherry-Zim3-KRAB for each run.

Molecular Analysis qPCR and Western Blots. To quantify the effects of the virally-delivered constructs on key genes of interests, qPCR quantification was performed using POWER SYBR™ Cells-To-CT Kit (Cat. 4402955, ThermoFisher Scientific) and protein quantification was performed by capillary electrophoresis (Wes™ by ProteinSimple) in lysed samples from the 96-well plates following the settings, primers and antibodies detailed in Li W, et al. (2022) Methods Mol Biol. 2485:15-37.

The qPCR analysis was applied either on pristine samples (prior to all-optical electrophysiology) or as part of a pipeline of characterization of the same samples, following functional optical measurements. Forward and reverse primers are set forth in Table 1.

Protein analysis of Cx43 was applied always after functional measurements, using a primary antibody for Cx43 ab11370 (Abcam) and for a reference protein GAPDH ab181602 (Abcam).

RNA Sequencing. For analysis of the gene expression of Zim3/ZNF657/264, the Genotype-Tissue Expression dataset (GTEx Consortium. (2020) Science. 369 (6509): 1318-1330; GTEx Consortium. (2015) Science. 348 (6235): 648-660), GTEx version 8 was used. RNAseq data was generated from the left ventricle (LV) of 84 female hearts (aged 20 years old to 70 years old). The transcripts per million (TPMs) were normalized by the geometric mean of the whole transcriptome for each sample and the log 10 of the ratio was used as described in Pressler M P, et al. (2022) Front Cardiovasc Med. 9:941890.

Human iPSC-CMs for a Caucasian female donor were grown in dense syncytia at 150,000 cells per well in 48-well format plates. On day 6 after plating, the cells were washing in cold PBS, then lysed in 50 mL Trizol per well and flash-frozen in RNAase free cryotubes. The RNA sequencing was performed by Genewiz using Illumina HiSeq 2×150 bp sequencing. The obtained TPMs were normalized by the GM, as the GTEx data.

Statistics. All data points are shown in the graphs plotted with GraphPad Prism V8. Statistical analysis to compare groups included one-way ANOVA with post-hoc Tukey correction conducted in Prism.

### SUMMARY OF EXPERIMENTS

As described herein, expression of Zim3-KRAB-dCas9 significantly upregulated the transcription of several key cardiac ion channels in human post differentiated pluripotent induced stem cell derived cardiomyocytes (iPSC-CMs). These channels include KCNJ2 (Kir2.1), KCNH2 (HERG). and GJA1 (Cx43, which is the main gap junction protein in ventricular cardiomyocytes). The combination of these effects produced desirable electrophysiological changes in line with more mature iPSC-CMs with anti-arrhythmic properties.

What is claimed is:

1. A method of improving or enhancing maturation of iPSC-CMs, the method comprising:
contacting one or more iPSC-CMs with (1) a nucleic acid molecule comprising a nucleic acid sequence encoding (i) at least one zinc finger protein, (ii) at least one polypeptide having effector activity, and (iii) a deactivated Cas9 (dCas9) endonuclease, or (2) a vector comprising the nucleic acid molecule,
wherein, following the contacting step, the one or more iPSC-CMs are characterized by a mature structural, electrophysiological, contractile, and metabolic profile.

2. The method of claim 1, wherein the nucleic acid molecule encodes a Zim3-KRAB-dCas9 effector.

3. The method of claim 2, wherein the encoded Zim3-KRAB comprises the sequence set forth in SEQ ID NO:23, and wherein the encoded dCas9 comprises the sequence set forth in SEQ ID NO:20.

4. The method of claim 1, wherein the nucleic acid molecule does not comprise the sequence of a guide RNA.

5. The method of claim 1, further comprising characterizing the maturity of the one or more iPSC-CMs.

6. The method of claim 5, wherein matured iPSC-CMs are characterized by mitochondrial maturation, increased oxidative capacity, and enhanced fatty acid use for energy production.

7. The method of claim 5, wherein matured iPSC-CMs are characterized by an increase in the expression level of KCNJ2, KCNH2, GJA1, or any combination thereof.

8. The method of claim 5, wherein matured iPSC-CMs are characterized by an increase in the expression level of PDK4, CD36, PPARA, ATP5, LPL, SCD, PPARD, ACADVL, ACAT1, DGAT1, PPARGC1A, ESRRA, N2B, CAV3, SERCA2, CPT1A/1B, or any combination thereof.

9. The method of claim 5, wherein matured iPSC-CMs are characterized by a decrease in the expression level of ALDOA, HK1, HK2, PGK1, GAPDH, LDHA, or any combination thereof.

10. The method of claim 5, wherein matured iPSC-CMs are characterized by an increase in the number of mitochondria and mitochondrial size, an increase in the number of peri-sarcomeric mitochondria, a decrease in the number of perinuclear mitochondria, or any combination thereof.

11. The method of claim 5, wherein matured iPSC-CMs are characterized by an increase in the expression of genes encoding electronic transport chain (ETC) proteins.

12. The method of claim 5, wherein matured iPSC-CMs are characterized by a reduction in glucose uptake, glycogen storage, lactate production, hexokinase activity, the proportion of glycolysis-related ATP production, or any combination thereof.

13. The method of claim 5, wherein matured iPSC-CMs are characterized by beating when stimulated with a force from about 40 mN/mm² to about 80 mN/mm².

14. The method of claim 5, wherein matured iPSC-CMs are characterized by a conduction velocity of from 55 cm/s to about 65 cm/s (e.g., indicative of mature CMs).

15. The method of claim 5, wherein matured iPSC-CMs are characterized by beating when stimulated with an upstroke velocity of from about 150 V/s to about 350 V/s.

16. The method of claim 5, further comprising adding matured iPSC-CMs to an implantable cardiac patch.

17. The method of claim 5, further comprising using matured iPSC-CMs to assess cardiac toxicity and arrhythmogenicity of one or more developmental drugs.

18. An implantable cardiac patch, comprising: iPSC-CMs matured by the method of claim 1.

19. A method of slowing and/or preventing progression of a cardiac disease or disorder in a subject, the method comprising:
implanting in a subject having a damaged or diseased heart the implantable cardiac patch of claim 18, thereby reducing the pathological phenotype associated with the cardiac disease or disorder.

20. The method of claim 19, wherein reducing the pathological phenotype associated with the cardiac disease or disorder comprise restoring one or more aspects of cellular homeostasis and/or cellular functionality and/or metabolic dysregulation.


SEQUENCE LISTING (65 sequences)

SEQ ID NO: 1 - protein, 472 aa, Homo sapiens
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKETLTTQKGVECDGSKKILPLGIDDVSSLQHYVQNNSHDDNGYRKLVGNNPSKFVGQQLKCNACRKLFSSKSRLQSHLRRHACQKPFECHSCGRAFGEKWKLDKHQKTHAEERPYKCENCGNAYKQKSNLFQHQKMHTKEKPYQCKTCGKAFSWKSSCINHEKIHNAKKSYQCNECEKSFRQNSTLIQHKKVHTGQKPFQCTDCGKAFIYKSDLVKHQRIHTGEKPYKCSICEKAFSQKSNVIDHEKIHTGKRAYECDLCGNTFIQKKNLIQHKKIHTGEKPYECNRCGKAFFQKSNLHSHQKTHSGERTYRCSECGKTFIRKLNLSLHKKTHTGQKPYGCSECGKAFADRSYLVRHQKRIHSR

SEQ ID NO: 2 - RNA, 2627 nt, Homo sapiens
  source 1..2627: mol_type = mRNA
ttccattggaaactgattgggcagatttgaaaattaagctttcacaatcacaatttttatgagagttcattcagttttggagaagtcgaggaaagaattgggaattgaaattttgatgtggcctgataggatcaggtggggcttttgataggaagtgagtgttttgggttgtttcttgactggattatgatgcggaatacagcgggtagagggccgattaagttttagtcattagtgtagcttttcagtatacacgccttttcattttcagtccctttcagaagaaaagaccacaggcatcagaaatcaatcaacttgctaatttatgccagattccactggagatctgccttcccaagactttactgactggtgaagaacaggaaatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctcgcaagagaagtcccatcaatcaataaggaaacgctgactacgcagaaaggtgtagaatgtgacggatctaagaaaatacttccactgggcatagatgatgtatcttccttgcaacactatgtacaaaataattctcacgatgataatggatacagaaaattagttggcaataatccatccaaatttgtaggacaacaactgaaatgtaatgcctgtagaaagttattcagttcaaagtcacgccttcaaagtcacctgaggaggcatgcctgtcaaaaaccctttgaatgtcatagctgtggaagagcattcggggagaagtggaagcttgataaacatcagaaaactcacgcagaggaaaggccctataaatgtgagaactgtggaaatgcctacaagcagaagtcaaatctctttcaacatcagaaaatgcatactaaagagaaaccctatcagtgtaagacatgtggaaaagccttttcctggaaatcatcctgcattaatcatgagaaaattcataatgccaagaaatcctatcagtgtaatgaatgtgagaaatccttcaggcagaactcaaccctcattcaacataaaaaagttcacactggacaaaaaccctttcaatgtacggactgtggaaaggctttcatttacaagtcagatcttgtgaaacaccagagaatacacacgggagagaaaccctataaatgtagcatatgtgagaaggccttttcccagaaatccaatgtcatcgatcatgagaaaattcacactgggaagagagcttatgagtgtgatctatgtggaaatacctttatccagaagaaaaacctcattcaacataaaaaaatccatactggggaaaagccctatgaatgtaacagatgtggaaaagccttctttcagaagtcaaaccttcatagccatcagaaaactcatagcggagagaggacctatagatgtagtgaatgtggaaaaaccttcatccggaaattaaaccttagtttgcataaaaaaacccatactggacaaaaaccttatggatgttctgaatgcggtaaagccttcgctgacaggtcataccttgttaggcaccagaaaagaattcactccagataggagccccatgcacttaaaaaaacatgggaagatatggttgcctggaattgtcaccccagaattccatgttgaaatggcctctaatttggaggctatttttgaaggtagcgaggacttattatcacagtatcctttgaaaacccattcggcatactcactcagcaaatgtttattaagtataaatatttattagatgccttttgtgtaccaggaactcttaaacactgaagatgtagcaataaaccaaatgtgctaaaaccctgatggagttttattccagtgaataatgaccagtggtatccatatctttaaaaacgtttaaaagactatacttttggccaggcgtggtggctcaaatgcctgtaatcccagtactttgggaggctgaggtgggcgcatcacctgaggtcaggagttcgagaccagcatgaccaacatggtgaaaccctgtctctaccaaaaatacaaaaattagccaggcatagttgtgggcacctgtaatcccagctacccgggaggctgaggcaggagaattgcttgaacctgggaggtagaggttgcagtgagccaatattgcaccattgcactcctgcctgggcaacaagagcaaaatttcatctcaaaaataagataaaataaaataaacaaataagacagcactttatacttttagggatgatttctataattcatgagtggagaggtttctctagaggtgtaggggactggaaaccacatccaggaagtacagctttctctcctgagcatgaaggtatcttggcatttcttcactgcaattgccatttgccttttttgtttttc

SEQ ID NO: 3 - DNA, 11022 bp, Homo sapiens
  source 1..11022: mol_type = other DNA
gcaggggagtaggataggaatccccgccgcacctttgtacgagcctgaccccttccgtgggtttgttcctgggtcgccgtcaagctgcggtctctcctcccccgcccttcagccccgcggtctccaggggcggcgccctgggtctggaacgcggttgccaccgaggaggcggcggccctgcgtctggaacgccgttgccaccgaggaggcggcggccccgagcgcgcctggaagccccgggcaaccggccagggtcgggcacaggtggggtccgtcaggccgcccggggctcctctgtcccagctctgcggcccagggggtgacgtgatggcggcagcggtgctgacggaccgggcccaggtgagtggacggtggcttcgcggttgccgcttccttgccagcgctgaggcggtttccgaagtgggtgggagcccaggtatcccctggatttgtcttcgcagtcgtcgttcgctttggtgtgtggagctggtttgccacttaatttatacctggccgtaaggagtgttgggcctactggaaggagctccgtaggttgttgagtgaatgatgcttcctctggggactcccatcatccttggcctgcaagcattctctggtggaccaggtctcattccagcatgaattgggggcctgaggggggaaggggagggcagggcagtagcaagccgacaaatgcgcgagcagagacaaggtatgttgtatgtttaaggaggctcaggagggtgtgtaaggttgccatggttttcctgcggccgctgagacgtggagggcaacagtgtgtggtcttttcctggatgttctagccatccgcaactactgcagacattgtggccagtcagatgcccttactctcatcgctacagcctgcattatccttggccgctcgtgttccgtggccccaaagtatcctggcgtcagagacaagttctgtcggaacccagtaaggaaagaggccgatggttggttcattgtggaatttccttgggggatctgggagacccagggagatcccagagggcatgggaagctggtctggagcctgtcgttccccaagtagatgcagcccttccagtgccgctctgtggggtcagtgaggaatgctgagctttttgtgcccgtgttcacatacttttttttgttttgagacagggtctcactctgtcgcccaggctggagtacagtggtgcactctcagctcactgcagtctctgcctcccaggttcaagcaattctcctgccccagcctgccgagtagttgggattacaggcatgcactaccacacctgactgatttttgtattttagtagagactgggtttcaccatatttgccaggctggtcttcaactcctggcctcaagcgatccgcccacctcgacctcccaaagtgctgggattacatgcatgagccaccatgcccggcccacagatacgtttttcagggctaattgaaatagacttaatgttagaaatgagtggaaatgcttataacttcatcgcattagatttgctttgatgttgggggagtgttggagttacactgttagaccgtgaatcatagtaaccccatgtttagatcctatctatggtggtgagatgtgtgactgggtgaattgatagcgcttccttagcatgaagtcattgactgtgaaggtgatgatggtgttgactgcagggactgtgaaaggattaataaaaatcccagagcacactgagcattgaatactttaaatttaacaactgttactggtgggaagtgttattgtattgatccagaaatcaaggctcagagaaatgaaatgctgtctttgaggtaagagctgtcaagtgtcagtttcaagtgtttggagctaggaacttctagtcactgtgtttacacaggatgtgaagccttcaacatggggcaggcacctagctggaccagtgagactcaggtactgccgttgtaaccattattgctattattaccatttccccatttccctgtcgttttagagcccttagcttctgtatttcacagtccacaagttgtttccgttgtcctgtgtcattctctaacggccgttaaataggaggcttctctcctttactctggtattatgttaaaagcatctatcttccctcttgagcccagcacagagctaggccctcaggaggtgctctgtgattcaggccgccatcactgtacaccttttccagtggagcacagtctgtgatctcattcagcagctgaaaggccaatactactaattctgtttgactttccaggtgtctgtgacctttgatgatgtggctgtgactttcaccaaggaggagtgggggcagctggacctagctcagcggaccctgtaccaggaggtgatgctggaaaactgtgggctcctggtgtctctgggtaaggccttccttccccctccaccatgcaggtgacctgccacttccttcattccatcttctgactccaggcctggctgcacaagaaggcctctagaatctaccttgcctctttcccacagctttagtcattttccacagtaacctcttcctatctggtctcctatatctctgggctaaagacagggtactctttggctcccagccaaaggagaaggtggtaaaaggaaatggggtgttttgcaaactcaaaggtaattttcagctgtgcctcatgaggggctggtgtaggaactgagaaccaccacgatggtgtcctggcttctctgtctgggtctgctcatgtcacgtctattctctatggctttttctgccttagtgagttcaagacaagagtggccacctggattagggttctctaaagggacagaactaatagaatagatatatataaaaaggacagtttattaagtagtaataactcatgattacaaggtcccacagtaggctgtctgcaagctgaggagcaaggaagccagtccaagtcccaaagctgaagaacttggtgtccaatgttcgagggcaggaagcatccagcatgggagaaagatgtaggctggtagtctaggccagtctagtcttttcatgtatttctgcctactttatatgctggcctcgctggcagctgattagatggtgcccacccagattaaggggggggtctgcctttcccagcccactgactcaaatgttgatctcttttggcagcacctttacagacacacccaggatcaatacgttgcatccttcaatccaatcaagttgacacattaataaaaatcccagagcacactgagcattgaatactttctctttaaatttaacgactgttactggtgggaagtattattgtattgatccagaaataaaggctcagagaaatgaaatgatgtttttgaggtaacagctgtcaagtgtccgtttcaagtgtttggagctagggacttctggtgactgtgttaacacaggatgggaagccttcaacatgggccaggcacctagctggacctagctgttaaccatcacaactccatcccttgtcaacttgaacccatacacatctcctgagatcatacataatcttcaaataaagacaataaggtcataattatgcttaacataatacaagtatcgttcatgcaaccgaaacacactaatccccaacccaagtgctattacataaaggtagcaatacttaaatgctgatatgaagtcagtaaatcttacgtcacatgataaaggaaataaaatgcagatgttttcttagtacaagtgtatacatcacaaacatgtttttaataaaagaaggaggaaatacaacaattacagtcctcatttctgcaactggtcacttggtcgtagttggtattaatgactaccttcttctcctgcacattctgtattcactttgccttcagcaagcacctcagcaggtcatggttttttttcctggtggagtgacccaaaccttcattcctgaagggtctgggccatttgtagtcctgcctggattgggctgttgtagcttcccattgaccttaatcacagggcatggtaatactaagagatgccctaatggatcttctgtattccatgcatactcttccttacctccattgtggagtagtagactgatttcatcttgatagtctgggtcattcatcccagccaacactgtaactcccttcttaggctattgacttaaaggtaggaggagcccaaagtgtccaggtggcaatcttaacttccagtttaatggaatcgttgtgtctcctggtggcagcattcctccctctgaaactaagacttctaggccaacggaatgtaatgtcgcgggaataggaagcaaaattttgctagttgatcactagggatgatgttgagtggtgccatttgcacttccaccccttgattcctggacccatgaatcctggctatgggagaaacagtaccatatattggatgctaattcagagcatacatggcctactggagaactttgccccagccctgcaaagtattgtcactagttggagtaattatgactacaaaaggtcattccaccattctgtcaatccagttgcttcaggactatggggaacatggtaagatcagtgaattctgtgagcatgagcccactgctacacttctttagccataaagtgagtgccttggtcagaggcaatgctgtgtggaataccattacgatggataaagcattccatgagtccgtgaatggtagtcttggcagaagcactgcgtgcaggataagcaaacccatatccagagtaagtgtctattctagtgaggacagacctctgccctttccatgatggaagaggtccaatataatcaacctgccgccaggtagctggctgatcaccccgagggatggttccatattgagggctcagtgttggtctctgctgctggcagattgggcactcagcagtggctgtagccaggtcagccttggtaagtggaagtccatgttgctgagcccatgcgcaacctccatccctgccactatgaccattttgttcatgggcccattgcacgatgacaggggtggctggggaaagaggctgagtggtgtccacagaacaggtcatcctatacacttgattattaaaacctttctctgctgaggtcttccattggtgagcactcacatgggatacagatatcttcacagtttttgaccactcaggtccatccacatccctcttccccagatttctttgtcaccaactttccagtcatccttcttcgaagtccctgaccatccagccaaaccattggctacagcccatgaatcagtatataattacacatcttctcatttctccttccatggaaagtgcacagcgaggtgcactgctccaagttctgcccactgggaaatttcccttcgccactctccttcagggatgtcctagaaaggggctgtagtgctgcagctatccactttcaggtggttcctgcatatcatacagaaccatctgtgaaccaggccctagtcttctcttcctccgtcaaccgatcatagggaacttcccatgaggccatcggtgcaggctgggggagagaaggtggggtggcaggagtggagtctgtgggcatttgagccacttcctcatgtaacttgtgccttcaggatccgcttgagcccaatcacatatataccactttcatctgatgatggaatgctgctgtgcgtgacccacttgatggctagctgggtcagaaagcacccagttcatggtaggcagttcaggttgcatggtgatttgatgacccatagtgtaacaggccaagagctgtctctcaaaaggagagtagttatctgcagaagatggcagggccttgctccaaaatcctagaggccttcgctgtgattcacctatgggggcctgccaaaggctccaaacagcatccctatctgccactgacacctcaagcaccattggatctgctgggtcttaggggccaagtggcagagcagcttgcacagcagcctagacctgcctgttgcagagccttctcctgttctggatcccactcaaaactggcagccatttgggtcactcgataaatgggctggagtggcatacccaaatgaggaatgtgttgcctccaaaaaccaaataggcccactaggcattgtgcctctttcttgattgtaggagggaccaaatgcagcaacttactcttcaccttagaaggcatatcttgacaggccacacaccactggacccctagaaattttactgaggtagaaggtccctaaattttagtcagatttatttcccatcctctggcacacagatgtcttaccagtaagtccagtgtgtttgctacttcttgctactggatccagtcggcataatgtcataaatgtaatggaccaatgtgatatcttgtggaagcaaaaagcgatcaaggtctctctgaataagattacgacacaaagtcggagagttgataggcccctagggtaggaccgtgaaggtatattgctggccttgccagctgaaggcaaattgcttctggtgggccttatggacaggaatggagaaaaaggcatttgccaagtcagcggctgcataccaggtaccaggagatgtgttaatttgctcaagcaatgaaaccacatctggtatagcagctgcaattggagcaaccacctggttaagctttctataattcacagtcattctctaagatccgtctgtcttctgctcaggccagatgggagagttgaatggggatgtggtgggaatcaccacccctgcatcattcaagtccttgatggtggcactaatctccacagtccctccagggatgtgatactgtttttgatttactatttttctaggtagaggtggctctaatggcttccatttggcctttcccaccttaatagccttcaccctaacagggagccagtgtgggggttctgccagctgctaagtatgtctgtgcctattacgcattctggcactgggaaaatgaccacaggttgagtctggggacccactggacccactgtaagtcaggcctgagctaaaactcctgacctccataagcccctactttaactggaggaccacaatgatgttttgggtcccctggaatcagcgtcagctcggagccagtgtcctgtagtccccgaaatgtctgatcatttccctttccccagtgcacagttaccctggtaaaaagccagaggtctccttagggaaggatgagagaaagattaacagcataaattgtcagtagtgtagtggggtccttcctcaagggacctggcctccccttcattcaaggggttctgggtctgtaaactagcccaagtctggaaattgaagggccatgattctgtttttgtaattcaaattagtcttttgtccattcgacctggaagttttctgcttatataaattaaataggattgcagtaggcttcctatcagtttcatttctgggaacactgtgattaattagccaacgccagagatctacatgagtcagactattctgattgctgctttgtctctgttgtccattatgatagctatgcctaccttgcctttgatggttgagtgccaccacttggcccctgccacctcgggatccaattattcccaatgtattaaatttttgtaggtgagtgactgaaattccaactgctggatctggcaattacagggctcttcaaagatgcaggtgctgccctcgcaaatctattttgcaagccattggttaaggttatgtcttatggaccctcccagctgggatgagtaggtctaaagtgactaatctactccatcatcccaatctccctaagcctttggatcccttcctctacattagaccaagggagatcaagcattcccagctcacccatgatgggccatcttttaatccatatttcagctagccaagcaaataagctattggaaccttttttaactcctcaggctgcaacattaaatgcaggatccctaattagtgagcccaaatcaataaattcagcctgatgcatctgtatgttccttccagcattatcccacacccttaatatccattcccgtgcctgttctccaaatttctgcttatataaattagaaaactcaattcaagtgtagctcacctccccatgggtcacactctgaacctcacttctgtgggcccatcgggactttagttataggtctagaagcaaacaaaggtgttgagggtgctagaggagaatcgatattatcttgcctggcaactgcctcaggggaggccatcactgttgcctcaggcagcacagggtttatctcctcagacaaaggtggaaaggctgatggcagcatgggtcggggagaggatgttgccactattggggatgccattttcctttctctttcttctgcctattaaacctccactcctaaaaaaaaaaaaagaaaaaatgatgaactcagggattctaactcctggcttcagaagcagatactaagcctcaaatctgctaagattgccctgagtgagtcttatctcctgtagagaaaaagctgaaattgtagaaaaacagacataagctagtcatgtgagtggctgacctgcaacaaaaggtgcatgcacagcctcaccaggtgtctactgttaaagtgagggcactgattggaaaagaatgggaccctgaaacttggaatagggatgtgtgggagaaccctgatgaagctggggacactgagcttgtaaactctgatgaaccttttttgccatcccccaaatgccatcccagcgctttgggaggccaagatgggaggatcacttgagcctagaagtttgagacaaagcctgggcaacatagtgagaccctgtctctaaaataaattgaaaaaaaaaaaagccactcagtaccctgttttactcccccaccccaccccgacctttgggtcttgtctttttctttgtctttgtctcttgtctcttgtgtcttgtgtcttgtcttgtcttgtcttgtcttgtcttgtcttgtcttttctcttttccttttctttcgttttcttttcttttctttttctgtcttgctgtcttgcccaggttggaatacagtggcacaatctcggctcactgcctttgcctcctaggttcaagcaattctcatgcctcagccacttaagtagctgggactacaggtgtgtgccaccacacccagttcgtttttgtatttttattaaagacgggattttgccatgttggccaggctcgcctcgaactcctgagctgaagtgatccatccgcctcagcctcccaaagtgctgggattacaggcgtgagccaccacacccaccctttactttttataataaaattttttttatacatttgttatatatgtgctatatataattaccatatatgatttgttataaacattagtatataaatgctggatttctaattggatcaaattatggacattgttttaatttacttattgtatgtgtatatatatacaacttatttatgtgaagatatttatatatatagtttttaagactaaataagggtggtattgtgtttgctaggcctggcatttgtatggtacataaggtttaaaagttcactttcttttttctgccccttgggtttgtagctgcctttttctttttttcgttatttctctctttgtgttttaggcttttggcatccagaagtagtggatcttagtttctgtccctgggtagtaaaaagaagctggggagaggaagaggtttgatgcctccaggcagctgcctctccctggtacagcctctgtcccaagtgtaatcagtcccccgctaaatgactagtctcttacaggatgggaagagagaataaataactcagttaagcagcccaccagtgactttggtgttggcttggttgattggacttgtatcccaacacaatccatcagaggaagtctctactcactaccttttgtgaggagcctcagccttgtatattaccccgtaaaccaacccagaaactaggtgtgtttatgttggaaaagtagactaaggagatcagtctgtcccagaacggtgccagctggggaattactgcccttgcgtatctagactgtggcatctttaaacactggaaggatctgaggagcactcctttaggattcctacagcactttaggccagaaacccatggcagcaaatgccagacgcagctgaagcacaaggctgccagcatctcgcagccttccccgggcagaactttctgtgtggtacaatcccttagtgagctgatcttcagccttcccatgagcgcagagtcagcctgctgggtgcaccagttgtattaatttgtatctatgggccaagtgtggtagctcatgcctgtagtcccagcagtatgggaggcaagagggttgcttgagcccaggagtttgagaccagcctgagcaataaagtgagacccagcctctacaaaaaaaatcaaaaaaattatctgggcgtggtggcacatgcctgtagtcccagctactcctaaggctgagatagggaagattgcctgagcctgggaggtagaggctgcagtgagccatgatcacaccactgcactccagcctgggcgacagagtgagaccctgtctcaaaaaaaagcaaatttgtacccatgggattttaatttatagcagtgacttagtgatgacacagagaggttaataacattgaaagaagaattcattatttacaattcgtgaaagaagagggcatatgctgggcctcatggggatacagctggaagcacaggggaggagaaagcctttaactgggtgtggtattctgatacatactggtttttgtccaccgttcctgattcataacccctatagcccttttgttaaaatgctgggtatgttaggcctcagggtcaggcctctgacctcctgccctcctttcgctctaacgttcctccacctttctgactgtgggtctcatgagtgggtccaaccctataccctgggaaaggaatgttgatgttttgaagct

SEQ ID NO: 4 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 5 - RNA, 1523 nt, Homo sapiens
  source 1..1523: mol_type = mRNA
atttttttggtgtgtgtgtcttcaccgaacattcaaaactgtttctccaaagcgttttgcaaaaactcagactgttttccaaagcagaagcactggagtccccagcagaagcgatgggcagtgtgcgaaccaaccgctacagcatcgtttcttcagaagaagacggtatgaagttggccaccatggcagttgcaaatggctttgggaacgggaagagtaaagtccacacccgacaacagtgcaggagccgctttgtgaagaaagatggccactgtaatgttcagttcatcaatgtgggtgagaaggggcaacggtacctcgcagacatcttcaccacgtgtgtggacattcgctggcggtggatgctggttattttctgcctggctttcgtcctgtcatggctgttttttggctgtgtgttttggttgatagctctgctccatggggacctggatgcatccaaagagggcaaagcttgtgtgtccgaggtcaacagcttcacggctgccttcctcttctccattgagacccagacaaccataggctatggtttcagatgtgtcacggatgaatgcccaattgctgttttcatggtggtgttccagtcaatcgtgggctgcatcatcgatgctttcatcattggcgcagtcatggccaagatggcaaagccaaagaagagaaacgagactcttgtcttcagtcacaatgccgtgattgccatgagagacggcaagctgtgtttgatgtggcgagtgggcaatcttcggaaaagccacttggtggaagctcatgttcgagcacagctcctcaaatccagaattacttctgaaggggagtatatccctctggatcaaatagacatcaatgttgggtttgacagtggaatcgatcgtatatttctggtgtccccaatcactatagtccatgaaatagatgaagacagtcctttatatgatttgagtaaacaggacattgacaacgcagactttgaaatcgtggtcatactggaaggcatggtggaagccactgccatgacgacacagtgccgtagctcttatctagcaaatgaaatcctgtggggccaccgctatgagcctgtgctctttgaagagaagcactactacaaagtggactattccaggttccacaaaacttacgaagtccccaacactcccctttgtagtgccagagacttagcagaaaagaaatatatcctctcaaatgcaaattcattttgctatgaaaatgaagttgccctcacaagcaaagaggaagacgacagtgaaaatggagttccagaaagcactagtacggacacgccccctgacatagaccttcacaaccaggcaagtgtacctctagagcccaggcccttacggcgagagtcggagatatgactgactgattccttctctggaatagttactttacaacacggtctgttggtcagaggcccaaaacagttatacagatgacggtactggtcaagatgggtcaagcaagcggccacacgggactgaggc

SEQ ID NO: 6 - DNA, 10510 bp, Homo sapiens
  source 1..10510: mol_type = other DNA
gcgcactggagccctggccagcgcgcagccttcccggcgccggcgggctgggtcttgggaattctggtttgctttggctcactcgctttttacaaaccactggatcttacatgcctctgtaccccccacttccactccatgtccccatgctcctgcgccagcaacaggtaagggctgctgtgttttcttccttttcgctcgctgccggtgtgtcctggggaatggactctggccgccgtcccctaccctaccctgccccgctgctccagaggtgcccagctgtcacactgctgtagcgctcacccacactccggagtcccagatcatgcctatatagtttgtgtgcgcaccagaacatccccagagctaaaaatactgagacatgcttggaacagttgagcaatagaaacagatttctctgcagcatctgtttgtgctcataacttccaatgcaccagctgtagcgagatactgggtttggaggcaagggcgagagcgtctgtgttcggttctcactctaagtggggaaaaaaagaaagaaaacgtggttgcgggtttgggggtgcgtcttgagccctggtcttaaaccttggcaaagcttgggactcagcactattcttgatccttggctttttttttttttcgtcattgcttcaagtatagctgtgagtttcaaacagagtgagtgtctcaaagttgccttcctgaaagaagagcgggagcccgagttggatttgagcctgaggatcgcaaaactttcacattacatatcactctcccctaaccaatttgttattttcagtaataatggtattaatacctggcttgaagacagtgtggttttaggaaagcaaaatagtcaaaacgacttgattctgcaaaaagattgcatcatagcgtggattcatcatgacaattggaaaaaaaatagggatagggaatgggcagtttaatgtctgtgtctgggtaaggatatcataggatgatcactaggataaaaggctgtgaattccgacgattttcataaattcagattttacctgttaggaaataaggtgttatttgaaaacctcggatatgcaaagaaaccatattgcgttttatcatttgttttcttttcacattaataatcaataggctgccagatttcctctcgccaaccaatgttgtgtgtctgctgtacattatctgtacacttgttagcgttttcctgctcacttccaaatgtttcatttctatttgccattatctggaaaaatcctggtgctcagaaggggtaagagatgttaattgaagcatggtggaaagggtgaggataaggtcaaggccattttacttagcttcctcctctctcatattttaatgacactttattttttaatgttcttggtctccataagaaatatttgatctaaactcatgtgaattttgcttttaaaaaaaataaaagaacacagtctttctacatcctttggattcctgagttgtagaatctctggttagctgtagcgtccttgtatgacttttgcgtagcctgaaagattgaaaacagctacctgcacagattaagtgtttcagatttttttaagtgtatatttttatttaccaaacatgtctgtttttcagcgtatgcaaatgctgagcagtgctacaagccaagtgtcgttgagtataaatgttagacagggggctcacacgtagatagatgggtatccccctcccctccccacttccccctcctcctccctcatgtttattgagcttctaagaggctgctttcctagcagttgtgctcacatagcaacatgtgcccagagcacactcaggctgttctatatggcgtcagctggggaatagctgctctaaacactcattagcagacctctctctcagcccagtacgttttcttttgctatctttatttcctatccttagaaactttcttatttattgcacaccatgaccatattacaggacacatttactctcatttgatttttttttttagttatttataagccatattggaagtattacctatatagtcaaagcaatttagatcccccatttgtaccatgtgatctccttgaccttacatttatccatagctctttctcctttaacatctacctaagtttaaaacgctgaactatttttttccattgcttatgcttaattttggctcagcctataaatagtcttaacatgaacagcaccacatgtaacctctcaatttcccaaaaatactttctacatcatgggttatggcctggataaaaatgattatttgtgtccatgaaatttacatctagacccttaaatattttggctggaagggagatttacacattgttcatcaaatcaaatttcaattctctggatttgtatcacttagtgttctcatctactcctcattgattgcatctcaaacattattttgaatatatgcttgaaataaagttgcatgtaagccctgtttttttaatgatagcttttctcagctgctcacgaattggtaattgctagtgagaggaccgggcaaggaagttttctgcttgcttaaagtcgctcaggcatgtgggcaggttaacagtaaaagcaatttttaccatgaaatattaagaccgttaccccatcaaaccttgacaggttctagactttagctatgtaatcagctccacaaaaagtctgcatacatatatgtgtgtgcacctgcatacacacataaatctgtttgggggcatatctatttaaagtggcaaaagttttctttttgccaaaaaaaaaaaaaaaaaaaaaaaaagcccaaaactcctctttttctctttccaggaagaaatggtaaaatttctaaatgatgctccacatgtggaacaaattctgttattagcaatggaaataagacaataatagtctctcaatacaggctactttcaagattctaaatgtggaaagctattcataggaagctggtattgtgtgatttgtggaattcaaacaaatgtcctggggtcaagatttttggaaagtcataattcagtctttagatttcatatggttgttatgttttaatctggaaggagtacagaaataagtcagaagaaatttgagactgtctactcatggcccctaatggtaactcacatggaaggattgttttgaatcatgaaaagggaatttcttggtgtcagcttataatacaaagttctgaagtcaatcagaactcaaaaatacaaagcattcattgaatgaagttcttctcctttttttgaaacgtatacctgcgtttggtgttgttgctgtctctgtctgtttagtaatgtgagaaaactaagatgaaataattgcttaaaaaatgctgtccatgtacaggatctagaacttcgaggggcaggattcttgtttctttggtccaagtgcaaacttcacagggaagcagacaaaacattgaagcaggtggaaggttaggaaagcacagtaacttctttttataaagtaattcaaaggcactttcaaactgagcatcatttaggtgtcaaaagctgccaaaatgtaacacacattgcaattttcaaagatgactaatctcaggaaaaatgtgttggggcacattcttgcataataacttggataaagaagaagtcaaaagacagtaacaaggcattttcaaaggcaatgcaccttgcctcttcttaagggaatgcacaaaaacaacataaatggattgctttatttgtttgcggctggcttggctgatatcggttctattaactgtttaatcttaatggatttagaggagaaggaaaaagtggagtcttcattatcaggcatcaacaaatttctgactcttttcatgagttattcattgcccccctttaatatagcagaaggatttggttgtatgaaaagttgaagatgggctttatttttcagttctaactcttattagttttgttattacacatttggtaaagaaaacaccttagactttctcttagctttttgttagttagtcaagggaaatagtttcttaggagtgaagatggaaagttgaagtacatagatagttcttagctagggaagactgaggcaataggagaaaactgagaactactatttgcacatattaagtaaaggtgagtgccttctccataggaacctataccaaataacagcgctggaagtgaaagtaagcggactggcatttatgatctgtccttacacagttgtctctgtctgagagaacacagcactggactcacatgaaatgagttcctgccttatctcaatccgctaaagacctggtcctttggcacaacggtgcttacttcctagcctgtcattacacatgacagccccatctcaggagaatttcagggctcttattgctaggcaacagaattgctctctatcccccagagcaaacctgcatcttcagagtcttcaagccagaattacctgtactaatagagacttagcaggcatttctgcatagtggttgcttaaagtgtgagaagtgatgaagcaagcccaaacagtatccagaaactgggatccatttcagattattataagcaataggggcagtattcaccatcgtaggaggttttgtccactagttccctctattttcaggatacacactcagtcaagcctactccaaaatccacttcactattctatttgtaggtgggaatctgactttcttcctctagcatttcgtcagcctatccggtctgctcagttatatcatttaaacaaaatagtctgtcttccgatttcaaccttgccatcagggactctaatattctcccgtaatgcacatgcaactcagtggatatcaatgctgcgtccacgtgaagttttctaaacatgtcctttttccaaaaattttacgacaaaccttggaatcagacagtctttcatctgcttaacagggatcctagcaagtggggcaggcagaagaagagaataagagaagggagaggtagacctgaacaatagaatattctagatccctggccaaaaagaagggaaaggggacctgacctgacattttggttgaacaactgtcacagacaactcttctatgtttaacgactttatgcttttaaataaaagttttacaaagcatgcttaggatacaaagaaatttaaaaccttataatttaatgtaaattatacttatctttacctgattatgtgtttttgttaggatatattgtcttagacagtagaattctttaagagtggcttattttgtaatgcacagtctctcttttcttgcaggacatgttctctggatgtcagctgagtcattaaagtaactctgcatgtcagtagacagaccttggtagaaccacaaggctcccagagacacccatctctcctcatttttttggtgtgtgtgtcttcaccgaacattcaaaactgtttctccaaagcgttttgcaaaaactcagactgttttccaaagcagaagcactggagtccccagcagaagcgatgggcagtgtgcgaaccaaccgctacagcatcgtctcttcagaagaagacggtatgaagttggccaccatggcagttgcaaatggctttgggaacgggaagagtaaagtccacacccgacaacagtgcaggagccgctttgtgaagaaagatggccactgtaatgttcagttcatcaatgtgggtgagaaggggcaacggtacctcgcagacatcttcaccacgtgtgtggacattcgctggcggtggatgctggttatcttctgcctggctttcgtcctgtcatggctgttttttggctgtgtgttttggttgatagctctgctccatggggacctggatgcatccaaagagggcaaagcttgtgtgtccgaggtcaacagcttcacggctgccttcctcttctccattgagacccagacaaccataggctatggtttcagatgtgtcacggatgaatgcccaattgctgttttcatggtggtgttccagtcaatcgtgggctgcatcatcgatgctttcatcattggcgcagtcatggccaagatggcaaagccaaagaagagaaacgagactcttgtcttcagtcacaatgccgtgattgccatgagagacggcaagctgtgtttgatgtggcgagtgggcaatcttcggaaaagccacttggtggaagctcatgttcgagcacagctcctcaaatccagaattacttctgaaggggagtatatccctctggatcaaatagacatcaatgttgggtttgacagtggaatcgatcgtatatttctggtgtccccaatcactatagtccatgaaatagatgaagacagtcctttatatgatttgagtaaacaggacattgacaacgcagactttgaaatcgtggtcatactggaaggcatggtggaagccactgccatgacgacacagtgccgtagctcttatctagcaaatgaaatcctgtggggccaccgctatgagcctgtgctctttgaagagaagcactactacaaagtggactattccaggttccacaaaacttacgaagtccccaacactcccctttgtagtgccagagacttagcagaaaagaaatatatcctctcaaatgcaaattcattttgctatgaaaatgaagttgccctcacaagcaaagaggaagacgacagtgaaaatggagttccagaaagcactagtacggacacgccccctgacatagaccttcacaaccaggcaagtgtacctctagagcccaggcccttacggcgagagtcggagatatgactgactgattccttctctggaatagttactttacaacacggtctgttggtcagaggcccaaaacagttatacagatgacggtactggtcaagatgggtcaagcaagcggccacaagggactgaggcaagcacaatggtttcaaagaaagactgtaagctccatgattagcataaagcactaaccatgtctccatgtgacccgatggcacatagatgttgtagaataagttatgggtttttatgttttgttttgtgtttttccaaaacttgaacttgcaggcaagccttggttgggtatttgatttatccagaatgcttctctttagggaacaaggatgtttttaatggcataacaaaggcaagactctgccttaatttttgaaaagctgctaactacatgaacacaaatgtgtatttttgttgcagtgtagttttccttttgtgtaattttaaagtcagtgttgaattttattgaaagctcatgatgcgcttcaaagtggcaagtatttggctattaactgccaaaacaagagcctgattttttgaggccagtaattcgtttgctagaattgattttttttctctctctctttgttacataagggcattatgtaacactagccgaatggtagcctctgggttgttgtttttttcttttcctccatgatgttaatgggttatctcaaattttaagttaaactacctaaaataaataccaaagataatgcatatttttgcacagtggagcttacacttaaaagaaaacaaagccccatgggctgccttgaaatcaagagacaataactttgaacctcagcaagaccttgaaccgccggttcattttgcaccttattcagaaaatagagcatcatactcaccgagtctagtcagtgtagtgcttttaaaaattttgtcctttcatgtaacttttttattttaagaggaagaagaagaaaggggcacacacacacaataccgacgtctatcctttcctgctaggcagtgctggccaggctcatgtgtagtgtgcgagatggtgatgtactcttatatttttctgggcttttccttttgcacattccaaaattcatttcataagacaagatcttcataggacctccttggcatcctggcattctcaaaactgagccatccagcatgaaagataaatgggtttaaacccttgctgctgaatttattgcctggactgtcaggacatcaccagcccaccttcaccttagggaagatgccacacctggcctccacacttgctcttctgatcagtctgtctggattgagtcctacagtgtcagatagggcggcaaatgccaaagcagggaaacagggaggtgtggacaagccagtttgatgcagcacttcagatcaagtgcttaggaaggagaggaaacttgccttttttatggcagaggatagtaatgaaaatgtctcagtattttagggtcaatgagagccataaaaatataacataatcacaagtaaaggagataatggtctaaaacagctatttcccttttctgtgtgcatacttatgactgaatgtgagctaagcattttctcctgtggagccctagagcaggttactaaggaaggacacattgttttccagaagcctcccctgcctggctgactgccttgctagaaacataattttttttttctcactgaagctcaataatggaactctttttttttttttttttaatttaaagttccctatttgtgaattctgggattactgacttttctttttaattggagtctcaaaatcaactctcttatggtattatatctctgtatgccattaaaaaacagcttgttctagaatcatgtattttgtaaactgatgtttgtgatggtctctggttcttgaacagccatatctgaatgccgtgcctgcaaaactatgacaatttttgctgttttcagccttcagatttgatggcttgggaaactgaggtgttattttcaatgaaacaaagaaagagatgttaagcaagtggttgttttagatccaaatgtaaaggcaggtttgggaaggtgtttaaagagttggaggaattggggattgagttgtaaagaaaacttacagaagaggcaacaatttggttcttgacagtgagaggatattgagggcttcagctgctgctattatgatgttttgcaaaggaaaataatcaaaccaaagagtattcagtgatatgtaaattaaatgaagatacagtggagaatgggggtgaccacaaaagaggctccccctaaacacacagtgctgccacttaaaaagacttgagaaatttgaaagggggtgggtatggggggggcaagaaagagggagggaaatctttcaacttatttctgaaaaagagaaaaaaatataaaatttctggtgcacaggtttgttttttcaagaaaattttgcagaagctatgtttttaaagtgtacattttataaagtttatcagatattttcatatttaaagccaaatgtaaatagaggtctgtaaagaaaaataattgccatagaaagtataatttcagtgcagtaatttctgagagctagtacctatatgctaccggttagcatggttttagcaaatatataccagccttataaggttcgtattgctatgttcttctgttatttatttcagcatggactgttcatttgaaacctttttctagttattagcgttttaacagttacaagctttaaatggcaattttttttttttttttttttttttttttttttttgtcaagagccaagacacaggtaatgcacgacattgattgctgcattttaccttcaaaatatttgtccttattgactgggtctccttaattaatgtacacatgtcattagaatgcagacggaggggactcaccatgaatatctggggttgattcccagatgtgtgttgcttctctattgcaagcagattccctgttggatttacttcggatttattcccttttaaagaatttttgcccatatctggaagggcactatatttttgggaggagccatagattcctggttatcctatttttaaacaaaatgtagacaaagtgaactctattttgattattgagaaaggagtagttttctatccctctaagagtatacttgaatcagacattttaaggatgtcactatggcactgttgtcatttccaaattcctagaaaagtttgttttactttgtttttattctgttaatgcattctttcttctctttacttcctttcttaccagtacactcctatctcaactctgtttatttgatgagttctgtcccgtaaatcatatttcccttacaattaataaatgtcacttcatattttataataaaccactcagtaaaagcaaaagcttgtcctgagaagtagagtgagttctttttcactctgtgtctaataatgttaaggtgggaaaaaaaaaagtgtggcatagctacctgcccatccccaaccctcagcaaagtagaatctcttttctggtaattttgggtttccgctctgggctctggcaagttgaacaatcctagccattgacaatcgtgatagttattattttcccatttgctgtctttttgtatctaaagtcttcctattgtactgcacaaaccatggattgtacatatttttatatattatgtcttattttattatttctaaataaaaaaattaaaaattgaaaa

SEQ ID NO: 7 - protein, 1159 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 8 - RNA, 4070 nt, Homo sapiens
  source 1..4070: mol_type = mRNA
acgcggcctgctcaggcctccagcggccggtcggaggggaggcgggaggcgagcgaggacccgcgcccgcagtccagtctgtgcgcgcccgtgctcgcttggcgcggtgcgggaccagcgccggccacccgaagcctagtgcgtcgccgggtgggtgggcccgcccggcgccatgggctcaggatgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttagtggggctgcccagtgtggacacgtggctcacccagggatcaaggcgctgctgggccgctccccttggaggccctgctcaggaggccctgaccgtggaaggggagaggaactcgaaagcacagctcctcccccagcccttgggaccatcttctcctgcagtcccctgggccccagtgagaggggcaggggcagggccggcagtaggtggggcctgtggtccccccactgccctgagggcattagctggtctaactgcccggaggcacccggccctgggccttaggcacctcaaggacttttctgctatttactgctcttattgttaaggataataattaaggatcatatgaataattaatgaagatgctgatgactatgaataataaataattatcctgaggagaaaa

SEQ ID NO: 9 - DNA, 33359 bp, Homo sapiens
  source 1..33359: mol_type = other DNA
ccgcggccgggccgagccgcaggcgctgcccgagccgcgggcgctggagcggctgtcggcgcggtggcaggcgggagccaggagtccggggctcgggcgggcggagcgcagcgcggggacgcggcggagcgggccccgcggctcggcgggggccggcagacaggtgtgccggcggcgggcggctcgcgctcaggcggctccaggccgggcccggggtccggagcggggagccgggagccgagcgaggacccggcgcccgcagtccagtcttggccgcgcccgtgctcggcttggccgcggggtgcggggaccacggcccggccgggccacccgaagcctagtgctgggccgggccgggccggggtgggtgggggcccgcccggcccgcccatgggctcaggatgccggtgcggaggggccacgtcgcgccgcagaacaccttcctggacaccatcatccgcaagtttgagggccagagtgagtgggggagggggctgagcggggagggggctctgggggcgggccgagctcttccgagtgtggatgggggggcgtgcccgggccgtgtgcgtcggcaagtgcgagtcaatggggcctggtgggccccaggctttggcgaggggacggcgggggcaaggcttggtgcaccggggccgtgggcaccgggccaaagggtttggggttggggcttggggcgggaaactcagctcaggcttttgggatcggagtgtgtgtgacacgctgagtgtgcagggaagggatgtgggatggagtgtgcaggtgtccccaactctgggtgcgtggacgtggatggggctgtgtgcaagggcataagagtgtgtacgacccaaaccctgggatcccgagagtccagcgcgcacactggtcttgccagatcttgactgtgttgtgccggagggactgaaaagccgtggatgcagggtcagctcataatctcttcttgtgtgtgccgtgtgtatataaccctgagcccatgtgtcacccgtgagttagaccctgtatgggcggtggggaggtgtctttgacaaggctgcgagcttgtgggctctgtggagacagggtgtcctggggctagagctgggctttgagggtaagcagctccctgtggtacccccaagttgtgggttttcttgccctggagactgtcacctaattcccttatggggaccgggagtacagcacccctaatgcctgcctggtagggaggcttctagaagggtcccagattaggcaaatgtcctgggggtgggcagcattcatgtgcctgtccctaaggctgcatgagccacagtgggtgtgactgccaagggccctgggagttgggagaggatgacattccttgtccatgaccctgtgagcacaggacaacgcgtgcttgtaacggtggttgggggaaggaggggtcagtccgatggtcagtattgtgtgaagcggaggaacaagatgatcctgagggggtggagacaatgacactggttgagggcctccctgagaggtgggggtgggcccagggggaggctgtgggcgggcctacgaggaggctgggatggatgtgggggggggtgctggattctagccctacctttgccactgactgccatgtgaccttgagtgactcttccctctttgggtctcaggttccaggtctgtggaggaggggcaggctttctgtcccatccctgtattccaggatctcagtgaagggtgtgtggtggggtggggcatgccttcgaagaaacttgctcttctttccccagcctgggagagaggacaaggggtggtttttcttctatgaagctggggtgtgtggctgggcctctggactgccttggactgtggcagagctggaggccgagttagggggtcttttcaggtgacaggaaaccctgactcctggaattgatggctgcctgggaggtctggggggccttgggaactggccaaaagatgaaccagttccaaaaggcaactttctgggggccaggggaagggagaatcgagaaaggtgggaggaggccaagtcctgatggcctgggcctatctcagggtgccctgctgcagcccagctttgcacgtaggattgcctagggctgcaacctgagggctatgcttgctggggaggggaggggaagggagggggagaattccaactgctactgaaagccccagcattacaggccaaagtggccggaaacttctgtttataaaaagctcttatcccaggagcatcttgcctggcacaagtgaggctgctgcctgcctgctgctcactcgccagcctggctctccgggcaggcctggagtcagggagccaagtctggcagaaatggaaccatgggggtgctcatccaggatgaggccaaactgaggaaggcctcacatccctgggcaggtcccctttcttgggtctgttttcctacaagtggggccaaagtctcaggccccgcccaggctccaggtgtgtccagggtttggagctgcaggtccctctctttgcttcctatgaccttgggcttctgcctgtgaccctcagcttattcttctgcctctgccagaccaaggttcaaaagctggctctatcatttacaagctgcaagacttgggcatggtgccgaacttctctggagccttggtttcttcatctataaaaggaggataataacacctaccttatggggttgtcagaggacctaatgagatgcagcgtccttaggtgttcaacgtgtaacttccctcatcctctctggcacacccacccctcagctcctgactgcccttggggcgggggtggtgtgcagaagttctagggggtactgacctggtaggatcctcagataatcggggagtgatttcctagggagcctcaggataaggagtggctcagggagcgccccccagtctggagagcagggaaattaaaagcagaatccagagagccgatgcaaagggaatcctggccgccatctctggcctttcccaggctccctttctaggcccagtgtttatccctccgccgcggctgctgacacgcgttgctgacacccgccaggctggcctcagcctcgggcatggaggaggcggcgggctgctggcggcctgcccgggggaagccacgcagcccgcccgcgggccaggcggaggagggggcgcgccgcgcggcccggccggccaggcacctgtatcggggcacacgcacagctgcgttcgccccctgctgggaccccagtcccagccatgcttcctgtggcggggagtccccgggcggctgtgtgagtggagaatgtggggaagggctgggagtcccggaggctggggtccccgctcacgcgcactctcctccccgcccccctaggccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatggtaggagcgggccggggcggggccacgaccaggggcggggtcaagaggggcgggaccacggcgaggggcagggtgcgtgggggtgtgggggtgtgggggcggctggagaacgccccgagcccgggcagggttctgtgggggtgtgacgcgggcgggggccctgcggtgtgcctgtgggaggcgcggttccggggcctgcgcctcactctgctgagatggttttcaggagggcaaaggagttgctaggctgtgggtctggctcctctggtcctttttttccctgggcaggtgtgggcacgtagagcaagttggactagagttagaccgagctgggatggcaggtacgtgcgtggatggaggttaaaggtcagattggcagttgtgcgctgacatccgggcacaggcccctatctaccttagggcagttgctgccaggtgacaggtctgggagatgtcatggagggacaggagggctgcaggactctggctgtctctgctgcccccctcctgccctgcgcttcagcagcacctgggggaggtgcgtgcctctccccgcaacctcagttccctttgtctcagctgtcgagcgccccgccctgccaccctctcctgcgccaggtgtctttactcagccaccgcctcccagcacagctgctctgggcttccggagagcggataaacagagcccggggtggggctgctcccacaccgtgaatgccctgttggggtgggctctggtcagggcgggcagagcaggaggactggggtcctgtgggcagaatccggctgcagaggcggctctggaacgcgtgcaaaccttgctgcttcctagcgtgtgtcccataagcattgactcaacccttgtgagcctggcaggcactaggctcttggagatgggcaggacagatgaggctcctgccctcagggagcttgtctgctgtagacagtgagcacgctctgctttctgtttgggaccaggccagccagccccatgtgccagccctgtgtgctcagggttggctggggtggccctgcaaagctttgcatcctccaggctgtgcttttgcaggcgtgcacagtctgtggcatcactggacggcactagagggggcatggtccctgcaccctgtggaagaaggttcgctcacagcaaatcagggttctaactggcaggaggcaggggctgtgagactagactcaaaccctgaaggcagtggatggtttctaggggctagacactgggccaaatgctttacttctcttatctcaggcactcttcacaacccagggaggtagacactactgttgtcttcattttgggggatgaagaaactgaagttcacacagtcacacagctagaaaatagcagttgggaacggagcccacatctgtctgaccggagccctgtgcgctctgttctgtctgtcatatattgaaaatgacaccaggtagttgcaggtctatctgtatgctgtacattatctcatgtgaaattttattgtaaaagtttgccaaccacaggtgtactgaaaagaatatgaacccataagtcctgggtttgtgtctaagacaatgctggaaattatacactactggacaagttggttgacttccctgggcctgtttctacttctgggataacgtgccttgctttggaccttcacaagggtgctgagctttcatagagagtgcctgcaacgggccgtgctctgtaaacacggtgccagtcccactacgatgtagctgtgctggggatttggcctcttggcagcagcggctgagggctctttcctttggctgtggtatgtgtggagtgctcttaggtggagctggccctccctcccagtctaggctggccccttctggagaggatggctatgggcccccttctgcagaggagttattgtgggatcacctaatccaacccattcgacttgcaggtggaaggacggagaagcagaggtctgatgaccagtgaatcagtggaaggagtgggattagaacccagatccccagcccaagttcaaacttgtcccctctccaggttacaactccaaaaaatggggctgtgacccagaaaactctgtgccagcaatctctgtctgtccccttcttcactctcacctagtttccacggggctcaggtgtctcctgttggccaacaggctggcctggcactcagccacctaggaccccatgtatcctggcattctaggcctggggtgatgcagacagatttcctcagcctccgcttcttctgccttgaaacactcagtccctcctgtgatccccgggacctggcttgggctgcgtgtgtgcgtgcctgtgtgtgtactcacacatgggcctggtagggaggcggaggtgaggaatgcctgcgccaccccttgtttctgtccaggcctttctcagggtctctgcctgggccagcctcgggtagggggagctgggcaggaggtggagccttctgctggcaggcgccgccgtggacagctgtgctgtgttggtgcctctagccttgctctgcgcagtgtcagggcctggagccagacagccccattatttagttgctgggactgctgggaggtggggaggctagactgggggagtgtgggctgaagggagagacatgaggtgcctggggctcactggccctgtgccccttgaagtcaggcccgggattcaggtggaattgggggcagagtccagtggtgtgaggctggctggcggtaacatccccgcaaggccctggggaggcaccccgggcagcaggtgctggctgggtccacacctgtgctcccacatcgccatgggggctgtggctcctcctctgccagtcctcacagagtcccttctctctgtccccctgcatcctctctgctctttgttcctgcctctgctccctgcttccctgccaccgtgctcagccagcctgactgtgcccaccttctcggctcctctctcactccccacatctccttgcctttgtcttctgcctgcctggcttactctctgtcccttggtctttatctcctctctggtgcctcctcctgtcttcttccctcgcctcagcaccccggaaccttgctccttgcctctctccctctctcccattgtcccccacccctcggagcctctatctggaaggctctgtctggctgctcatacctctgactggcctggcctggctccatgtccctgtgtccccgggagcttgtgcgtgggtggggacggtggtgtctctggagaggagtctattagcttctccactccagctgcaggccagttgccatggtaacagggtcagagggccccccactgcaacagctgatgctgcggctgttgggagggggtggggacctgctgtccaggcctgctccccagtgcactgtgcccagggcctcagaggtggccccacgcccagtgggcttcggtctgtccccggccttggaacccagcctccccagtgctcctgcaggcctcagacctcagactgccttgaaatccttagtacctcctgtgatcagccccactcctctgggcctcctgcccttctccgtcccccaaggccgtctgtcctgtcttcttccttgccttcttcatttcttcctgtgcccattcctttaaatcagacgccctgctcttgtcaccaccacgcctcaccttctccctcatggctgacccctccccagctcagacacccactttctgcttcttttcagccccctgggtctgagctgaggatgaaagaagcttttccttgaaggggagccttgaggctgggaaggcaggtgggacagggattggtgtataagtgggttttcttgtcttgagataggaggcagaccagagcgatcccagaggatggggcttagacacaggagaacttttcctgtggcactgaagctatcaggggagcgcccttcatcatggggacagcggtgttcctatccttctggtcaaggcctggtgcagcctggagacagggctgagtgggagaatctgcagggctgccaggtgcgggcgggggtccatgggggccctgcttccgagccagttgcctcttgccctgcagcaccttccctccacctttcccttctctagctggatcagaggcctgttggaggggctccacgggggtcattcctgagctgcggctgtctgtcctggtctgccggggctgcagtcttcctatcccagtcctgctccaggggtgaggcaggcatgacgcaactgaagggtgctatctactccttgacccaccctggggtctggagagcgtcgggagagcggcttctccgtgttgcgtcttctgcagcccccggagaggagctatgtaaatatttgcgaagagctgtctgagatgggagcagagtttgatggtggccttgggctctggggaggggggccatggggcagcagaagggccagcttgggggtgtgtgaggggggagactgggcctcttttctccccagaggtggcttcctgggtggggtgattttggaggttccaagaaagcccttcacatgttggtatttccctgaagcgttcctgaaactcctcccttagagaacttctgcgtttagagtgctcctttttccttaaaaacacgttctctctcattgccccctcccagtatagagacagaaagtatttagccctttagtgtgagggagtttaacagaacttccctcccgacgtgggcaggagggccatgggagtcagggtcagtagaagagggaggggtgctgggacaggtgatgaatgtggggtgctcccctaaagtcccctccatgtccttagtcttgcccataaagatctctatgctgtggcctgtggttggcctcagataaacccctctcctttttgggtgcttatccaggatgagaaacaccttgggagccaggcagccttggtcctccgcctcctgcagtactggaggaggttccctgtccctctccccgccaccaggccttgagctccaggtgggtggagagcagcccatctgctttgttctctgttgtatctgcgtgctcagtgcacagcgagagctcggcactgtttgctgaatgaatgaatgaatggggtgggcagtgtgctgcgttgcaggagccttccctgggagtcaggtgacttggcaggagtttccagcttggttacagacttgctgtgtctgggagtcctctagccttgctttgcgtggacattgtggggcacaggagcatctgggggctcccagcttcaccctcagaggtggaccgaggaggatggggtggggggacagagtcttagagctgccacagagagcacagctgaggtgggggtgatgccacctgaccagcagcacaaggcacttctattgttgtgactgcactttgtagtttgcaaactggggaaccatgctttaccgcaggtggtcctcatggccctgtgaaaggcaggattcctctccgtcactttatgggagggaagctcttgcttagaaaggctgaggcacttgcccaagtttacccagctggcgggtggcctgagtgggaacccgagcctagctgtcccctccctgcccagcgtggaatgccactgcaccagctctcttgcccccagagagcccttctggggcatgatgggctcagggcctcaggaaacttggattttagctggggtctgcaaacctccattcctcgtttgctaagtgggatcatagcgcctgtccctgatgcactttggggtgtgagggtgttcctggaatgagatgacagacacccacaaagcagcgtgcggtgccagagctcattctcctcgccatgaagcagggcgtcacctaagtggctgcagctcctagcccgtgctgttgcaatcgtgggaaatgagcctgatccggggcctcagcacccctgctcctatggtgcaccccgccccatcctgcatgctgcctgcctctccccttccctccctgcaggctgctgctggccccgggtcctgcctctcccccaaataattgaaaccacagcatttgtgttattgctttttcctttctctttcatttttttccttttcccttttatctcactctccagtctcattaccctccccataggcaaccgttcaaatgtacttactgtgaatgtgttgcttgcatatgttcttgcaaatgtgtattttgtctggatatatgcatatagatttaatttatgtaaatgatattgtgctatatgttgtattttggtgttcgcttttcttacttgggaatgtgctttaagtccagctgggttgctgttcgacatctagtctgtaggtgcgtattcagtggcatgcggccgccacgcttcacccacccactctcccggggagggaccccacaatgtctccaactcccagccactgtaaatgaggctgtagtgaatatccttggacatggtcccttctggacttgggtgaggatgtctgtgggatacacgcccgggagtggaattgctggatcgcgggacatgtggacacttaactaagtactgccatgcctgtcatcaccctccttctcagagctcgagattcccatacccgcatatcttggccactctcgacattatctacctttctaaattttgccaatctaataggtgacagtgatttcttgttgctttaatttgcattcctttgatttctgttgagtctgagcatctcttaggcctggtagcctcttgggcttactttcccgtgaattgcctgctcatgtcctttgctcattttaattttgacgtcgccgtcttttgtccttgttgatttgcaggagttgcttgtgtactctagagataccaatgcatactgttaaacattcagtgtattccagattttaattccttgttgaactggaaacagatattgcaaatatcttctcccattcagtctctcttcaatttatccaacaaagatacttaattttgatgcaaacaaattcattgatttttagctttgtttgtacttttgaagtgtctcaggctgcagagggtctccattttcttctgttctcttaattgttttgctgttctcagttaggtctataatctatcctgagactaccccgtatgtggtgtcaagtagggatcgagttttattttttccatgtaatgtgccaactttcccaacgcatcttgtaaacagtctgttcttttccgctgaattgtggggcagcctttatcaggttaatctcccacatagccatgagtctgtccctgtgctctctgttctgttccaaatatttatttactggctcttgcattgatatagcttaacaaaaacctatagccttccgtatgtcttagcgtctgctgaggcaaatcctctctcttcattctgttttttaaaggctgacatagctatttgtggatctcccttgttcttcataaactttatttattaattaattaattttttcagatggagtcttactctgttgcccaggctggagtgcagtggcatgatctcggctcactgcagcctctgcctcttgggttcaagcgattctcctgtctcagcctcccgagtagctgggattacaggtgtgcaccaccacatccggctaatttttgtatttttagtagagacggggtttctccatgttggccaggatggtctcgaactcctgacctcaggtgatccgcccgccttggcctcccaaagtgctggattacagacatgagccaccgcgcctggcctccctttttcttcataaactttagagaaagtttattgaattcttggaaagaaaacacccactagattcttgattggaattagtttggttctatagattaatttggggagaactgacatctttatatttgtcccactcaagagcctggattgtctctccatttaccagatgagttctgtatttcttgctagtttaaggttttgttcccttttgccccgactccaacagggttcttgtgccttcttggtcaattcctggacacttaagagtttttgttgctattgtaaaagacgtattgtttttgactatgtttcctaattgcttgtgtagaaaaatgctttatatttttgcgggctgatcttgtatcttgcaaccttgctgaactctctgatctaattagttctaattgtttatctgttgatttcttgggggtctctaggcaagtgaccccgtcatctgcaaataatgccagttttcgctgtttatgtttggatcaccgtcacatgttttgatgctcgaatgttccacctgactttctgggggcttttcattagcactcgtatttgtttgttggtgcttcacagtgtcctgtgtgtgtgtgtgtgtgggggggggggagggggcaaatcaatgaggaattataactcttggtttaagctgtgactcatggcagagctggaattgggacagggctttatcaaagcttaccaggctgtctggggtatgctaggagccaagacagagcacagacctagaagcctacaggccactcactagaggagaccccagagtgtcccctggtggacagtggcagctgtgtgcaccttcactgccctgtggcaagctgtgtgtggggtcacagtgaaggggggctgaaggggactgaggctcagaagtctcacacctggtgcagttcctgcttttgccgccttcccttgggttcctgggtgctcagcctctgtgttccatgaaaactgatgaccaggttgaattttgcttcatcaaaaggcaccttaattcccatggttgctccatctgctgtccagacttgggctggagggcaccacgggcttgaaatgggcaaattctgctgtgaaagggtgggctgtgcccctggatctggaagggagctgagagggtggggacatggcctgtccccacctgttatgactgcagatcaccacccctgagtccaccctcagttcttctgcaactgccagtcctgccctggcctcagggctctgactcggatggacggggaagcccggccagtgtacagtggccttctagacgatgggcattgtttggacaagttctccaagagcagcgtccatgtgggtcagacggagcagtcaaggaaacctttctagagggggtcctagaaggctggggaggagttggagggaagaggggtgggggccatggggagcaggagggaagggctgaagctggggcgcaactgcccgggactcacgcgcctgtgcagggagctgtggagagctgcttcctggcatcagcgtgcccttgtttcaccttcccaggcttctcagcaggagggcagcgggtgggacttccagttcgtgcaccacccgagaggcagagccgggccccagagctttagagaagctagaggacagagatcttctgaggcattgctgtggtccggggcagagatggctcaggcatgccacatgcagtggatttgggtgggtctagatgactacagcggcccccaagaccctataaatgatcccatggattgctggctgggtctcaggctggaaggggtggtggaggagtctgtctggagatgctgcttctgtggggtcagggtctgagcttggggcccctcaacctgctgcatttcccagatcctggaggcaggagcttaggactggtctctctctcattcagcacacacacacacacgcgcgcacacacacactctctccctgtctctgtctctctccctccttctctctcattcactggttggcctcttgggctccctaactctgtcccctgggcctgcaatgtccctggcctttcctaggcccctggctgcccatgagagctgtgttagtgacacggtggccccctgccaggagcggctcctgctgcatcttcctgtcgccgccaccaggctaaatataacctgacactgcacagccctgggctgctgctccggagacagcctctctgcctgggggcggacaggccgactggagccccagcctcctgccctcagcgtgactttgggcctgtcctgggcacagctcctcctgctccgagcgaggcagggaggcgactagggcgaggatggctgcctccctgaggtcaccttccctgcagggacccagcaccccgccttgttagaattctggatgcttccacaaagccatctgtcatctcatctggcacccagtgcaggaatcacttgtccccacagtacccgcagggagcctgaagcccagggaggtgggtgacctgcccatagccacagaaaatagagggaacgcaggagccaccatggctggggtagagtggctcggctccctggggctcctccacctgtctggcttgttccctctccttccctcttgcccatgcatcaaactggggtcctgttggatgggtggttcactgggggcaggtgccaggcgtgtcttgttggtcacggtggctcaacacttcgcaggcacggatgtgcttagtgatttgcagaggactgggaggagctgcctgggaagaagctgtgatagcacctcatttacggaggagcgcacggagactctgaccccagaaggtgcctaaggtcactcagggccatctgcagcctcttcctccccacctggtgctgacatgggagataaggcctgcccctgccacagccaaatgtggctcctgcctggggcctggcctaggctagcggtgcccagaggtggggaattgggtggagcccctgacctcggatgggttgggatgctgggcaggaacagtgccccccaccccaggtagactgctgggtgtggattggcaccaaatgaaagcaagccggcgttgtgttctgagaagtgatggacaccttctgtggtagcaggatggcgctgtacctgccactctgggaatgtgtccaggcctggctctcccgctctgctctgagctgcatggcctctggaggcctgctaggtggccagcctgcctgcctcacccaggccattgggcccaaaggcacgggggttctaaggagccatggagcctgtgcagggatggttgcgtccatgggtgcctcccctttcagagggcccaggaagcagaactgggccaggcagggagaaccttctcacccaaagtaaggtgtcttcagccagcgctggggctgaggctggggtgggagtggggtcggggctggggctggggtgggaatggggccagggcagagtgttctggcattagccacatgagccaggctggccctggggcctctcattgctaagctccctccacccttgtctgcctctcttctgtctcaagcttatggtgacctcaagccaggctctgagcttcacatatcttaggccatttcacccctacggccctagggggtgggcaggatcaccctgtgttgcagatgagcgccccggggctctgggccggtaagtgaccggcccaaagtcattctaatgcaatgtgcagaggccacctgactccacagcccatgccccctccctttgagactgggaaccctctcccctgttgcaccccctccgccctccttgtctgtcctggctgccccctgagtgcgcccagtgctggctgcctcgggagtgtgggctgtgcctgccacggagtggctctcagcggaactgcagcctgcagcagctgggtccttcgtctctgccctggggagctggggccactggcaggagccagggcgagtcggtgggagaagggagcagagcgagggcctgggggtctggagtaggtggcactggggcaagtgaagggactcccatccccccataacccagccagtggtgggatggagtctggctgggaggcctggctgaagagctgggagcaggtgatgtggcagttcaccgaagccccagccgacgcggcagggtggttgcaggcagtgcctctgagagcccaggcctatctggggcccctactttccttccttccacatccccttgatgatcgctagatctcccccgccccagcccctgccatcaggcagggctccagttcacattttttgaataaccaggcctttgacagggttattttgaaaggaaaaatttcttgaatctcgacaaaggaagagggccagcagtcactcctctctctctctctctctctctctctgtctctcgtgtgtgtgtgtaagtgtgagttacaggcttgtgctgtgaggtggcgtatacctagagtttctgcgaccctggcaggggattttgagatgtggggtcctggactgggtgggcttcagaatgccattctgggctgtcttgctgagcagcttgagctgaagaggggacgggctgctgaggtctcaaggttggaatttctggggccctgtgttggggacagtctccagcccattctgaagtttgagtccatcttggctccagactctagacttctctgaggcaagctccctggtggaggacttaggttcgagggcctggctggctgtggtccctggggtgggtgctggggtctcttgggctaaggcttcctggtctccagcctctcctcagtcctggtgaggcgcatccctgttcaggccgctgccggccatcaccatattagggcataaactccaggcaggctgtctgggtgccaaatggtctccagctgggcccagccccctgcagcttccaacaccactgctgtgtgtcagaggccagaggtcaggggacaagagtggctgctgcagggagacccagacagagccccagagctcagggccctcgggcggggctgtgagggtctgggatgtggtgagtggaagagctgtggggtggggcggagagttcctggccagccccttcacctagtgccatcctgtttaagctggtttcagaagacctcgttcccctagatgaatccccagcgggttagaatccccgagcctggccagcatctgccatgcatcaggcccttcccacctggcctgctccctgcctccccagcacgcagccccttgacctcactgtcaggcttcaagcaccagctccaagtcccaggggtggcgtcgcctccaggttgccggtgtctgttgccggcttctaccttgactcacagcaccaccactgtcgcccctgctgtgggtttgagatcaggagtaggggtgagccagggaggctctaggaggtagaaaggtgaggagaggctgggtgcggtggctcacacctgtaatccttgcactttgggaggccgaggcaggcagatcacttgaggccaggagtttgagaccagcctggccaacacagtgaaaaccctgtctctatcaaaatatacaaaagttagctgggtgtggtagcatgcagctgtagtcccagctacttgggaggctgaggcaggagaatcacttggacctgggaggcagaggttgcagtgagccaagatcatgccgctgcactccaccctgggtgacagagtgaaaccctgtctcagaaaaaaaaaaaaaaaaaaagtgaggctgggttgctaagatggagtgggactgcatggtttgtcacccttgcctctcccccatgcctgcagcctccctgtcagcatggcctgtcactgacaccaccagccagtgctggccccagggcgtatgactctaggtgctaggcactaacgtggggacgggagacactgggaggcagagaagacagctgagcagagggggagctgagaggaaggaagaggctccctagggagagtgggagatgaggagcctgcctggtgggtccctggggggccatagaggagctggagcgggctgttaactgccttctggtttcattaaggacttcctgtgggcaggaggttctgagctgggccttaggtggaactgagtgcttggcctaaaaggggctccttcagaaatcagcacgggttggggggcggggggcggtggggcagcagggggcccatgtgctcaacagggagctgagggagaccaggggtgcttggcttaggggactgacctcatctggtacagggcctttggtggacagagagagactggaaaagaaaaagtcaaggatcgaggggaacgcgaggcgaggtgaagatgaaagatctagaagcagtgcttcttagactctttggtgaaggatcagtgttttttaatgctaatatgttataggctggtacatctgtgaaatacaagagcacatgcgtggattttgtggtgatgccaaattgctgtaaagtttttgaaattcttgctctcaacgtctgtccttatcttgtgtgctgacacaacggctggtgctaatctagagagaacagagagacagatagtaacagctaaccgtctgtagtgtgcggcatacgccaggcattgcctaagcgctcagcatgtgctgacttggttaatctttataactcatgagaaaagaattatatacattatgtgtatcacaacatcactgtgtaccccataaatatgtacaattattgtgtcaattaaaaagttaaaaaattttaaaaagaaaaaattattattcttcccattttatagatggaaaaaatgaggcacaaggatgttaaataatttaggaaaaaagcataagtgattaagcagcagatccgggctatgaacccagagatcgtaggcctaaagtctgtgggcttaagcactgtgctacagcgcctcgcttgagggaaaggcagaggtgctgggaggatatgaggaaatgagatagggaggaaatgagatcaaagatggcagaagaaaggatcatagccagcgtgagaaaaagtgcagccgggaaagagctagataaatcatataggcagagagaggggtaggggagcctggcagcaacaagctggggtagacagattgaggggagccataagggcggcaggcacatggccgggtgggggatcaggacgggagatcccggagaggaagggccatacggggaggcagaagtggacgggcccacttgggttccagggtccatcctgcgtggctttctgctctgcccactgagtgggtgccaagggggctatgtcctcccactctgcagggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggtaagtgtacttgctttagggcaggctcaggggttcgtggtctcatttctttggggctgtgagctgtccaaggtcaacgctctgcaggaagggaggatgtaatggttgggtggggggtcccccctttggaggtgggaagtgaaccctgtaccctgaggatgtggcttggttcccctggcccaggatggacacataggggtggaatgacagggcccagaatcacaggtccttggcgtgggctagcaccgtttccctagcttcatctgttgggcttcccatcccagcatcccctgggagcggtcctctctccagcctggcttgagcagcccagggtggggaacatgaccacctgggagcagctcctcccctcagtaggaagctgtgtcctatggactcagctgtgtgtagtttctttcatctgctccttgttcatggactggccaagtttcctgggggtctgggctagctgaggagaggctttgagggaatgcccacctgggctggaccatccccttgacaacaaaggctcggagccccttcctctgtcaggatgccctctttggcttaattttaatctttccctcagcatcatgacaaaagtgctcacctcttggtttcttacactctaaaatgacagtgtcagtaaaactggccctacctgctcatcggatgccacccctgacggcagggcccctggcattgtgtcgcccacttaggcgcaggccccacgcactggctgtgtgacctccagagagtgacatgacgcctttgtgtccacacttcctcatgtctaaaatggggacgctggcacccagcggccggtgtgagagacagggatgatttatggaatccgccctgaagtgtcacttcagatatggcggtttatgatgactggattctagaatgatttttgagactcgaggtcaaaatattgtgaatggaagtggggatggggggttgcccgttcccctccttcccttacccagcggctgggggtccatttcccaggccttgcccgctccggggctgctcgcgatccccacgaccacgtgcctctcctctcccgcaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggtgagggcgccgcgggacccgttccgccccagcccacgcttcttctgctgccaggcttgctgcattctgggccctcggcttctgaccttttcactacgtcctgggtgcctgggagatggaaatcccattaagcacttatttaattccattaagtggcttgttgcgctattaaatgtccattcatcccattacattcttccactcccagagccctggtgggcccctggagctggctgtcccgggacggcctggagcagggatgccctgggcaggagacgtgcccagcctccgaacaccacctggccccctgcctgccagcgtcctggagcagaccgtttcctcttcctcttcctggtctcagggctgtctcctggctgcttccttagagtgggacaggggtctgacctctcttgtttcttgtgactcccctggtctcttgagctgaccccatggactcctgactgtgcagggcctggtctccactctcgatctatgggtggcctgcctggcctggggccctgggctgctggcctgaccacgctgcctctccacctaggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtaggcgcccagcggccgggctctcctctcaccttggaggagcttggagagggggtgggagtgggctgtgatccagagccgggtggagggggctagccagtgagggcctcttggagggaggcagacctgagggttggggttggcaatctattccagagctgcgatggggggagaggggacagtctgagaagtcctggagaggggactggagaggtctggtaaagggctgggaatggcagagcggaggtcgggtggatggggtgcgtgccttcggggcacagggtgaggacagggcagttgaggctggggacatgacagggccagtagcaaggggggaaggcggcctgagggacatgaaaggtctggctgcagggtgtgtgaaggaagtactaggggccaagccaactgggaggcttcaaatgagaaggggtcacagaggtcaaggacaggagggtcctaattcttccaggccaaggtgatgtgccaggcagagggtgggcctcagtcctgtggggatggccaggatggcctcgaggggcccaactatgtccctaaagcatgggagaagcaagttttcttctcatagggaggtggtgggtacaaagatatggaagggtcaggtccctgaatcctgggccagagaagacccctcagccttgggggcattagccagcctggtcttctgtcccctcctgggtttggggtcctcctggcagcaagaggacctgcgacccctccatggcttcatccacccgacaccttatcctagcctttgggggctgggggtgggtgggtcaggagtgcagatctctgcagtgagtgagaagcctgggataatgtggaggactcctgtcccaagctgttccctcctgccccttccacagcagctgcctgggttggctcatgtaagggagctgccctgctacctcggcgggctcctggccctgctcccaccgcctggctttctgggtccctgccaggtctccagcccactggccttgactccagagcattccccctcaccccaggcccttatctggccacctacccctctcttcctcctcccttgcttctctcttggttcccacctgagcccctggcatctgccctgctcctgtgcccgctgcatgtgcccgtgagttgcggtgggtctgtgcatgtgcgcctgtatgtgtcggcaggcgtgtgtgttcctgccagtcactagcagtctctccatctctgggtgtctctgtctgactctggctggctcagcactggaccggctgggcgggggtgtcagggagaccattaatctgcggtgacaggcccggctgctggcggagggggaggggctcaggctgcgggagcgccgctgcaggagcctgggcgggctgcgggggagggggccgctggctgggcaccggcggggcgggggagcctagtgtggcggggtgggggtggggacctggtagaaggaggggccggctggaggagctgaggttccgagtgcggccgctgctgggctggcgggcgggcagagcacggcaccctggcagcagggcccacgccacggggccatgggcagctcgagccaggcaggctgctgcccacgcttactgccagggtgaccccagccctggggcccagccacaaccaccctggcttcatgccaggggctgctctggttgccagtcggccagcctcgggggtgcagcctgggctgggactgctgctggggtgcaggtgaggcagtggccgggccctcaggccccagggcaggcaggctgcagggagccaagtcctccatggcggccccagccgggaaggcgagcaggacaggggctctgcggcccagggcccagaaaggccgggtgaggcgggccgtgcgcatctccagcctcgtggcccaggaggtaggtgacacccggtccttcctcttctttctggggccggcacactgaggcaggtggctggcccaagggctctgactccaggctggggagagggctgctgggaagtaggaagctttgggagccgtggggcctggccctgctcacccgcgcctggctcctgttctcattccctcccaggctgggcttacagagtttccgttgctgggacttccccgctcctgtgcctgccatggcccacagtcctggagctcaaaatgcaaagtggggagatgacaggaatgagatgggtgcccagagagatggtggggaggcggagtgggacaaaggagcccatgggcaggaccaggagaagccggcaggcgacagctgggcctggcatggaggcgccggggctcctttgctctgtgtgctggggttggaccctgtgggcaaaggaggcttgtgctgttttggggggtcgaggaggctggaatgtgcggaagccactgtgtgggtggtgagggcgcgctgggctgaggcacctggctcatctcttcatgggcagccgaggtgtggtggctgggccatgcactactctgtgacctcctgggagccatccagccgctcttctgtcagcctggggcaggcttgggccgacagtggaggaggagtgggaggtggctcagggctaagtctgggaatcccatccctgcccccaccaccccacatctgcacattctgccttggctcatacactctttccagctgggggacaggtacaggtaggtcttcccacccacagctgaggagtcagggtttggcttaagcaagccagcttcagtattctataggacaacaggctggggggacagacctggcccaaggccataggtgagctggtggctgcagagctgggactggcccctgtcaggtgtgccccatgcacgtgtgagtttgtaccctggggcagtgttgctcatgctgtgtgcagactcaggggtagggtcactgctgttaggggtggtcctgacacctccgcccagaggcttgacctctcaggcacagagaagagccctgtccaggggcagctaccttcttctgcccccttatcagcaggggcctggccagtgttttcttttttttttttcagtccccacttgatcactgaagggccacccacccatgggcatttgctctctgccttaaggagctgggcacttagacagcagacagagcagaaatggagcctgtgagcctgtccatgtgttccctccctgccctaggcctcagctgctcccgcagcccatctggtaccgctggaggccctgtgtccaccccagggtggtcaaatctttgactatgacccgtcttgacttctagttatttcctctaaggagatgttctcccagcagcgccatctgtgaaggtgtcttgggcaagcccagcttgagtatgaaggggagtctttgggactcatcaactatggcctccccaacccaaaagcctgagggtgtgggggcctgggagtcagtgcttctgggagcagggccagcacctctggaattcatgggggctgggcaaggccttgggttcagctggctgcccagggatggcagggagagcttggggagggggcttttgggttgagctgcatgcctgagagaggattctagggagacccagaagcttctgggacagtgtggagaagctggccggagctcttcttagtttgccttgggagcaagagggacgtgcctgctgtcaccgggtccggatggccagcctgcctctgtctgatgactgtggaggaggcagagtgtgagtgtgagctggtacaggggctgtgtgtgtcactgctgagcagtccctgtgtgcacgtccctctgtataagttgggtgccatctcctcccagctgggctatgtcttctttgtccctttgtaagagggtgggaggatgaggactgtgggctgtcccggacctgtgtggagagggggctgagggagcgaggtgtgcgagtggagggtgggagtgtcttggtctttctccatcctggttgcctggtgtcctcgggcctgtggcatgaagaggggctggtaagaactgcagtgactaccagtcagtggattggggctatggctcctggggccactgcccaggttgggagctgttgtccttggaaccctttccggtctgagactatggcgtgtgtggttgggtgtgtgggccagcgtctcagggcagtcacagtggcaggtctgcatctgcattgccggcggggagcagcactgggtgttcctgccgagtggcctgaatccacgtctgccgggaaatgttggaactgtttggagccagtcctgtgctgtgtggcattttgggaaccatgatgcccaaacctgggggtggccatttgctgtggtcgcttggctgaggggtgggcattctgatggaagctttttttggcctcctcctcattctgcttggccccggcagagatgtcatcgctcctgcccccgaggtcccatggcctgcctcacccacacctccgccttccccgggtgcaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggaggtggggtcagcaaggaggcaggtggtgtagggagaggagaggccaggaatgtggtgggaggtagtggggtggagggggtggcgaggaggacagagaaaaagagagagagagagaaagagagaaagacagtgggagagagacatggtggcaaggcagctgcagaggcaaggtgggtaccccagcaaacctatgctccttctctccacacaagctcctcgcaggttgcagagggccacacaggacacctttttttctaattcgtccatcccaaggggccattattttgtagcgtcaccaaagtgccctagaagctggtgggagtgggcaggcaccctagggtgatcactgcacctgtcagtgcccctgtttcctcatccacactatgggggctgcctctgtcccaaagctagcactaagattaatgagacacctgtgctgggcccagtgtgggcttcacctcttaggaggagggtctaggaagtctttggggatctgacctctgatgctcgctctgagggccgcagcacctcccctcctcccttgccccccttgccccatcaacggaatgtgccccttccctgtcccccagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctgtgagtgtgcccaggggcgggcggcggggagagcccacggtggaggaacccaagttggaggaaactgaggctgctagccgggccagagtgaggccctggaacttagagactccaggggccaccatcgtgacatggtttgggggctgggtcaccagggcggggactactgaagggtggggtgtgctggagcgcagatgtacaagggcgtctcgtgggcatgtcgacgctgagactgagacactgacctggtgcggggcctgagcagggtccctgtgggtgggtggggtccctgcagaggctgacggccccacgtgcccacgcccccagccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgaggccaccagagcgtggccagtgggtggcaggctgggaagagtggggtggcagaggggaacctcttggcacagcaaacaagtctcaggcggtcacagtcagtagtaaggaccctggaggcacgttgcccctttttgcccatgaggaaatggaagcccttggaaccccttccctggagagctccccttttggggtgggaggtatatgtcacataggcgatggggttaaatccttgtgacagcccagggcgggaatggtctctatgccttacaaataagaacatggaggtctagagcccaagggtgctcggccaggcatggtgaagctggagtggggtcccaggtccttcccaagacactgggccccctgatgcttccgagatctcccaggcctggaggttgagatttctctgacatggaggggtgggatggtggagtagagtgtgggttggggggtcccaagggagggtgtgctgagctgcccccaccctgcccccaggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctgggtatggggtggggggcgggcactggactggaaatgccctctgcagcctcaagaggtgcgagccttctgaatatgcagtcactggggctgtggacctgggactgcctgcagggtcactgggctcctttaattcacctaaactcaggccctccaagcggggccatggagaggagccccacgtggggtgaggctgctgaactctggggttcccacattctccttcccttcaggatccgccacaaacagacactttttgcttccttaaagtaggatcaaatctagatcctctagcctgggcagtagaggaagaaatgctagcctggaagctcggcatttggtttcactaagggccatgtggttccctgcagcctcatgcctggccccttgacacatccaaagcaaagggagtcctgccccctccccccacttcctttctaccctgcctgtgcacagtgggtgggttggtgtgtctggacactgaggacttcctccccctttgcctgtccttccctcggccctgtgtgcctcagggcagatatagcaagctctttcgaccatagttgatggtaggacattttagactttgtttctcagctctgtacaaacacaaatacacacccccacaaaactaaaatcaaagtttcactacataacactgggccttactgcatgtggttcattctagcatttctgttctgtgctgtgctaagctatactactgtatgttctttcagtaaaaaaaaaaaaaaaaaaaaaaaaatgctggttttgattcactactgtgttctgatctttggtttgaagaacattgcttataagggtgcagtgattggctaagagggtgtttgggacctggggtttggggtagtcctgctggctgggtctcatttgctgggagaccctgggctcctggcccctctggcactgtcgtgcctgctgggagctgagggagggtgggctggcagattgcttccccggttgtgtgggttgggggccggctggcaggcgacccagctgaggggacatgctctgcccggggtggggatgccgggagaaggtgcctgctgcctggatgccgccccccggggctgagctccctgtcctctccatggcctccagggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagatgtgagttggctgccctgagctggaccccatcctcctgcttccagctgtgtggctgcccctttgctgtgtgacattgaatgtgatgcttctctgtctcagtttccctgtctgtcaaatggtatcatagagcagcctacaaaagtcccaaactcagtctttgcctggggcaaaatcacattgggggcagggaagggttttcccagagctggggcgcccagccctactttttctgtgttaaggagggagcttggggcctgaccccggtggggcaggagagcactgaaagggccctgatactgattttggttccagaccaacatgatcccgggctcccccggcagtacggagttagagggtggcttcagtcggcaacgcaagcgcaagttgtccttccgcaggcgcacggacaagggtgaggcgggggaggggaggaagggggagggcggggacaaggtgaggctgggagctggaaggcggggtgggccgtgtctgctttacccaggcctcagaccttcccatcccttcctccagtgtgcccactccacaccccagctgtccagatgctgcccatctttcagggcccctgcagctgtcacctcctccaggaagccctcttccaccctccagggcaagcctaggctttttggacttcacagggctggggtctctgtgagcactgaggaggaaagaacatagaacaaaggagggccagggagccaacgatggggctgaggccccgcagtcccagagatccgccaacccctgaagcaccattgccagtgcttgggcagtcagggggtcctgggagttcttggtcggcttgacagtgggtgcaagatagcagaagaagcgactggggacaaaccagggcttcctgcccagtcctctctcgggctcgctcctgtcccctcctccaccctcgccccctcctctctgttctcctcccctctctgaggcccattctctgtttcccacagacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggtatcccgggcgacgggcgggcgagggaggaccgggcgtggcagcggtggtgcgtctaccccgctcacccagctctgctctctggctgcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggtgagggagtgcaggtggggtgggggggcacgccctggagtctggtccaggtcctggcggtgttgtctggtagagggagagggcctgtctgtgtgtgcagcagctggatccccttcttccattcctagcccagctgggggctgccacacctctagcgcggagggcctgggttgacaggctgcccctcccccctccccttccccttccccactcccggtggaggctgtcactggtgtccccacttctctgagcatcccccacttcctgccccaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtaagctccagccctgcttcctcgatgtaccgtgacccgcttgttccccgattccagcccaaacctgctttgctgcctttctgctgggctctgtggcccagcctccctgaaccttgttcctgccctcctcccacccctgtcctcctggaggaaggctgcttcaggagctgcaggctcagggtagggcaggagagtcccccgtccaaagcccctagacagctgggtgcagggaacctgtaggctaagtggcccgcagcttcactcccctagtgtccacagcctccaccgccaggccccggagcatcgcctgaagagccagctccaacggtggtgcctgtctcacggccacgggtggtctccaagaactgactgagcccgagacgtggcggtggaggctgcataccagagtatctagggacagggtcccgggtcccttctgtgagtgacactccctggtctcaccgtcctgcccatccttgcccttggcagtggctgtcacagggtcatcttgagggtgcttggcatttcccagggtgcagaggccaagcctccaggggaggacttcctgggcctgctttgccccctctgctagagatgggccttagttgccccttgtcctctcctgggtggacctgcatgttcctggcctctgcaggatggcggtgtctgtgtcaacctccgcgtccatggtggggactcctctggctgccagaggcggcctgggatctgctccctccccacttcccttggcatccgctcccccctcccatatgctgcctgctcctgtttgcagacacgagggcccacgtcagcaaatgtgccagtctcccttcgcagccgcccccgcagagctctcaccagcagacgctcagtacccacaaacccctcccgcctgccccggccctctccctggccttcagatgcctggccgccagcctgcctctgtttctaggtccccgagttctttctggattgcgggaaggaatcttgtctctttttctgtcccagcttctgtcccaacacagagactgcttgttccaggttgggttgggctcagcggccacattccctcgggaagagacaggggcctagggttttccctgctcccagctctcttgtttggctcctgctctcagagaatgcctttctctacttcccaccttggtgcctccccttggtggcccctgtcctgctctcgctgccttggctcttgtcccctccagcttctctgcccacctgactctcctcttggccctcctgtcctcccgtccatcctctgtccccgggttctcctgcccctttccctccccttcctcctcctccatggcctcttcgcctgcccatgctctgtgtgtattgcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttagtggggctgcccagtgtggacacgtggctcacccagggatcaaggcgctgctgggccgctccccttggaggccctgctcaggaggccctgaccgtggaaggggagaggaactcgaaagcacagctcctcccccagcccttgggaccatcttctcctgcagtcccctgggccccagtgagaggggcaggggcagggccggcagtaggtggggcctgtggtccccccactgccctgagggcattagctggtctaactgcccggaggcacccggccctgggccttaggcacctcaaggacttttctgctatttactgctcttattgttaaggataataattaaggatcatatgaataattaatgaagatgctgatgactatgaataataaataattatcctgaggagactcca

SEQ ID NO: 10 - protein, 325 aa, Homo sapiens
QPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 11 - RNA, 3083 nt, Homo sapiens
  source 1..3083: mol_type = mRNA
aaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacata

SEQ ID NO: 12 - DNA, 14082 bp, Homo sapiens
  source 1..14082: mol_type = other DNA
aaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtaagtttttaaaaaatgagaacacgttacctattagtttctttttctacaagtaaaacttttctaaagttttaaaatgtgttaaatgtaattttgaattatgatactgtaaatggattgatggatttgaaagtgtttctatagttttaaagttaaaatttattgtaccaaatatatattttgactgttaaaaaagtttctattaacattgcaacaggtgaaaaggttcaaacctttccgattagaataaaggattagtttactggatgtttggtttttcattttagagtatcattttattttgttttttttttgtaatcagtaatagtctaggtgctatcatttgttgggtggggggggggcggttaggcgcctgggttgggggatttgactgactacatcggaaaaatcaaagtgcaaatttgcttagcgtgcttcctatagtattctacacttcatagaataaagtgcttttttcatatggaaatcttgattgatgttctatagctttgcaaagccaacttcttttgtgacgaagtgctttgttaaaacatcgtggttacttgacttccacgtggttccttcctagaattttccccattaaacaaaaaggtataaaattcaagagtattaatattatgctttaaaaatcaacagtaaattgttactttgtgttagcaaccgctatggaaagatttgagaccctcaggagagacagcttttactcgaccacgttggcagtttaggagatgaaatctcttgaatgggtggtggctagttacagagcattagagatcagacgtttaaaaaagagtgtatgccagggtacttttccaaaaccgtctcctaaaacgggagctcctagaaagagcaggaaaaaaactttcttaaacagcgctttgggagctccccccgccaacacccgccgccccgcaccttccctctgctcgaacctgtaacagaattcagaactctggggcagttctttggagttggagctcctagggcaaagataacgctaatgcgttgtttaaaagggttcaatttttaaaatacttttattggtaacacccattctgttcatcagaaatgcagccatgcatttttgtgcccggtaaagattttctgttctgtgtaaatctacaaactaaaataattagtttagattcttgacgtgaccctgcagtggaagtatttgcaccagtgaatgctgaaaagcaaataaaaatgaaagctttgcggaggccgtgggccggacgggcgcggggcgcctcctgccatcttagcgtctgccgggatccttcccgcgccttcgctgcccgaaccagctcggtcttgggacgtttcccccaaatcctcacaagtcaaatcgaactcaggtccgagttcgtcactgcaaaggaacccggggtgcggggtgggagggataggaggagagatgggctgggtgccaaatggtagaacaaatggttgatttaattaaagttgtcaatctaatttcccatccctctcacaccagccctttttttttttctttttcttttttcctcttaaacttgctgaatcccactatctccgcgttggtaagaaaacctgaataccttgggaattgtgcttaaattgcacttgcaactcccctggaagttcctgaaacttcccgagaatgaggcggggcggctcaaggggaaagggcagggaggagagaccctcgggaagctgatgacgcgtccggggcaagtcagcctgtttccgagacgcgcgcctcacggtcgtagcagcgctgcgcccaccctgcccctttagtgacccagccagcctttgctgggggtttctgataacgctcatcgataagctctccttgctcccaaaaggcgtgcttctctttctctccaggtacaaagtaggggtgggaaggggagagctacacaatgttgtaaaaagaagcgtggctttatttggaagacggttgaatttcttttgcttcctcgacccagttagtaacgacaagaaagatactgaatatcgccctccgaaaatcgggcgcatttgaactcggtgaaaagtgtccctgtgaggaagcctaatgtttacttttgtgcaaggataactggaaattaaagcgtctagtaatgcagagatttcattaagcgagcgctcccctcccccccgcgccaggaatgaaataagccagttagacagaaaacacttttgggagaagactaattacttctgggtaggccgaattgactggaaattataccaagcaaaggcagtacattccattccgtcctggcgatgttgattctatattattagaacattgttcccccagtgtgggggtgctttgtacttcccaaagtcagggtagactggttacctgggaaaatgcagtgttagtgctttgctcctactttcttcactgtttaaacatgttccacatttttctaatgccttctgagggttctcaacccagccactgagttcctcacccttggcgactgtgataccaacaatgctgtcaccctccttgggaaacatggttgcaaagatgctccgcaggaggcagataaaaggctgtgctttcagatactgaaggttgctttgttacttagatgtttccaattcctgcatagagaaggagggaaaacacggggtgcattctttcttttcttttctctacatccaggggtgtagcttggtttgagcctttatatatgatacccgcagggaagggaatgataggacagtcaccagtgattaataccttccactccgcctttcctgttgttctgacttatcttcaggattctcagttatttgaagagatgtgtgggagcttatccgcacacaccagttcaactctggagagaaaaagtaattatctagtgtttattattaatgtaatattctgtatggataattcaagttttaaaaaatctccagagcatctcttccatgggagattaatgtatatattttttaaattcttagcataattgtctaaacctccatctaaagtcaccttttaactatgtatgtacagattgtcttttacctattttcaaactgtggttagctctttttttttaatacatcagtggcttatacaatttcataaatttcacattccaatttccctatgcttttaaaataatattttaagaaaaatatatgtttatcgtcagctgggcatgatggctcatgcctgtaatcctagccctttgagaggctgaggtgggaggatcgcttggggccaggagtttaaaaccagcctgagcaacatagcaagaccccacctctataaaaaaaaattttaaaaagaaaatgtatgtttgtcaatcatttcttagttgctgttttgctaaagaaaagatgaaaaatttaaaaccaaatgagctatatctaaaggtcgtctaaagagtaagaaccaaactttactgagctgttactaagagcatcacatgcatgagttatttattaaattctcattgaaaaaaatagactatcttaatcaatacacttgtgagggccctaagggtgtctttatatgattataaaataacaaggctattcactaaaaatagtcaatttggaggtggaagtaagagtaggggcagaggaaacgccaaggactgatagaaatatattaagttacatcacaattgaagaacatggcttttagcctcagcttctgacttgggattttctctctggtagccctaaattgatcttgagaaacttgttttgggggagaattgagtagtagctgttgaactagggagaggattgtcttttatgggatgcaccatcctctctttggatggagttcctcacacctcaattcccatctcatctcattaagtggatgctaatgtaagttcagtgatgaggaggggtaagagctggggaaggaaggcttgttggtgtttttatgttgtcctcccctctgcccccaaaataagcagttcacaacatcaaatacctacacaaatgtgcaattatttataaatatatttttacatacagtaagtttgatacactatagttaggccatctaggtataaataaggaagtaaaggttgggtattatctgatgaactgggaatcttttccatggtacttgaatctaatcactttagtgcctgcatagactcagactgtaaagatgttacattaaccagggttaatatcaactgctgctcttccccttcagagccctagccttccccgatatttatcattcttgaaataaacacgattttaaacaggctatttcttcttgtaatagtgtccttgtctacaataaaattgtgtggtgcaatcggagttagaacatcgtagaactaggggacaaccacacagttgtgttttttcctttttttttttgttttgtgttttaaatcttaacctgcaacaacctaacagtgaactgaatgtacaagcatgatacctgatgcagttctacaagaatcctaaccctcattaggatctcatttaacaaattcaggaagctctttgttaaaaagccttttttcttttttcttttaagcctgtcaaatttagcagtgggggttgcaaagagccttttttcttaatttagctcttttgcctgctcaggaatcatttgaagcatgttgtttcaacaaacagttctctgtttcagcaattgtcacttgaaaatttaacagtatgagaatgagatgatgacaatagttttttactgtgaagtttcctttttaattaatttgaaactttagaaatttgagatataactacaattaagagaatcccaaagagacagaaaaaaacaaactttttttttttttttgcacagtatctttctattttttgtggaagccccatttaattcatttaacatgtagctataccccaattaattaattcttttcttttcttttctttttttgagacggagtttcactctatcacccgggttggagtgcagtggtgtgatctctgctcactgcagcctccgcctcccgggttcaagcaattctcctgcctcagcctccagagtagctgggattacaggcacccgccaccacgcctggctacttttttgttgttgttgttgttgttgttgttgttgtttgtcgtttttttctttgagacggagtctcgctctgtcgcccaggctggagtgcagtggcgcgatcccggctcactgcaagctccgcctcccgggttcacgccattctcctgcctcagcctcccgagtagctgggactacaggtgcccgccaccacgcccggctaatttttttgtattttttttatttttagtagagacggggtttcactgtgttagccaggatggtctctatctcctaacctcgtgatccgcccgcctcggcctcccaaagtgctgggactacaggcttaagccaccgcacccggtctacttttttgtgtttttagtagagatgggatttcactatgttggccaggctgatctcaaactcttgacctcagatgatcctcccacctcggcctcccaaagtgctggaattacaggtgtgagccacatctcttactctttcaagcactggggatacagcagtgaacaaagcaaactccctcgtattgttcacattccagttaaggaagacaaataataaatccttctcatggaaggaggtacatagtaatcaggcaaatatttactatatcaagtagagagaaagtctattaaaataataattttttaaaaaccagggcaaagggaataggaaatgacaggactggggaaaggatgttgctatttcatatttggtggtcaggaaagttggctgatgaggtgagacctgcaacaagtgaggacgtgagctgcgtggatggagcagagctcatcaaaccaagctgattgttggtttgttttctcaagattgactgattactcggagtataggttggcaagtagggtgaccaggctaatgacatgaaacctagcaaactgatgatggctgagatcaagctaagggtggctgttttaagtgcggaggaatagacaactgagttcactcaatgacatttactggaggggcagaggttattataaaagtgaaagaaaacattacaagggtttgcatttgagtttttgggcatgaatatttttgggtgttagtacattagacacacacatgatttccctttcctcccaaaaaatccattaaatgattttatcgggaacttccatagacatttccttatgtaggtttagactctgattttattctttcagatattaaagtccttcaccttctcttttctttcaatcataaaaagatgctggatatcaaatttcactttagttttcaataatcctatttacctttatttaagtctccattctcaactcattacatttgtctcttcttgagaaaaatatttaaagtcgatagtaaaatacttaattctaataactattagtatattaatggcatggaaagacttgactgcaacttcggcacatgctctagcaaaaggcatagtcaaatgtaatttaaaatcaaagctttatttttaagagatctcatttgaattaaagcgtttaagttcttctgctctcagggagctgcagatggagatgtaaggagtgacaaggggctgtggagccaaggctgacctcaatgccgggtgcccagagagctgctgctcctggtactcagccccttggattttttagactgttccttctgctttcctgacatttctcccactcctaaatattttgaacaagaaagggccagaaatgtcaagaatagtaacaaacaccaaaaaaaccttacttcctaggccccacctccagaaattcagattcattcagactggtgaagaccaggaatgtatgtttttaacaaatgctgccaagtgactgtgatgcagttcaggaaaggggccagaggagcctcaggctggtcacacttagtgcctgtagctttgggcatccatccagtctagggggccatgaagagcaatggaaatgtaatctaacatggtgcatagacggcaaaaagcgtggtccagctgctaagactctgctcttaacaagaactttgcacaactcctcttagacagaccttggtctacatataaagcaccaactcagtatcctagaggtgaaggcatgaatatatgtgatatgtccagctgcgttggtcttctcatgtatcaaataagaaatatacctgtttccccctggtgaataaaagtgcagtttatagtctgtagatcaatgccagggattctgtaaacatataattatgcccttttattataagagggtattaacttaccatacaagacttggaaaaatgaagatattaaactagtttgggaaggtgcagcagggaggaacaaccagttgccaaaatttgattcatggcaaagtgagcaactaccaaaaaaaaagggaacaatttttgttacctgtttttgggcagctgtcacattaaacattatttgatggtatctatgatagtggagatcgacaggtagctacatgttacaggatactcggtgtccaattcgataattcgtatcttattaagatgaaagagtataactcattaatgcaaatgacaactgcatgggtgcttaaagaaaagacaactttgcaattaagttactagagtattaaaaaagaactaatgaagagctgtctgatgacttagttcacatactatggaactgtggagtgtttttgtagaatctcaaaattatatgaaagtcatgtatcatgaatttttttatagaatgtaaataaagactatactttcagggatcatttctatagtttgttactagaatgtaaataaacttagtgatgaattactataagcatctacacaatcatattttactttgagtaatgatgaaatcctagctaaaataacttctgttctgcctggtcatatttctagatatgataggtttgtcttcagctctatcgttgcatgaatgtgatgagatgtatgcaaagtttatctttttatacttgattgtttttgttatttttcccctagcaattgaaatatgtgagcaaaagcagatgacactggcaaaatactgtaattttttacaaacagaattagggccaattttacacatcatcacatagaaactgtctattgatatagctccaaagattctgtttgtctgaccctcatggactatacttcttcatttccagtctagtattttctgctaaattgtagggtttttttttctttcacattctctcaccatcaaactgtccatcacggctaaattactccctcacttcaagcctttccttggctattcaactgtctttgtcagcctttcaagcctttgttcttatttgactgctgttttaaattgatgctgttttcatacatcaatttcacttcctaacttgtcacttcagaatctaaaatgacccttcctaagtaaaagggaagctccctttcttatgaccactgggggagtgttatttctgtggccttcacacccacctcccactcttactcagcattctttggcactagtccctgggctgtcatcaaatgtacccatgataccgtggtaccatctagctggtgcatagtttagctttatggctcagccatctcttctttgttcggtccaggaaatcccaggtcaggtgcccatttcattttttcttgttctctatggccagaagatgcagtcattagacagaaatgtaatgttgcttactgaggaagaagactcccctggatttcatttacttcctacaaggagatacagctcaatagttcaacaccagaagagaaactcctttatattcagatagtgaacatatttctaagaagcctaaataatcctacctttagatactctatttagaattcattactaagctcctgcaaagtcttagctttgcttgtttttaattattaaacaatctttgactaatttgtccttttctaaatcctccaattactactatgtgctgaaaattagcctatattccctatgcctgccacatcatgttactcacttcttcccagctttcagccctggctaaggttgtgtatatcaaagcctggaaacatttctagctttagcgcagccttactgtacttttaaaaccagtgtgaggcattgaaaagacttgcccggggattgtgaatcttcttctggctctgtctatagctaaatatttcctataccctgagaacctctgagaacctcaatttttatcagctaaatggataatcggcaattagaagcataaattgaactagacaactttggggattcttgttggtttctactgacttacatctttttagtgagacggagtctctctttgtcacccaggctggagtgcagtggcgtgatctctgcaagctccgcctcccgggttcaagcgattctcttgccttagcctcccgagtagctgggactacaggctcccaccaccacacctggctaatttcttgtattttttgtagagacggggtttcaccatgttagccaggatggtctgaatctcctgacctcatgatccacccgcctcccaaagtgcttataggcgtgagtgcattataggcgtgagccaccacgcctggccagatctttttcttatcctcagtaatggagaaggcgtggactttagatagctagatattgataagaagttaaatgggatgtatagcctgagggtccaaagcccagtaagggaactttaaagtatataacaatgaaagatgaaaaccaatcttttctggctggagaaaacaccctgaagtaggttttgtttgtttgttttttaataaggctgtgagtagtattgagacaaaatgaactttttacatgaagtgtgatgtgtatttagtctttgtttttcaaagtaggttactacatataatgacacattgtagcaaaagagtatatttccatacagcctactttttctctggacagaaggctgacttctgagctacataagtcactgcttaagaaaaaaatagtcaccattgcttgggaggctaaggcaggaggatttcttgagcccaggggcctgggcaacatagtgaaatcctatctctaaaaaaagaaacagaaaagtgtaaatgacaataatatagggtatgcacaaattacacttgatataccattgaaaaatcttccagaggccaggcgtggtggctcacacctgtaatcccagcactttgggaggctgaggtagatggatcacctgaggtcaggagttcgagaccagcctgagcaacatggagaaacctcatctctactaaaaatacaaaattagccaggcgtgatggcacatgcctgtaatcccagctacttaggaggctgaggcaggagaatcgctggaacctaagaggcggaagttgtggtgagctgagattgtgccattgcactccagcctgggcaacaagagggaaacaccatcttaaaaaaaaaatctttcagagtgaaattagtaataaaatgatatataatagcattaaggttttggaaagaaggttatggatgtgtgtttataggtattcttacctggattcttgatacctatgctttgaataacacacatcaggattttgcttagcaaacacagatctaccaggcaaaaactgtcctcatttatcagacaaataaaaggagcagaggcttagcacaggtggcttctcaagtcacacagcaaggcagtggttgagcaagcaatacaccttcccaggttaaagactgcaactcatcatctgttgcttctaagacttactttgtctcaacaaacaaaacaaaacacttgttagatcttttcttcgttggcaaaaatgggacaggaagagtttgcacttggttttttgtgaaagaatgttagaaatacgtgaaaccgttggtagtattttgactatcacctgaaaggtactaaatgttaaactagtaatttgcaatctgtgatccttgaattgtctctttgtttctttcaggtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacata

SEQ ID NO: 13 - DNA, 4104 bp, synthetic construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 14 - DNA, 4272 bp, synthetic construct
  source 1..4272: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagtga

SEQ ID NO: 15 - DNA, 3306 bp, synthetic construct
  source 1..3306: mol_type = other DNA
atggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggctaa

SEQ ID NO: 16 - DNA, 3156 bp, synthetic construct
  source 1..3156: mol_type = other DNA
aagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 17 - DNA, 3147 bp, synthetic construct
  source 1..3147: mol_type = other DNA
atggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtgtaa

SEQ ID NO: 18 - DNA, 2949 bp, synthetic construct
  source 1..2949: mol_type = other DNA
gcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaa

SEQ ID NO: 19 - DNA, 4107 bp, synthetic construct
  source 1..4107: mol_type = other DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgactga

SEQ ID NO: 20 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - protein, 1118 aa, synthetic construct
MDYKDHDGDYKDHDIDYKDDDDKGGSPKKKRKVAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGS

SEQ ID NO: 22 - protein, 1145 aa, synthetic construct
MDYKDHDGDYKDHDIDYKDDDDKGGSPKKKRKVGSGPKKKRKVGSGPKKKRKVEAGSARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSPKKKRKVEALASGGGSGGGSGGSGSGGTSRGFARGLEPERIIGATDSSGELMFLMKWKNSDEADLVPAKEANVKCPQVVISFYEERLTWHSYASGGGSPKKKRKV

SEQ ID NO: 23 - DNA, 288 bp, synthetic construct
  source 1..288: mol_type = other DNA
atgggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctc

SEQ ID NO: 24 - protein, 96 aa, synthetic construct
MGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 25 - protein, 57 aa, synthetic construct
VTFEDVTVNFTQGEWQRLNPEQRNLYRDVMLENYSNLVSVGQGETTKPDVILRLEQG

SEQ ID NO: 26 - DNA, 1212 bp, synthetic construct
  source 1..1212: mol_type = other DNA
agcagggctgaccccaagaagaagaggaaggtgagtggtggaggaagtggcgggtcagggtcgatggacgcgaaatcacttacggcatggtcgagaacactggttacgttcaaggacgtgtttgtggactttacacgtgaggagtggaaattgctggatactgcgcaacaaattgtgtatcgaaatgtcatgcttgagaattacaagaacctcgtcagtctcggataccagttgacgaaaccggatgtgatccttaggctcgaaaagggggaagaaccttggctggtatcgggaggtggttcgggtggctctggatcaagcccaaagaagaaacggaaggtggaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcatag

SEQ ID NO: 27 - protein, 178 aa, synthetic construct
MRTLVTFKDVFVDFTREEWKLLDTAQQIVYRNVMLENYKNLVSLGYQLTKPDVILRLEKGEEPSGGGSGGGSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRD

SEQ ID NO: 28 - protein, 403 aa, synthetic construct
SRADPKKKRKVSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGSSPKKKRKVEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 29 - DNA, 5316 bp, synthetic construct
  source 1..5316: mol_type = other DNA
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgagtggtggaggaagtggcgggtcagggtcgatggacgcgaaatcacttacggcatggtcgagaacactggttacgttcaaggacgtgtttgtggactttacacgtgaggagtggaaattgctggatactgcgcaacaaattgtgtatcgaaatgtcatgcttgagaattacaagaacctcgtcagtctcggataccagttgacgaaaccggatgtgatccttaggctcgaaaagggggaagaaccttggctggtatcgggaggtggttcgggtggctctggatcaagcccaaagaagaaacggaaggtggaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcatag

SEQ ID NO: 30 - DNA, 705 bp, synthetic construct
  source 1..705: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacattcccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtac

SEQ ID NO: 31 - DNA, 48 bp, synthetic construct
  source 1..48: mol_type = other DNA
aaaaggccggcggccacgaaaaaggccggccaggcaaaaaagaaaaag

SEQ ID NO: 32 - DNA, 21 bp, synthetic construct
  source 1..21: mol_type = other DNA
ccaaagaaaaagaggaaagtc

SEQ ID NO: 33 - DNA, 21 bp, synthetic construct
  source 1..21: mol_type = other DNA
ccaaagaagaaacggaaggtg

SEQ ID NO: 34 - DNA, 21 bp, synthetic construct
  source 1..21: mol_type = other DNA
cccaagaagaagagaaaggtg

SEQ ID NO: 35 - DNA, 21 bp, synthetic construct
  source 1..21: mol_type = other DNA
ccgaagaaaaagcgcaaggtc

SEQ ID NO: 36 - DNA, 21 bp, synthetic construct
  source 1..21: mol_type = other DNA
cctaaaaagaaacgaaaggtt

SEQ ID NO: 37 - DNA, 21 bp, synthetic construct
  source 1..21: mol_type = other DNA
ccaaagaagaagcggaaggtc

SEQ ID NO: 38 - DNA, 49 bp, synthetic construct
  source 1..49: mol_type = other DNA
aataaaacatctttgttttcattagatctgtgtgttggttttttgtgtg

SEQ ID NO: 39 - DNA, 49 bp, synthetic construct
  source 1..49: mol_type = other DNA
aataaaagatctttgttttcattagatctgtgtgttggttttttgtgtg

SEQ ID NO: 40 - DNA, 103 bp, synthetic construct
  source 1..103: mol_type = other DNA
catcatcaataatataccttattttggattgaagccaatatgataatgagggggtggagtttgtgacgtggcgcggggcgtgggaacggggcgggtgacgtag

SEQ ID NO: 41 - DNA, 234 bp, synthetic construct
  source 1..234: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 42 - DNA, 180 bp, synthetic construct
  source 1..180: mol_type = other DNA
gtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgacc

SEQ ID NO: 43 - DNA, 500 bp, synthetic construct
  source 1..500: mol_type = other DNA
ctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 44 - DNA, 408 bp, synthetic construct
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 45 - DNA, 505 bp, synthetic construct
  source 1..505: mol_type = other DNA
gggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

SEQ ID NO: 46 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 47 - DNA, 212 bp, synthetic construct
  source 1..212: mol_type = other DNA
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 48 - DNA, 241 bp, synthetic construct; identical to SEQ ID NO: 46
  source 1..241: mol_type = other DNA

SEQ ID NO: 49 - DNA, 20 bp, synthetic construct
  source 1..20: mol_type = other DNA
ggaagagtgaccttcgagga

SEQ ID NO: 50 - DNA, 20 bp, synthetic construct
  source 1..20: mol_type = other DNA
tctctgttcgggattcagcc

SEQ ID NO: 51 - DNA, 22 bp, synthetic construct
  source 1..22: mol_type = other DNA
tcaactgcgagataccaacatg

SEQ ID NO: 52 - DNA, 19 bp, synthetic construct
  source 1..19: mol_type = other DNA
ctggctgctccgtgtcctt

SEQ ID NO: 53 - DNA, 19 bp, synthetic construct
  source 1..19: mol_type = other DNA
gtgcgaaccaaccgctaca

SEQ ID NO: 54 - DNA, 19 bp, synthetic construct
  source 1..19: mol_type = other DNA
ccagcgaatgtccacacac

SEQ ID NO: 55 - DNA, 19 bp, synthetic construct
  source 1..19: mol_type = other DNA
ggtgactggagcgccttag

SEQ ID NO: 56 - DNA, 20 bp, synthetic construct
  source 1..20: mol_type = other DNA
gcgcacatgagagattggga

SEQ ID NO: 57 - DNA, 21 bp, synthetic construct
  source 1..21: mol_type = other DNA
ggagcgagatccctccaaaat

SEQ ID NO: 58 - DNA, 23 bp, synthetic construct
  source 1..23: mol_type = other DNA
ggctgttgtcatacttctcatgg

SEQ ID NO: 59 - DNA, 20 bp, synthetic construct
  source 1..20: mol_type = other DNA
ggaagagtgaccttcgagga

SEQ ID NO: 60 - DNA, 20 bp, synthetic construct
  source 1..20: mol_type = other DNA
tctctgttcgggattcagcc

SEQ ID NO: 61 - DNA, 20 bp, synthetic construct
  source 1..20: mol_type = other DNA
ggaagagtgaccttcgagga

SEQ ID NO: 62 - DNA, 20 bp, synthetic construct
  source 1..20: mol_type = other DNA
tctctgttcgggattcagcc

SEQ ID NO: 63 - DNA, 14812 bp, synthetic construct
  source 1..14812: mol_type = other DNA
tctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggccccgcgcgcagctcccggctccctcccccttcggatgtggcttgagctgtaggcgcggagtcctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatctggagctctcgagaattctcacgcgtctgcaggatatcaagcttgcggtaccgcgggcccggccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcattggtagtgggagcaacggcagcagcggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacattcccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacggcggtggcggagggatgggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctagtgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggta

SEQ ID NO: 64 - DNA, 40806 bp, synthetic construct
  source 1..40806: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcaactttgtatagaaaagttgctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggcaagtttgtacaaaaaagcaggctgccaccatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgagtggtggaggaagtggcgggtcagggtcgatggacgcgaaatcacttacggcatggtcgagaacactggttacgttcaaggacgtgtttgtggactttacacgtgaggagtggaaattgctggatactgcgcaacaaattgtgtatcgaaatgtcatgcttgagaattacaagaacctcgtcagtctcggataccagttgacgaaaccggatgtgatccttaggctcgaaaagggggaagaaccttggctggtatcgggaggtggttcgggtggctctggatcaagcccaaagaagaaacggaaggtggaagcctcagtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcatagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacaactttattatacatagttgatggccggccgcttcgaggggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagcttttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcccgggagagctcgatatcgcatgcggatttaaattaattaa

SEQ ID NO: 65 - DNA, 14812 bp, synthetic construct
  source 1..14812: mol_type = other DNA
tgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggccccgcgcgcagctcccggctccctcccccttcggatgtggcttgagctgtaggcgcggagtcctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatctggagctctcgagaattctcacgcgtctgcaggatatcaagcttgcggtaccgcgggcccggccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcattggtagtgggagcaacggcagcagcggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacattcccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacggcggtggcggagggatgggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctag